SEQUENCE LISTING (112 sequences)

SEQ ID NO: 1 - DNA, 5569 bp, Homo sapiens
  source 1..5569: mol_type = genomic DNA
agaaccccagtgtagcgagctggagagaggactgtcctgagggcagcaggcctggttgcagctggcgtgggggtctcagaatggagccctcagccctgaggaaagctggctcggagcaggaggagggctttgaggggctgcccagaagggtcactgacctggggatggtctccaatctccggcgcagcaacagcagcctcttcaagagctggaggctacagtgccccttcggcaacaatgacaagcaagaaagcctcagttcgtggattcctgaaaacatcaagaagaaagaatgcgtgtattttgtggaaagttccaaactgtctgatgctgggaaggtggtgtgtcagtgtggctacacgcatgagcagcacttggaggaggctaccaagccccacaccttccagggcacacagtgggacccaaagaaacatgtccaggagatgccaaccgatgcctttggcgacatcgtcttcacgggcctgagccagaaggtgaaaaagtacgtccgagtctcccaggacacgccctccagcgtgatctaccacctcatgacccagcactgggggctggacgtccccaatctcttgatctcggtgaccgggggggccaagaacttcaacatgaagccgcggctgaagagcattttccgcagaggcctggtcaaggtggctcagaccacaggggcctggatcatcacaggggggtcccacaccggcgtcatgaagcaggtaggcgaggcggtgcgggacttcagcctgagcagcagctacaaggaaggcgagctcatcaccatcggagtcgccacctggggcactgtccaccgccgcgagggcctgatccatcccacgggcagcttccccgccgagtacatactggatgaggatggccaagggaacctgacctgcctagacagcaaccactctcacttcatcctcgtggacgacgggacccacggccagtacggggtggagattcctctgaggaccaggctggagaagttcatatcggagcagaccaaggaaagaggaggtgtggccatcaagatccccatcgtgtgcgtggtgctggagggcggcccgggcacgttgcacaccatcgacaacgccaccaccaacggcaccccctgtgtggttgtggagggctcgggccgcgtggccgacgtcattgcccaggtggccaacctgcctgtctcggacatcactatctccctgatccagcagaaactgagcgtgttcttccaggagatgtttgagaccttcacggaaagcaggattgtcgagtggaccaaaaagatccaagatatcgtccggaggcggcagctgctgactgtcttccgggaaggcaaggatggtcagcaggacgtggatgtggccatcttgcaggccttgctgaaagcctcacggagccaagaccactttggccacgagaactgggaccaccagctgaaactggcagtggcatggaatcgcgtggacattgcccgcagtgagatcttcatggatgagtggcagtggaagccttcagatctgcaccccacgatgacagctgcactcatctccaacaagcctgagtttgtgaagctcttcctggagaacggggtgcagctgaaggagtttgtcacctgggacaccttgctctacctgtacgagaacctggacccctcctgcctgttccacagcaagctgcagaaggtgctggtggaggatcccgagcgcccggcttgcgcgcccgcggcgccccgcctgcagatgcaccacgtggcccaggtgctgcgggagctgctgggggacttcacgcagccgctttatccccggccccggcacaacgaccggctgcggctcctgctgcccgttccccacgtcaagctcaacgtgcagggagtgagcctccggtccctctacaagcgttcctcaggccatgtgaccttcaccatggaccccatccgtgaccttctcatttgggccattgtccagaaccgtcgggagctggcaggaatcatctgggctcagagccaggactgcatcgcagcggccttggcctgcagcaagatcctgaaggaactgtccaaggaggaggaggacacggacagctcggaggagatgctggcgctggcggaggagtatgagcacagagccatcggggtcttcaccgagtgctaccggaaggacgaagagagagcccagaaactgctcacccgcgtgtccgaggcctgggggaagaccacctgcctgcagctcgccctggaggccaaggacatgaagtttgtgtctcacgggggcatccaggccttcctgaccaaggtgtggtggggccagctctccgtggacaatgggctgtggcgtgtgaccctgtgcatgctggccttcccgctgctcctcaccggcctcatctccttcagggagaagaggctgcaggatgtgggcacccccgcggcccgcgcccgtgccttcttcaccgcacccgtggtggtcttccacctgaacatcctctcctacttcgccttcctctgcctgttcgcctacgtgctcatggtggacttccagcctgtgccctcctggtgcgagtgtgccatctacctctggctcttctccttggtgtgcgaggagatgcggcagctcttctatgaccctgacgagtgcgggctgatgaagaaggcagccttgtacttcagtgacttctggaataagctggacgtcggcgcaatcttgctcttcgtggcagggctgacctgcaggctcatcccggcgacgctgtaccccgggcgcgtcatcctctctctggacttcatcctgttctgcctccggctcatgcacatttttaccatcagtaagacgctggggcccaagatcatcattgtgaagcggatgatgaaggacgtcttcttcttcctcttcctgctggctgtgtgggtggtgtccttcggggtggccaagcaggccatcctcatccacaacgagcgccgggtggactggctgttccgaggggccgtctaccactcctacctcaccatcttcgggcagatcccgggctacatcgacggtgtgaacttcaacccggagcactgcagccccaatggcaccgacccctacaagcctaagtgccccgagagcgacgcgacgcagcagaggccggccttccctgagtggctgacggtcctcctactctgcctctacctgctcttcaccaacatcctgctgctcaacctcctcatcgccatgttcaactacaccttccagcaggtgcaggagcacacggaccagatttggaagttccagcgccatgacctgatcgaggagtaccacggccgccccgccgcgccgccccccttcatcctcctcagccacctgcagctcttcatcaagagggtggtcctgaagactccggccaagaggcacaagcagctcaagaacaagctggagaagaacgaggaggcggccctgctatcctgggagatctacctgaaggagaactacctccagaaccgacagttccagcaaaagcagcggcccgagcagaagatcgaggacatcagcaataaggcagggctggagctttgggggtcaaggacaagctctgtctgcaaatctagacagttcctgcatctcacggtggtggaatcacgcgggtccgagaaggctccagtaaccaccctcgcatgtttgcaaggctgtctgggaaaacatggcagggttgacgccatggtggacctgctggacctggacccactgaagaggtcgggctccatggagcagaggttggcctccctggaggagcaggtggcccagacagcccaagccctgcactggatcgtgaggacgctgcgggccagcggcttcagctcggaggcggacgtccccactctggcctcccagaaggccgcggaggagccggatgctgagccgggaggcaggaagaagacggaggagccgggcgacagctaccacgtgaatgcccggcacctcctctaccccaactgccctgtcacgcgcttccccgtgcccaacgagaaggtgccctgggagacggagttcctgatctatgacccacccttttacacggcagagaggaaggacgcggccgccatggaccccatgggagacaccctggagccactgtccacgatccagtacaacgtggtggatggcctgagggaccgccggagcttccacgggccgtacacagtgcaggccgggttgcccctgaaccccatgggccgcacaggactgcgtgggcgcgggagcctcagctgcttcggacccaaccacacgctgtaccccatggtcacgcggtggaggcggaacgaggatggagccatctgcaggaagagcataaagaagatgctggaagtgctggtggtgaagctccctctctccgagcactgggccctgcctgggggctcccgggagccaggggagatgctacctcggaagctgaagcggatcctccggcaggagcactggccgtcttttgaaaacttgctgaagtgcggcatggaggtgtacaaaggctacatggatgacccgaggaacacggacaatgcctggatcgagacggtggccgtcagcgtccacttccaggaccagaatgacgtggagctgaacaggctgaactctaacctgcacgcctgcgactcgggggcctccatccgatggcaggtggtggacaggcgcatcccactctatgcgaaccacaagaccctcctccagaaggcagccgctgagttcggggctcactactgactgtgccctcaggctgggcggctccagtccatagacgttccccccagaaaccagggcttctctctcctgagcctggccaggactcaggctgttcctgggccctgcacatgatggggtttggtggacccagtgcccctcacggctgccgcaagtctgctgcagatgacctcatgaactggaaggggtcaaggtgacccgggaggagagctcaagacagggcacaggctactcagagctgaggggcccctgggacccttggccatcaggcgaggggctgggcctgtgcagctgggcccttggccagagtccactcccttcctggctgtgtcaccccgagcagctcatccaccatggaggtcattggcctgaggcaagttccccggagagtcggggtcccctgtggccccctcaggcctatgtctgtgaggaaggggccctgccactctccccaagagggcctccatgtttcgaggtgcctcaacatggagccttgcctggcctgggctaggggcactgtctgaactcctgactgtcaggataaactccgtgggggtacaggagcccagacaaagcccaggcctgtcaagagacgcagagggcccctgccagggttggccccagggaccctgggacgaggctgcagaagctctccctccctactccctgggagccacgtgctggccatgtggccagggacggcatgagcaggaggcggggacgtgggggccttctggtttggtgtcaacagctcacaggagcgtgaaccatgagggccctcaggaggggaacgtggtaaaacccaagacattaaatctgccatctcaggc

SEQ ID NO: 2 - protein, 1553 aa, Homo sapiens
MEPSALRKAGSEQEEGFEGLPRRVTDLGMVSNLRRSNSSLFKSWRLQCPFGNNDKQESLSSWIPENIKKKECVYFVESSKLSDAGKVVCQCGYTHEQHLEEATKPHTFQGTQWDPKKHVQEMPTDAFGDIVFTGLSQKVKKYVRVSQDTPSSVIYHLMTQHWGLDVPNLLISVTGGAKNFNMKPRLKSIFRRGLVKVAQTTGAWIITGGSHTGVMKQVGEAVRDFSLSSSYKEGELITIGVATWGTVHRREGLIHPTGSFPAEYILDEDGQGNLTCLDSNHSHFILVDDGTHGQYGVEIPLRTRLEKFISEQTKERGGVAIKIPIVCVVLEGGPGTLHTIDNATTNGTPCVVVEGSGRVADVIAQVANLPVSDITISLIQQKLSVFFQEMFETFTESRIVEWTKKIQDIVRRRQLLTVFREGKDGQQDVDVAILQALLKASRSQDHFGHENWDHQLKLAVAWNRVDIARSEIFMDEWQWKPSDLHPTMTAALISNKPEFVKLFLENGVQLKEFVTWDTLLYLYENLDPSCLFHSKLQKVLVEDPERPACAPAAPRLQMHHVAQVLRELLGDFTQPLYPRPRHNDRLRLLLPVPHVKLNVQGVSLRSLYKRSSGHVTFTMDPIRDLLIWAIVQNRRELAGIIWAQSQDCIAAALACSKILKELSKEEEDTDSSEEMLALAEEYEHRAIGVFTECYRKDEERAQKLLTRVSEAWGKTTCLQLALEAKDMKFVSHGGIQAFLTKVWWGQLSVDNGLWRVTLCMLAFPLLLTGLISFREKRLQDVGTPAARARAFFTAPVVVFHLNILSYFAFLCLFAYVLMVDFQPVPSWCECAIYLWLFSLVCEEMRQLFYDPDECGLMKKAALYFSDFWNKLDVGAILLFVAGLTCRLIPATLYPGRVILSLDFILFCLRLMHIFTISKTLGPKIIIVKRMMKDVFFFLFLLAVWVVSFGVAKQAILIHNERRVDWLFRGAVYHSYLTIFGQIPGYIDGVNFNPEHCSPNGTDPYKPKCPESDATQQRPAFPEWLTVLLLCLYLLFTNILLLNLLIAMFNYTFQQVQEHTDQIWKFQRHDLIEEYHGRPAAPPPFILLSHLQLFIKRVVLKTPAKRHKQLKNKLEKNEEAALLSWEIYLKENYLQNRQFQQKQRPEQKIEDISNKAGLELWGSRTSSVCKSRQFLHLTVVESRGSEKAPVTTLACLQGCLGKHGRVDAMVDLLDLDPLKRSGSMEQRLASLEEQVAQTAQALHWIVRTLRASGFSSEADVPTLASQKAAEEPDAEPGGRKKTEEPGDSYHVNARHLLYPNCPVTRFPVPNEKVPWETEFLIYDPPFYTAERKDAAAMDPMGDTLEPLSTIQYNVVDGLRDRRSFHGPYTVQAGLPLNPMGRTGLRGRGSLSCFGPNHTLYPMVTRWRRNEDGAICRKSIKKMLEVLVVKLPLSEHWALPGGSREPGEMLPRKLKRILRQEHWPSFENLLKCGMEVYKGYMDDPRNTDNAWIETVAVSVHFQDQNDVELNRLNSNLHACDSGASIRWQVVDRRIPLYANHKTLLQKAAAEFGAHY

SEQ ID NO: 3 - protein, 17 aa, Homo sapiens
EEEDTDSSEEMLALAEE

SEQ ID NO: 4 - protein, 27 aa, synthetic construct
YGRKKRRQRRREEDTDSSEEMLALAEE

SEQ ID NO: 5 - protein, 16 aa, Homo sapiens
QFQKNKLRINRQHSYD

SEQ ID NO: 6 - protein, 12 aa, Homo sapiens
QKNKLRINRQHS

SEQ ID NO: 7 - protein, 15 aa, Homo sapiens
AQKKNRNKLRRQHSY

SEQ ID NO: 8 - protein, 11 aa, Homo sapiens
KKNRNKLRRQH

SEQ ID NO: 9 - protein, 4 aa, Homo sapiens
DKKR

SEQ ID NO: 10 - protein, 6 aa, synthetic construct
HHHHHH

SEQ ID NO: 11 - protein, 25 aa, synthetic construct
YGRKKRRQRRRVILLKDHTLEYPVF

SEQ ID NO: 12 - DNA, 32 bp, synthetic construct
  source 1..32: mol_type = other DNA
ctcgaattctgaaggagaactacctccagaac

SEQ ID NO: 13 - DNA, 34 bp, synthetic construct
  source 1..34: mol_type = other DNA
gatctagattaggtcttgtggttcgcatagagtg

SEQ ID NO: 14 - DNA, 28 bp, synthetic construct
  source 1..28: mol_type = other DNA
cggaattccgacactcttctactggaag

SEQ ID NO: 15 - DNA, 35 bp, synthetic construct
  source 1..35: mol_type = other DNA
tgctctagagcttaaacatcagattcgatactagg

SEQ ID NO: 16 - DNA, 30 bp, synthetic construct
  source 1..30: mol_type = other DNA
cggaattccgtcatcaccttcatctgtgag

SEQ ID NO: 17 - DNA, 31 bp, synthetic construct
  source 1..31: mol_type = other DNA
tgctctagagcaccttaacctctctctcttc

SEQ ID NO: 18 - DNA, 21 bp, synthetic construct
  source 1..21: mol_type = other DNA
caaggacatgtagtttgtgtc

SEQ ID NO: 19 - DNA, 24 bp, synthetic construct
  source 1..24: mol_type = other DNA
gacacaaactacatcatgtccttg

SEQ ID NO: 20 - DNA, 21 bp, synthetic construct
  source 1..21: mol_type = other DNA
tggcgctggcgtagtagtatg

SEQ ID NO: 21 - DNA, 21 bp, synthetic construct
  source 1..21: mol_type = other DNA
tcatactactacgccagcgcc

SEQ ID NO: 22 - DNA, 24 bp, synthetic construct
  source 1..24: mol_type = other DNA
atttgggccattgtctagaaccgt

SEQ ID NO: 23 - DNA, 25 bp, synthetic construct
  source 1..25: mol_type = other DNA
acggttctagacaatggcccaaatg

SEQ ID NO: 24 - DNA, 20 bp, synthetic construct
  source 1..20: mol_type = other DNA
tgctgggggaattcacgcag

SEQ ID NO: 25 - DNA, 20 bp, synthetic construct
  source 1..20: mol_type = other DNA
tgcgtgaattcccccagcag

SEQ ID NO: 26 - DNA, 26 bp, synthetic construct
  source 1..26: mol_type = other DNA
tgaaggaactgtcctaggaggaggag

SEQ ID NO: 27 - DNA, 26 bp, synthetic construct
  source 1..26: mol_type = other DNA
tcctcctcctaggacagttccttcag

SEQ ID NO: 28 - DNA, 42 bp, synthetic construct
  source 1..42: mol_type = other DNA
aagatcctgaaggaactgtccaagtatgagcacagagccatc

SEQ ID NO: 29 - DNA, 42 bp, synthetic construct
  source 1..42: mol_type = other DNA
gatggctctgtgctcatacttggacagttccttcaggatctt

SEQ ID NO: 30 - DNA, 33 bp, synthetic construct
  source 1..33: mol_type = other DNA
aaggaactgtccaagcagcaggaggacacggac

SEQ ID NO: 31 - DNA, 33 bp, synthetic construct
  source 1..33: mol_type = other DNA
tccgtgtcctcctgctgcttggacagttccttc

SEQ ID NO: 32 - DNA, 29 bp, synthetic construct
  source 1..29: mol_type = other DNA
acacggacagctcgcagcagatgctggcg

SEQ ID NO: 33 - DNA, 30 bp, synthetic construct
  source 1..30: mol_type = other DNA
cgccagcatctgctgcgagctgtccgtgtc

SEQ ID NO: 34 - DNA, 30 bp, synthetic construct
  source 1..30: mol_type = other DNA
tggcgctggcgcagcagtatgagcacagag

SEQ ID NO: 35 - DNA, 30 bp, synthetic construct
  source 1..30: mol_type = other DNA
tctgtgctcatactgctgcgccagcgccag

SEQ ID NO: 36 - DNA, 27 bp, synthetic construct
  source 1..27: mol_type = other DNA
accttcatctggtagcacctcttctac

SEQ ID NO: 37 - DNA, 27 bp, synthetic construct
  source 1..27: mol_type = other DNA
tagaagaggtgctaccagatgaaggtg

SEQ ID NO: 38 - DNA, 29 bp, synthetic construct
  source 1..29: mol_type = other DNA
accttcatctgttagcatctgttctattg

SEQ ID NO: 39 - DNA, 29 bp, synthetic construct
  source 1..29: mol_type = other DNA
aatagaacagatgctaacagatgaaggtg

SEQ ID NO: 40 - DNA, 26 bp, synthetic construct
  source 1..26: mol_type = other DNA
tatgataacattctggacaaacccag

SEQ ID NO: 41 - DNA, 18 bp, synthetic construct
  source 1..18: mol_type = other DNA
gaactggagggcgttgtt

SEQ ID NO: 42 - DNA, 18 bp, synthetic construct
  source 1..18: mol_type = other DNA
tcctacgacaccttcgtg

SEQ ID NO: 43 - DNA, 21 bp, synthetic construct
  source 1..21: mol_type = other DNA
ctgagccttggaattagtcgg

SEQ ID NO: 44 - DNA, 32 bp, synthetic construct
  source 1..32: mol_type = other DNA
atatgcggccgctcaggccatgtgaccttcac

SEQ ID NO: 45 - DNA, 34 bp, synthetic construct
  source 1..34: mol_type = other DNA
atatggtaccttacttcatgtccttggcctccag

SEQ ID NO: 46 - DNA, 32 bp, synthetic construct
  source 1..32: mol_type = other DNA
atatgcggccgctctcctttcaagtgtgatgc

SEQ ID NO: 47 - DNA, 34 bp, synthetic construct
  source 1..34: mol_type = other DNA
atatggtaccttagcttttgttccccaagagttt

SEQ ID NO: 48 - DNA, 30 bp, synthetic construct
  source 1..30: mol_type = other DNA
atatgcggccgcgaggcctgtaagaaggct

SEQ ID NO: 49 - DNA, 32 bp, synthetic construct
  source 1..32: mol_type = other DNA
atatggtaccttatgaggacttgttggcaaag

SEQ ID NO: 50 - DNA, 20 bp, synthetic construct
  source 1..20: mol_type = other DNA
gatatctcacgtactgacgg

SEQ ID NO: 51 - DNA, 20 bp, synthetic construct
  source 1..20: mol_type = other DNA
tgaccagagtcatggttagc

SEQ ID NO: 52 - DNA, 23 bp, synthetic construct
  source 1..23: mol_type = other DNA
ggctctgcctcatccccagaatc

SEQ ID NO: 53 - DNA, 23 bp, synthetic construct
  source 1..23: mol_type = other DNA
ccggatacagatgcaggatgctg

SEQ ID NO: 54 - DNA, 25 bp, synthetic construct
  source 1..25: mol_type = other DNA
ctgaaggtcctgagtttgaatccca

SEQ ID NO: 55 - DNA, 24 bp, synthetic construct
  source 1..24: mol_type = other DNA
cttgggttgcagtcatatgcaggc

SEQ ID NO: 56 - DNA, 24 bp, synthetic construct
  source 1..24: mol_type = other DNA
gccctcaccatccgcttcacgatg

SEQ ID NO: 57 - protein, 43 aa, Homo sapiens
LVACKLYKAMAHESSESDLVDDISQDLDNNSKDFGQLALELLD

SEQ ID NO: 58 - protein, 38 aa, Homo sapiens
ACSKILKELSKEEEDTDSSEEMLALAEEYEHRAIGVFT

SEQ ID NO: 59 - protein, 42 aa, Homo sapiens
VACKLCKAMAHEASENDMVDDISQELNHNSRDFGQLAVELLD

SEQ ID NO: 60 - protein, 38 aa, Homo sapiens
GACLLLRVMARLEPDAEEAARRKDLAFKFEGMGVDLFG

SEQ ID NO: 61 - protein, 36 aa, Homo sapiens
AACKILKEMSHLETEAEAARATREAKYERLALDLFS

SEQ ID NO: 62 - protein, 42 aa, Homo sapiens
IACILYRAMAHEAKESHMVDDASEELKNYSKQFGQLALDLLE

SEQ ID NO: 63 - protein, 42 aa, Homo sapiens
VACKIYRSMAYEAKQSDLVDDTSEELKQYSNDFGQLAVELLE

SEQ ID NO: 64 - protein, 38 aa, Homo sapiens
GASKLLKTLAKVKNDINAAGESEELANEYETRAVELFT

SEQ ID NO: 65 - protein, 38 aa, Mus musculus
ACSKILKELSKEEEDTDSSEEMLALADEFEHRAIGVFT

SEQ ID NO: 66 - protein, 38 aa, Anolis carolinensis
ACSKILKELSKEETDTDSTEEMLALAEEYEHRAIGVFT

SEQ ID NO: 67 - protein, 38 aa, Gallus gallus
ACSKILKELAKEEEDTDTTDEMLALAEQYEHKAIGVFT

SEQ ID NO: 68 - protein, 38 aa, Xenopus laevis
ACSKLLKELSKEEEDTDTSEEMLALAEEFEQRAIGIFT

SEQ ID NO: 69 - protein, 39 aa, Danio rerio
ASKILRKLAQESGEDDSEEATEMLELANHYEKQAIGVFS

SEQ ID NO: 70 - protein, 37 aa, Drosophila melanogaster
NTCAHIAAMQGSVKVIEELMKFDRSGVISARNKLTDA

SEQ ID NO: 71 - protein, 6 aa, unidentified
QNNALQ

SEQ ID NO: 72 - protein, 13 aa, unidentified
QKNKLRINRQHSY

SEQ ID NO: 73 - protein, 171 aa, unidentified
NILDKPREIDLSRPSRSISLKDRERLLEGNLYGSLFSVPSSKLLGNKSSLFPQGLEDSKRSKSLLPDHASDNPFLHTYGDDQRLVIGRCPSDPYKHSLPSQAVNDSYLRSSLRSTASYCSRDSRGHSDVYISEHVMPYAANKNTMYSTPRVLNSCSNRRVYKKMPSIESDV

SEQ ID NO: 74 - protein, 10 aa, unidentified
YPQSPTNSKA

SEQ ID NO: 75 - protein, 13 aa, unidentified
KKNRNKLRRQHSY

SEQ ID NO: 76 - protein, 177 aa, unidentified
TFVDLQKEEAALAPRSVSLKDKGRFMDGSPYAHMFEMPAGESSFANKSSVPTAGHHHNNPGSGYMLSKSLYPDRVTQNPFIPTFGDDQCLLHGSKSYFFRQPTVAGASKTRPDFRALVTNKPVVSALHGAVPGRFQKDICIGNQSNPCVPNNKNPRAFNGSSNGHVYEKLSSIESDV

SEQ ID NO: 77 - protein, 41 aa, Danio rerio
PDGRGTGGGSVDRAFSPFLPQPDASFTAYLPQSDQEGDGLI

SEQ ID NO: 78 - protein, 14 aa, Danio rerio
HYQNEGVIIRQHSY

SEQ ID NO: 79 - protein, 33 aa, Danio rerio
NLPQKSSSRRVSLQDNTPYANIPSATLMPDKPP

SEQ ID NO: 80 - protein, 23 aa, Xenopus laevis
EAANSMHSEDFYEHNWLENNALH

SEQ ID NO: 81 - protein, 14 aa, Xenopus laevis
QKKNKLRINRQHSC

SEQ ID NO: 82 - protein, 35 aa, Xenopus laevis
NINKPREHDLGRPPRSLSLKEKERYIQENPFAKFV

SEQ ID NO: 83 - protein, 24 aa, Anolis carolinensis
DTAAETVPRAESYRHNWVQNNQTQ

SEQ ID NO: 84 - protein, 14 aa, Anolis carolinensis
PKKSKLKIGRQRSF

SEQ ID NO: 85 - protein, 45 aa, Anolis carolinensis
NLFEKAREADAAARPSRSISLREREKLLEGGGGGGGGGLFASRFN

SEQ ID NO: 86 - protein, 23 aa, Gallus gallus
DTTNLSLPEEIYGHSWSQNNALQ

SEQ ID NO: 87 - protein, 14 aa, Gallus gallus
HKKNKLRISRQHSF

SEQ ID NO: 88 - protein, 36 aa, Gallus gallus
NIADKSREIDLGRPSRSISLKDKEKFLQSSPYASMF

SEQ ID NO: 89 - protein, 23 aa, Mus musculus
ETGNPATREEAYQQDWSQNNALQ

SEQ ID NO: 90 - protein, 13 aa, Mus musculus
QKNKLKINRQHSY

SEQ ID NO: 91 - protein, 36 aa, Mus musculus
NILDKPREIDLSRPSRSISLKDRERLLEGNLYGSLF

SEQ ID NO: 92 - protein, 23 aa, Homo sapiens
ETGNPATGEQVYQQDWAQNNALQ

SEQ ID NO: 93 - protein, 13 aa, Homo sapiens
QKNKLRISRQHSY

SEQ ID NO: 94 - protein, 36 aa, Homo sapiens
NIVDKPRELDLSRPSRSISLKDRERLLEGNFYGSLF

SEQ ID NO: 95 - protein, 4 aa, Danio rerio
LRDF

SEQ ID NO: 96 - protein, 8 aa, Danio rerio
VDQFRAKE

SEQ ID NO: 97 - protein, 18 aa, Xenopus laevis
RPIQAPNSKYPQSPNGKA

SEQ ID NO: 98 - protein, 13 aa, Xenopus laevis
KRNRSKLHRQHSY

SEQ ID NO: 99 - protein, 8 aa, Xenopus laevis
TFVDLQKE

SEQ ID NO: 100 - protein, 27 aa, Anolis carolinensis
PSAPVPVATSTSTTKYPQSPSNSASKV

SEQ ID NO: 101 - protein, 13 aa, Anolis carolinensis
KKNRNKLRRQHSY

SEQ ID NO: 102 - protein, 8 aa, Anolis carolinensis
TFVDLQKE

SEQ ID NO: 103 - protein, 28 aa, Gallus gallus
PPAPVPVATSATSSSKYPQSPSNSASKV

SEQ ID NO: 104 - protein, 13 aa, Gallus gallus
KKNRNKLRRQHSY

SEQ ID NO: 105 - protein, 8 aa, Gallus gallus
TFVDLQKD

SEQ ID NO: 106 - protein, 25 aa, Mus musculus
PAAPVAVSSNASTTKYPQSPTNSKA

SEQ ID NO: 107 - protein, 13 aa, Mus musculus
KKNRNKLRRQHSY

SEQ ID NO: 108 - protein, 8 aa, Mus musculus
TFVDLQKE

SEQ ID NO: 109 - protein, 25 aa, Homo sapiens
PAAPVAVTSNASTTKYPQSPTNSKA

SEQ ID NO: 110 - protein, 13 aa, Homo sapiens
KKNRNKLRRQHSY

SEQ ID NO: 111 - protein, 8 aa, Homo sapiens
TFVDLQKE

SEQ ID NO: 112 - DNA, 24 bp, synthetic construct
  source 1..24: mol_type = other DNA
gccacacgcgtcaccttaatatgc

(19) United States
(12) Patent Application Publication
(10) Pub. No.: US 2025/0235501 A1
(43) Pub. Date: Jul. 24, 2025

(54) Methods and Compositions for Treating or Preventing Neurological Injury or Neurological Disorders

(71) Applicant: University of Connecticut, Farmington, CT (US)

(72) Inventors: Lixia Yue, Unionville, CT (US); Pengyu Zong, Unionville, CT (US); Jianlin Feng, Unionville, CT (US)

(73) Assignee: University of Connecticut, Farmington, CT (US)

(21) Appl. No.: 18/419,215

(22) Filed: Jan. 22, 2024

(51) Int. Cl. A61K 38/16; A61K 45/06; A61P 9/10; G01N 33/50

(52) U.S. Cl. CPC A61K 38/16; A61K 45/06; A61P 9/10; G01N 33/5035; G01N 2333/705

(57) ABSTRACT

The present invention provides methods and compositions for treating or preventing neurological injury or neurological disorders in a subject in need thereof by administration of an agent that inhibits the activity of TRPM2, e.g., by inhibiting the interaction between TRPM2 and its binding protein, NMDAR. The present invention also provides a peptide-based therapeutic agent, TAT-EE3, and its use in treating or preventing neurological injury or neurological disorders.

### RELATED APPLICATIONS

This application is a 35 § U.S.C. 111 (a) continuation application which claims the benefit of priority to International Patent Application No. PCT/US2022/038511, filed on Jul. 27, 2022, which, in turn, claims the benefit of priority to U.S. Provisional Application No. 63/226,693, filed on Jul. 28, 2021. The entire contents of each of the foregoing application are incorporated herein by reference.

### STATEMENT OF GOVERNMENT INTEREST

This invention was made with government support under grant number HL143750 from the National Institutes of Health. The government has certain rights in the invention.

### SEQUENCE LISTING

The instant application contains a Sequence Listing which has been submitted electronically in XML format and is hereby incorporated by reference in its entirety. Said XML copy, created Sep. 9, 2022, is named 98121_00356_SL.xml and is 106,903 bytes in size.

### BACKGROUND OF THE INVENTION

Approximately 15 million people worldwide suffer a stroke each year, resulting in death or sensorimotor and other defects. Indeed, stroke remains the third most common cause of death in the industrialized world behind heart disease and cancer. There are two main forms of stroke: ischemic stoke, caused by a blood clot that blocks or prevents the flow of blood; and hemorrhagic stroke, caused by bleeding into or around the brain. Ischemic stroke accounts for approximately 80-86% of all stroke cases.

Neuronal death is a hallmark of ischemic stroke. Numerous factors are involved in neuronal damage during ischemic stroke, among which Ca2+ overload plays a key role in neurotoxicity (Granzotto, A., et al, (2020). Frontiers in molecular neuroscience 13, 600089). Ca2+ overload caused by excitotoxic mechanisms through NMDA receptor (NMDAR) activation and non-excitotoxic Ca2+ entry mechanisms triggers a series of downstream signaling cascades, including reactive oxygen species (ROS) or reactive nitrogen species (RNS) generation, mitochondrial dysfunction, metabolic impairment, and activation of necrosis and apoptosis cascade, and ultimately leads to neuronal death (Choi, D. W. (2020). Frontiers in neuroscience 14, 579953). Since it was first discovered fifty years ago (Olney, J. W. (1969). Science 164, 719-21), excitotoxicity caused by NMDAR mediated Ca2+ overload has been the center of extensive research for understanding the underlying mechanisms and for developing effective therapeutics for ischemic stroke. However, the development of stroke drugs by antagonizing NMDARs has been characterized by success in animal studies but subsequent failure in clinical trials (Sena, E., et al., (2007). Trends in neurosciences 30, 433-39).

The lack of clinical success with excitotoxic NMDAR antagonists prompted a shift of the focus of stroke neuroprotection research towards the identification of downstream intracellular signaling pathways triggered by NMDARs (Wu, Q. J., and Tymianski, M. (2018). Mol Brain 11, 15), and the investigation of subtype-dependent (Ge, Y., et al., (2020). Trends in molecular medicine 26, 533-51) as well as localization-dependent excitotoxic effects of NMDARs (Hardingham, G. E., and Bading, H. (2010). Nature reviews Neuroscience 11, 682-96). Over a third of surface NMDARs are located extrasynaptically (Petit-Pedrol, M., and Groc, L. (2021). The Journal of cell biology 220), which preferentially leads to neurotoxicity and cell death upon activation, whereas activation of synaptic NMDARs promotes surviving mechanism, likely through activation of differential signaling pathways triggered by intracellular Ca2+ (Bading, H. (2013). Nature reviews Neuroscience 14, 593-08). Moreover, the disappointing clinical trial outcome of NMDAR antagonists for stroke treatment also promoted a divergent focus on investigating non-excitotoxic Ca2+ permeable channels as therapeutic targets (Tymianski, M. (2011). Nature neuroscience 14, 1369-73). TRPM2 is a target that has been implicated in the pathogenesis of ischemic stroke.

TRPM2 was discovered as an oxidative stress activated Ca2+-permeable non-selective cation channel (Hara, Y., et al. (2002). Molecular cell 9, 163-73; Perraud, A. L., et al. (2001). Nature 411, 595-99; Sano, Y., et al., (2001). Science 293, 1327-30), belonging to the superfamily of transient receptor potential (TRP), melastatin subfamily (Clapham, D. E. (2003). Nature 426, 517-24; Montell, C., et al., (2002). Cell 108, 595-98). TRPM2 is ubiquitously expressed in various cell types and most abundantly expressed in the brain (Fonfria, E., et al., (2006). Journal of receptor and signal transduction research 26, 159-78). In response to oxidative stress stimuli, TRPM2-mediated Ca2+ influx leads to cell death of a various cell types including neuron (Belrose, J. C., and Jackson, M. F. (2018). Acta pharmacologica Sinica 39, 722-32; Mai, C., et al., (2020). Journal of cellular and molecular medicine 24, 4-12).

Given the complexity of deleterious effects caused by both excitotoxic Ca2+ and non-excitotoxic Ca2+ signaling pathways during ischemic stroke, mitigating ischemic injury via these pathways proves to be challenging.

Current pharmacotherapy for ischemic stroke is limited. Administration of thrombolytic agents, such as tissue plasminogen activator (tPA), which dissolve blood clots and thus restore blood flow to affected regions, has limited applicability. In particular, administration of tPA is only effective if given within three hours from the time of stroke onset. This three-hour therapeutic window must include time for diagnosis, as the use of tPA for treatment of stroke is limited to that of ischemic stroke and cannot be administered to a patient having had a hemorrhagic stroke. The use of tPA for treatment of ischemic stroke has other limitations, including the fact that not all clinicians are adequately trained to deliver tPA, and that tPA has also been associated with extravascular deleterious effects, including hemorrhagic transformation, microvascular dysfunction, and excitotoxic neuronal damage.

Moreover, use of thrombolytic agents, such as tPA, as well as other existing stroke therapies, target only a specific subset of deleterious symptoms associated with or resulting from stroke, and therefore fail to provide a complete therapeutic approach for addressing both the immediate and long-term consequences following a stroke. Additionally, such therapies are often of high cost and have limited modes of administration.

Therefore, a need exists for a therapy that is effective in the treatment all types of stroke, i.e., both ischemic strokes and hemorrhagic strokes. There is also a need for a therapy that is effective in the treatment of stroke even when administered beyond the therapeutic window of current treatments.

### SUMMARY OF THE INVENTION

The present invention is based, at least in part, on the discovery that TRPM2 in neurons plays a key role in mediating the deleterious effects of TRPM2 in ischemic brain stroke. The inventors discovered that physical and functional coupling of TRPM2 with NMDARs leads to enhanced extrasynaptic NMDAR toxicity under oxidative stress condition. Interaction of TRPM2 with PKC□ underlies the mechanism by which TRPM2 mediates enhancement of NMDAR surface expression and functional increase of NMDAR currents. In addition, the inventors identified a specific domain of TRPM2 and a specific domain of NMDAR which are responsible for the interaction between TRPM2 and NMDAR, and designed a membrane permeable disrupting peptide TAT-EE3. It was demonstrated that uncoupling TRPM2 from NMDARs results in significant reduction of ischemic neuronal toxicity mediated by both non-excitotoxic and excitotoxic Ca2+ signaling pathways and leads to protective effects in vitro and in vivo. Specifically, administering the disrupting peptide effectively decreased the infarction size and improved the symptoms of mice undergoing middle cerebral artery occlusion, suggesting that the peptide is a promising therapeutic candidate for treating ischemic stroke in human.

Moreover, the newly identified NMDAR-binding domain of TRPM2 and/or the TRPM2-binding domain of NMDAR can be used as a drug target for developing further therapeutic candidates, such as therapeutic peptides, e.g., TAT-EE3, or small molecules, for ischemic stroke and other TRPM2-associated neurodegenerative diseases, e.g., Alzheimer diseases.

Accordingly, the present invention provides, in one aspect, a method for treating or preventing neurological injury or neurological disorder in a subject comprising administering to the subject an agent that inhibits the interaction between N-methyl-D-aspartate receptor (NMDAR) and transient receptor potential cation channel subfamily M member 2 (TRPM2).

In some embodiments, the agent targets a NMDAR-binding site on TRPM2.

In some embodiments, the NMDAR-binding site comprises residues 631 to 679 or residues 665-681 of TRPM2. In some embodiments, the NMDAR-binding site comprises an amino acid sequence of EEEDTDSSEEMLALAEE (“TRPM2-EE3”, SEQ ID NO: 3).

In some embodiments, the agent comprises a small molecule that binds to the NMDAR-binding site.

In some embodiments, the agent comprises a peptide comprising an amino acid sequence with at least 80%, 85%, 90% or 95% sequence identity to the amino acid sequence of EEEDTDSSEEMLALAEE (SEQ ID NO:3), or the amino acid sequence of

(“TAT-EE3”, SEQ ID NO: 4)
YGRKKRRQRRREEDTDSSEEMLALAEE.

In some embodiments, the peptide comprises an amino acid sequence differing by 1, 2, 3, 4, or 5 residues from the amino acid sequence of EEEDTDSSEEMLALAEE (SEQ ID NO:3) or the amino acid sequence of

(SEQ ID NO: 4)
YGRKKRRQRRREEDTDSSEEMLALAEE.

In some embodiments, the peptide comprises the amino acid sequence of

(SEQ ID NO: 4)
YGRKKRRQRRREEDTDSSEEMLALAEE.

In some embodiments, the peptide does not comprise the amino acid sequence of

(SEQ ID NO: 3)
EEEDTDSSEEMLALAEE.

In some embodiments, the agent comprises an antagonist anti-TRPM2 antibody that binds to the NMDAR-binding site.

In some embodiments, the agent comprises a mutant TRPM2 protein, wherein the mutant TRPM2 protein comprises a deletion of the NMDAR-binding site.

In some embodiments, the mutant TRPM2 protein comprises a substitution mutation at residue 674, a substitution mutation at residue 675, or substitution mutations at both residues 674 and 675 of TRPM2 protein.

In some embodiments, the agent targets a TRPM2-binding site on NMDAR. In some embodiments, the TRPM2-binding site comprises an amino acid sequence of QFQKNKLRINRQHSYD (SEQ ID NO:5), or an amino acid sequence of QKNKLRINRQHS (SEQ ID NO:6). In some embodiments, the TRPM2-binding site comprises an amino acid sequence of AQKKNRNKLRRQHSY (SEQ ID NO:7), or an amino acid sequence of KKNRNKLRRQH (SEQ ID NO:8).

In some embodiments, the agent comprises a small molecule that binds to the TRPM2-binding site.

In some embodiments, the agent comprises a peptide comprising an amino acid sequence with at least 80%, 85%, 90% or 95% sequence identity to the amino acid sequence of QFQKNKLRINRQHSYD (SEQ ID NO:5), or the amino acid sequence of

(SEQ ID NO: 6)
QKNKLRINRQHS.

In some embodiments, the peptide comprises an amino acid sequence differing by 1, 2, 3, 4, or 5 residues from the amino acid sequence of QFQKNKLRINRQHSYD (SEQ ID NO: 5), or the amino acid sequence of QKNKLRINRQHS (SEQ ID NO:6).

In some embodiments, the peptide does not comprise the amino acid sequence of QFQKNKLRINRQHSYD (SEQ ID NO:5), or the amino acid sequence of

(SEQ ID NO: 6)
QKNKLRINRQHS.

In some embodiments, the agent comprises a peptide comprising an amino acid sequence with at least 80%, 85%, 90% or 95% sequence identity to the amino acid sequence of AQKKNRNKLRRQHSY (SEQ ID NO:7), or the amino acid sequence of

(SEQ ID NO: 8)
KKNRNKLRRQH.

In some embodiments, the peptide comprises an amino acid sequence differing by 1, 2, 3, 4, or 5 residues from the amino acid sequence of AQKKNRNKLRRQHSY (SEQ ID NO: 7), or the amino acid sequence of KKNRNKLRRQH (SEQ ID NO:8).

In some embodiments, the peptide does not comprise the amino acid sequence of AQKKNRNKLRRQHSY (SEQ ID NO:7), or the amino acid sequence of

(SEQ ID NO: 8)
KKNRNKLRRQH.

In some embodiments, the agent comprises an antagonist anti-NMDAR antibody that binds to the TRPM2-binding site.

In some embodiments, the agent comprises a mutant NMDAR protein, wherein the mutant NMDAR protein comprises a deletion of the TRPM2-binding site.

In some embodiments, the neurological injury results from a brain injury. In some embodiments, the brain injury is selected from the group consisting of stroke, traumatic brain injury, cerebral palsy, acquired brain injury, anoxic brain injury, diffuse axonal brain injury, focal brain injury, subdural hematoma, brain aneurysm, and coma. In some embodiments, the brain injury is ischemic stroke, hemorrhagic stroke, or transient ischemic attack.

In some embodiments, the neurological disorder is a neurodegenerative disease. In some embodiments, the neurodegenerative disease is selected from the group consisting of Alzheimer's Disease, multiple sclerosis, HIV-associated dementia, Huntington's Disease, Parkinson's Disease, and amyotrophic lateral sclerosis.

In some embodiments, the subject is a human subject.

In one aspect, the present invention provides a peptide comprising an amino acid sequence with at least 80% or 85% sequence identity to the amino acid sequence of YGRKKRRQRRREEDTDSSEEMLALAEE (SEQ ID NO:4), or a multimer, derivative, or variant thereof.

In some embodiments, the peptide comprises an amino acid sequence with at least 90% sequence identity to the amino acid sequence of

(SEQ ID NO: 4)
YGRKKRRQRRREEDTDSSEEMLALAEE.

In some embodiments, the peptide comprises an amino acid sequence with at least 95% sequence identity to the amino acid sequence of

(SEQ ID NO: 4)
YGRKKRRQRRREEDTDSSEEMLALAEE.

In some embodiments, the peptide comprises an amino acid sequence differing by 1, 2, 3, 4, or 5 residues from the amino acid sequence of

(SEQ ID NO: 4)
YGRKKRRQRRREEDTDSSEEMLALAEE.

In some embodiments, the peptide comprises the amino acid sequence of YGRKKRRQRRREEDTDSSEEMLALAEE (SEQ ID NO:4).

In one aspect, the present invention provides a peptide comprising an amino acid sequence with at least 80% or 85% sequence identity to the amino acid sequence of EEEDTDSSEEMLALAEE (SEQ ID NO:3), or a multimer, derivative, or variant thereof.

In some embodiments, the peptide comprises an amino acid sequence with at least 90% sequence identity to the amino acid sequence of EEEDTDSSEEMLALAEE (SEQ ID NO: 3).

In some embodiments, the peptide comprises an amino acid sequence with at least 95% sequence identity to the amino acid sequence of EEEDTDSSEEMLALAEE (SEQ ID NO: 3).

In some embodiments, the peptide comprises an amino acid sequence differing by 1, 2, 3, 4, or 5 residues from the amino acid sequence of EEEDTDSSEEMLALAEE (SEQ ID NO:3).

In some embodiments, the peptide does not comprise the amino acid sequence of

(SEQ ID NO: 3)
EEEDTDSSEEMLALAEE.

In one aspect, the present invention provides a peptide comprising an amino acid sequence with at least 80% or 85% sequence identity to the amino acid sequence of QFQKNKLRINRQHSYD (SEQ ID NO:5), or the amino acid sequence of QKNKLRINRQHS (SEQ ID NO:6), or a multimers, derivative, or variant thereof.

In some embodiments, the peptide comprises an amino acid sequence with at least 90% sequence identity to the amino acid sequence of QFQKNKLRINRQHSYD (SEQ ID NO: 5), or the amino acid sequence of QKNKLRINRQHS (SEQ ID NO:6).

In some embodiments, the peptide comprises an amino acid sequence with at least 95% sequence identity to the amino acid sequence of QFQKNKLRINRQHSYD (SEQ ID NO: 5), or the amino acid sequence of QKNKLRINRQHS (SEQ ID NO:6).

In some embodiments, the peptide comprises an amino acid sequence differing by 1, 2, 3, 4, or 5 residues from the amino acid sequence of QFQKNKLRINRQHSYD (SEQ ID NO: 5), or the amino acid sequence of QKNKLRINRQHS (SEQ ID NO:6).

In some embodiments, the peptide does not comprise the amino acid sequence of QFQKNKLRINRQHSYD (SEQ ID NO:5), or the amino acid sequence of

(SEQ ID NO: 6)
QKNKLRINRQHS.

In one aspect, the present invention provides a peptide comprising an amino acid sequence with at least 80% or 85% sequence identity to the amino acid sequence of AQKKNRNKLRRQHSY (SEQ ID NO:7), or the amino acid sequence of KKNRNKLRRQH (SEQ ID NO:8), or a multimers, derivative, or variant thereof.

In some embodiments, the peptide comprises an amino acid sequence with at least 90% sequence identity to the amino acid sequence of AQKKNRNKLRRQHSY (SEQ ID NO: 7), or the amino acid sequence of KKNRNKLRRQH (SEQ ID NO:8).

In some embodiments, the peptide comprises an amino acid sequence with at least 95% sequence identity to the amino acid sequence of AQKKNRNKLRRQHSY (SEQ ID NO: 7), or the amino acid sequence of KKNRNKLRRQH (SEQ ID NO:8).

In some embodiments, the peptide comprises an amino acid sequence differing by 1, 2, 3, 4, or 5 residues from the amino acid sequence of AQKKNRNKLRRQHSY (SEQ ID NO: 7), or the amino acid sequence of KKNRNKLRRQH (SEQ ID NO:8).

In some embodiments, the peptide does not comprise the amino acid sequence of AQKKNRNKLRRQHSY (SEQ ID NO:7), or the amino acid sequence of

(SEQ ID NO: 8)
KKNRNKLRRQH.

In some embodiments, the peptide is isolated from a cell.

In some embodiments, the peptide is a synthetic peptide.

In some embodiments, the peptide inhibits interaction between NMDAR to TRPM2.

In some embodiments, the peptide inhibits the non-excitotoxic calcium signaling pathway mediated by TRPM2.

In some embodiments, the peptide inhibits the excitotoxic calcium signaling pathway mediated by NMDAR.

In some embodiments, the peptide decreases the NMDAR surface expression in neurons.

In some embodiments, the peptide decreases the NMDAR current in neurons.

In some embodiments, the peptide reduces mitochondrial membrane depolarization induced by ischemic injury in neurons.

In some embodiments, the peptide prevents neuronal death induced by ischemic injury.

In some embodiments, the peptide is a cell permeable peptide.

In one aspect, the present invention provides a nucleic acid molecule encoding the isolated peptide of the invention.

In another aspect, the present invention provides an expression vector comprising a nucleic acid molecule operably linked to a control sequence for the expression of the peptide of the invention.

In yet another aspect, the present invention provides a host cell comprising the expression vector of the invention.

In one aspect, the present invention provides a pharmaceutical composition comprising the peptide of the invention, and at least one pharmaceutically acceptable excipient.

In another aspect, the present invention provides a method for treating or preventing neurological injury or neurological disorder in a subject comprising administering to the subject the peptide or the pharmaceutical composition of the invention.

In some embodiments, administering to the subject the agents, the peptide or the pharmaceutical composition inhibits the interaction between TRPM2 and NMDAR.

In some embodiments, administering to the subject the agent, the peptide or the pharmaceutical composition inhibits the calcium signaling pathway mediated by TRPM2.

In some embodiments, administering to the subject the agent, the peptide or the pharmaceutical composition inhibits the excitotoxic calcium signaling pathway mediated by NMDAR.

In some embodiments, administering to the subject the agent, the peptide or the pharmaceutical composition decreases the NMDAR surface expression in neurons.

In some embodiments, administering to the subject the agent, the peptide or the pharmaceutical composition decreases the NMDAR current in neurons.

In some embodiments, administering to the subject the agent, the peptide or the pharmaceutical composition reduces mitochondrial membrane depolarization induced by ischemic injury in neurons.

In some embodiments, administering to the subject the agent, the peptide or the pharmaceutical composition prevents neuronal death induced by ischemic injury.

In some embodiments, administering to the subject the agent, the peptide or the pharmaceutical composition reduces infarct volume and/or improves neurological behavior score.

In some embodiments, the neurological injury results from a brain injury. In some embodiments, the brain injury is selected from the group consisting of stroke, traumatic brain injury, cerebral palsy, acquired brain injury, anoxic brain injury, diffuse axonal brain injury, focal brain injury, subdural hematoma, brain aneurysm, and coma.

In some embodiments, the brain injury is ischemic stroke, hemorrhagic stroke, or transient ischemic attack.

In some embodiments, the neurological disorder is a neurodegenerative disease.

In some embodiments, the neurodegenerative disease is selected from the group consisting of Alzheimer's Disease, multiple sclerosis, HIV-associated dementia, Huntington's Disease, Parkinson's Disease, and amyotrophic lateral sclerosis.

In some embodiments, the subject is a human subject.

In some embodiments, the method further comprises administering to the subject an additional therapeutic agent.

In some embodiments, the additional therapeutic agent is an anticoagulant or clot-dissolving medicine, an ACE inhibitor, a blood thinner, or a statin.

In some embodiments, the additional therapeutic agent is selected from aspirin, clopidogrel, tissue plasminogen activator (tPA), lisinopril, warfarin, heparin, apixaban, atorvastatin, rosuvastatin, irbesartan, and alteplase.

In one aspect, the present invention provides a method for identifying a compound useful for treating or preventing neurological injury or neurological disorder in a subject in need thereof. The method comprises a) providing a test compound, b) determining the effect of the test compound on the interaction between TRPM2 and NMDAR, and c) selecting a compound that inhibits or reverses the interaction between TRPM2 and NMDAR, thereby identifying a compound useful for treating or preventing neurological injury or neurological disorder in the subject.

In some embodiments, the compound binds to a NMDAR-binding site on TRPM2. In some embodiments, the NMDAR-binding site comprises residues 631 to 679 or residues 665-681 of TRPM2. In some embodiments, the NMDAR-binding site comprises an amino acid sequence of EEEDTDSSEEMLALAEE (SEQ ID NO:3).

In some embodiments, the compound binds to a TRPM2-binding site on NMDAR. In some embodiments, the TRPM2-binding site comprises an amino acid sequence of QFQKNKLRINRQHSYD (SEQ ID NO:5), or an amino acid sequence of QKNKLRINRQHS (SEQ ID NO:6). In some embodiments, the TRPM2-binding site comprises an amino acid sequence of AQKKNRNKLRRQHSY (SEQ ID NO:7), or an amino acid sequence of KKNRNKLRRQH (SEQ ID NO:8).

The present invention is illustrated by the following drawings and detailed description, which do not limit the scope of the invention described in the claims.

### BRIEF DESCRIPTION OF DRAWINGS

FIGS. 1A-1Z show neuron-specific Trpm2 knockout protects brain against ischemic damage via reducing excitotoxicity during ischemia stroke.

FIGS. 1A-1C show global Trpm2 deletion (gM2KO) reduces infarct volume and improves neurological deficit (ND) score. FIG. 1A shows representative images of TTC staining of slices at 1 mm thickness from brains of wild-type (WT) and gM2KO mice 24 h after sham or MCAO surgery. FIG. 1B shows the mean infarct volume after MCAO from 12 WT and 15 gM2KO brains (***, p<0.001; ANOVA, Bonferroni's test, mean±SEM). FIG. 1C shows the average ND score 24 h after MCAO from 12 WT and 15 gM2KO mice (***: p<0.001; ANOVA, Bonferroni's test; mean±SEM).

FIGS. 1D-IF show neuron-specific Trpm2 deletion by nestin Cre (Cre+: nM2KO for simplicity) produces similar protective effects to that of gM2KO for ischemic stroke as shown in FIGS. 1A-1C. FIG. 1D shows representative images of TTC staining of Cre+ Trpm2 knockout mice (nM2KO) and Cre-control littermates (WT: for simplicity) 24 h after sham or MCAO surgery. FIG. 1E shows the average infarct volume after MCAO from 15 WT and 15 nM2KO mouse brains (***, p<0.001; ANOVA, Bonferroni's t-test, mean±SEM). FIG. 1F shows the mean ND score of the 15 nM2KO and WT mice 24 h after MCAO.

FIGS. 1G-1L show the neuronal death evaluated by Tunnel staining of brain sections from gM2KO versus WT mice (FIGS. 1G-1I) and nM2KO versus Cre-control WT littermates. FIGS. 1G and 1J show representative merged images of TUNNEL staining of brain sections of WT and M2KO (G), and brain sections of nM2KO and WT 24 h after MCAO or sham surgery (Red: NeuN; Blue: DAPI; Green: TUNNEL). FIGS. 1H and 1K show the quantification of TUNNEL positive neurons of WT and gM2KO (H), as well as nM2KO and WT sections from 5 mice/group (**, p<0.01; ANOVA, Bonferroni's t-test, mean±SEM). FIGS. 1I and 1L show the mean percentage of TUNNEL positive neurons in all NeuN positive cells (***, p<0.001; ANOVA, Bonferroni's test; mean±SEM).

FIGS. 1M-1P show the evaluation of Ca2+ overload and neuronal death caused by OGD using ratio Ca2+ imaging. Cortical neurons isolated from WT and gM2KO mice were cultured for 7 to 14 days before OGD experiments. FIG. 1M shows the ratio Ca2+ imaging showing intracellular Ca2+ changes induced by OGD in WT and gM2KO neurons. Fluorescence ratio F340/380 was used to represent intracellular Ca2+ changes (see the scale bar at the right shoulder of panel M). Neurons with increasingly elevated Ca2+ levels, such as the ones pointed with arrows, die with time, as reflected by the disappearance of the fluorescence at the next time point. Ionomycin was used to induce the maximum Ca2+ influx for normalization (not shown). FIG. 1N shows representative real-time changes of Ca2+ induced by OGD in the first 45 min. The averaged traces were from 20 neurons randomly chosen from a representative culture dish of WT and gM2KO groups (***, p<0.001, unpaired t-test, mean±SEM). FIG. 1O shows the quantification of OGD-induced Fura-2 fluorescence changes 30 min after OGD. A cohort of 238 neurons from three WT mice in 6 culture dishes and 233 neurons from gM2KO mice in 6 culture dishes were used for analysis (***, p<0.001, unpaired t-test, mean±SEM). FIG. 1P shows the OGD-induced neuronal death at 30 min, 60 min, and 90 min after OGD (***, p<0.001, unpaired t-test, mean±SEM, n=238 and 233 neurons in WT and gM2KO groups). Neuronal death was monitored as gradually reduced and eventually disappeared F340/380 fluorescence after the fluorescence reached maximal level (see representative dead cells indicated by arrows) (ns, p>0.05; ***, p<0.001; ANOVA, Bonferroni's test; mean±SEM). (see also FIGS. 10A-10D for nM2KO versus WT results).

FIGS. 1Q and 1R show the effects of Trpm2 deletion on mitochondrial function of cortical neurons during OGD evaluated by dequenching of R123 fluorescence. FIG. 1Q shows representative images of R123 labelled mitochondria before and 30 min after OGD in cultured WT and gM2KO cortical neurons. Control group (no OGD treatment) was used to show the rapid photo bleaching of R123. FIG. 1R shows the average changes of R123 fluorescence 30 min after OGD. WT neurons (n=74 for OGD, n=39 for control) and gM2KO neurons (n=123) were from 4 dishes of cultured neurons isolated from 3 mice (***, p<0.001; ANOVA, Bonferroni's test; mean±SEM).

FIGS. 1S and 1T show the enhanced TRPM2 function by OGD. TRPM2 currents elicited by a ramp protocol ranging from −100 to +100 mV in cultured cortical neurons from WT mice using pipette solutions containing 100 nM Ca2+ and 10 M ADPR (FIG. 1S). Averaged current amplitude (measured at +100 mV) was increased by 1.4 fold after OGD (FIG. 1T) (*, p<0.05, unpaired t-test, mean±SEM, n=7).

FIGS. 1U and 1V show up-regulation of TRPM2 by MCAO. TRPM2 expression level analyzed by Western blotting (FIG. 1U) was increased by 7.6 fold (FIG. 1V) in the infarcted (right) hemisphere of brains from MCAO WT mice in comparison with sham operated WT mice 24 h after surgery. Protein levels were normalized by β-Tubulin (***, p<0.001, unpaired t-test, mean±SEM, n=12/group).

FIGS. 1W-1Z show the deletion of TRPM2 abolishes the increase of surface expression of NMDARs induced by MCAO. FIG. 1W shows representative western blotting of the surface expression of GluN1a, GluN2a and GluN2b in cell membrane protein extractions of 3 brains from WT and gM2KO mice subjected to either sham surgery or MCAO. FIGS. 1X-1Z show the changes of surface expression levels of GluN1a, GluN2a and GluN2b in WT and gM2KO mice subjected to either sham surgery or MCAO. The entire hemisphere at the operation side (right hemisphere) was harvested 24 h after surgery for protein extraction. Protein levels were normalized to the membrane protein loading control pan-cadherin for quantification (ns, no statistical significance, *, p<0.05, **, p<0.01, ***, p<0.001; ANOVA, Bonferroni's test; mean±SEM, n=12).

FIGS. 2A-2L show that TRPM2 physically and functionally interacts with NMDARs.

FIGS. 2A and 2B show co-immunoprecipitation (Co-IP) of NMDARs and TRPM2 expressed in HEK-293T cells. NMDAR subunits, GluN1a, GluN2A, and GluN2B were transfected with Flag-tagged TRPM2 (Flag-TRPM2) or EGFP empty vector plasmids. FIG. 2A shows TRPM2 was immunoprecipitated (IP′d) using anti-GluN1a, anti-GluN2a and anti-GluN2b agarose, and detected using immunoblotting (WB) with anti-Flag. FIG. 2B shows cell lysates were IP′d using anti-Flag agarose and were probed using WBs with anti-GluN1a, anti-GluN2a and anti-GluN2b. All the transfections, IP, and WB were replicated for 3 times.

FIG. 2C shows the co-IP of each subunit of NMDARs and TRPM2 in HEK-293T cells in which Flag-TRPM2 was co-transfected with GluN1a (TRPM2/GluN1a), GluN2a (TRPM2/GluN2a), or GluN2b (TRPM2/GluN2b). EGFP plasmid was used as a control for transfection with each subunit of NMDARs. Cell lysates were IP′d using anti-TRPM2 agarose and were probed using WBs with anti-GluN1a, anti-GluN2a or anti-GluN2b. TRPM2 interacted with GluN2a and Glu2b, but not GluN1a. All the transfections, Co-IP and WB were repeated for 3 times.

FIGS. 2D and 2E show endogenous TRPM2 and NMDARs interaction. Co-IP of NMDARs and TRPM2 using protein extractions from brains of WT mice after MCAO. Brain extracts of gM2KO mice subjected to MCAO were used as control. FIG. 2D shows brain lysates were IP′d using anti-GluN1a, anti-GluN2a and anti-GluN2b agarose and was probed using WB with anti-TRPM2. FIG. 2E shows brain lysates were Co-IP'd using anti-TRPM2 antibody and were probed using WBs with GluN1a, GluN2a and GluN2b. All the Co-IP and WB were replicated at least using 3 mouse brains.

FIG. 2F shows representative NMDAR currents recorded by holding at −80 mV in cortical neurons cultured for 14 days from WT and gM2KO mice. NMDA at 10 UM was applied for ˜5 to 10 s. Average peak current amplitude of NMDARs from WT and gM2KO neurons is shown in inset (***, p<0.001, unpaired t-test, mean±SEM, n=20/group).

FIGS. 2G-2J show the surface expression changes of NMDARs in HEK-293T cells co-transfected with TRPM2 (NMDARs/TRPM2, or “+M2”), or EGFP plasmid as control (NMDARs/EGFP, or “Con”). FIG. 2G shows the membrane (Mem) and cytosol (Cyto) protein levels assessed with WBs. Pan-cadherin and β-tubulin were used as loading control for membrane and cytosol proteins extracts respectively. FIGS. 2H and 2J show the quantification of the expression of GluN1a, GluN2a and GluN2b in cell membrane and cytosol (*, p<0.05, **, p<0.01, unpaired t-test, mean±SEM). All transfections, extraction of membrane/cytosolic proteins and immunoblotting were repeated for at least 3 times.

FIGS. 2K and 2L show functional changes of NMDARs when co-expressed with TRPM2 in HEK293T cells. FIG. 2K shows representative NMDAR currents recorded by holding at −80 mV in HEK293T cells transfected with NMDARs/TRPM2, or NMDARs/EGFP. NMDA at 10 UM was applied for ˜5 to 10 s to activate NMDARs. FIG. 2L shows the average peak current amplitude from NMDARs/EGFP group (n=23) and NMDARs/TRPM2 group (n=27) (***, p<0.001, unpaired t-test, mean±SEM).

FIGS. 3A-3P show the identification of the interacting domain EE3 in the N-terminal of TRPM2 that mediates the physical and functional coupling between TRPM2 and NMDARs. FIG. 3F discloses SEQ ID NO: 3.

FIGS. 3A-3B show the co-IP of N-terminal (1-727) and C-terminal (1060-1503) fragments of TRPM2 (TRPM2-NT, TRPM2-CT) with NMDARs in the HEK293T cells co-transfected with NMDARs or EGFP plasmids. TRPM2-NT (FIG. 3A) and TRPM2-CT (FIG. 3B) were IP'd using anti-GluN1a, anti-GluN2a or anti-GluN2b antibodies and were probed by WBs using anti-flag (FIG. 3A) or anti-GFP (FIG. 3B). TRPM2-NT was flagged-tagged and TRPM2-CT was GFP tagged. FIG. 3A shows that TRPM2-N interacted with NMDARs as an estimated 85 kDa fragment was detected by anti-flag in NMDARs/TRPM2-N co-transfected cells but not in the NMDARs/EGFP transfected cells. FIG. 3B shows that TRPM2-CT did not interact with NMDARs as the estimated 60 kDa fragment of TRPM2-CT was only detected by anti-GFP in the input of NMDARs/TRPM2-CT co-transfected cell. These results were replicated in three independent experiments.

FIG. 3C shows the co-IP of TRPM2 with the C-terminus of GluN2a (GluN2a-CT, 1054-1068), C-terminus deleted GluN2a (GluN2a-AC, 1-1053), C-terminus of GluN2b (GluN2b-CT, 1041-1691), and C-terminus deleted GluN2b (GluN2b-AC, 1-1047) over-expressed in the HEK293T cells. All the constructs were GFP-tagged. GluN2a-CT, GluN2b-CT, GluN2a-ΔCT and GluN2b-ΔCT were IP'd using anti-TRPM2 and were probed using WB by anti-GFP. Note that only the GluN2a-CT and GluN2b-CT interacted with TRPM2. These results were replicated in three independent experiments.

FIG. 3D shows representative NMDAR currents recorded in the HEK-293T cells transfected with NMDARs/EGFP, NMDARs/TRPM2-full length (FL), NMDARs/TRPM2-NT, and NMDARs/TRPM2-CT (Left). NMDAR currents were elicited by 10 μM NMDA in tyrode solution perfused for about 7 to 10 s at −80 mV. Average current amplitude of NMDARs/EGFP group (n=9), NMDARs/TRPM2-FL group (n=8), NMDARs/TRPM2-NT group (n=8), and NMDARs/TRPM2-CT group (n=8) is shown in the bar graph at the right side (ns, p>0.05; ***, p<0.001; ANOVA, Bonferroni's test; mean±SEM).

FIG. 3E shows NMDARs-mediated Ca2+ influx in HEK-293T cells transfected with NMDARs/EGFP, NMDARs/TRPM2-FL, NMDARs/TRPM2-NT, and NMDARs/TRPM2-CT. Averaged changes of F340/380 upon NMDA perfusion at around 15 to 25 s (left), and the mean ΔF340/380 measured at ˜40 s is shown in the bar graph (right). (ns, p>0.05; **, p<0.01; ANOVA, Bonferroni's test; mean±SEM; n=17, 14, 13, and 16 respectively from 3 testing dishes/group).

FIG. 3F is a schematic diagram of the membrane topology of TRPM2. The EE3 domain is localized within the MHR4.

FIGS. 3G-3I show the identification of NMDAR binding domain at the TRPM2 N-terminus. Co-IP of Flag-TRPM2 N-terminal segments with different lengths (1-570, 1-631, 1-678 and 1-727) with NMDARs co-transfected in HEK-293T cells. FIG. 3G show that lysates were IP'd using anti-TRPM2-N (against TRPM2 N-terminus) and were probed by WBs with anti-GluN2a or anti-GluN2b. TRPM2-CT and TRPM2-FL were included as negative and positive control, respectively. FIGS. 3H-3I show that different fragments of Flag-TRPM2 were IP'd with anti-GluN2A (FIG. 3H) or GluN2b (FIG. 3I) and were probed by WBs with anti-Flag. Fragments shorter than 631 aa failed to interact with NMDARs. The results were replicated at least three times.

FIGS. 3J-3L shows the identification of EE3 domain as the binding site for TRPM2 and NMDARs. Co-IP of the TRPM2 N-terminal fragments (1-664 and 1-679) with NMDARs co-expressed in the HEK293TT cells. FIG. 3J shows that GluN2a and Glu2N2b were IP'd with N-terminal anti-TRPM2 and probed using WBs with anti-GluN2a and GluN2b. FIGS. 3K and 3L show TRPM2 N terminal segments (1-664 and 1-679) were IP'd with anti-GluN2a (FIG. 3K) and anti-GluN2b and were probed using WBs with anti-Flag. Fragment 1-664 failed to interact with NMDARs. The critical residues 665-679 for physical interaction together with additional “EE” at the position 680-681 was defined as EE3 binding domain. The results were replicated in three independent experiments.

FIGS. 3M-3P show physical and functional coupling of TRPM2 and NMDARs through EE3 domain. FIGS. 3M and 3O show that the EE3 domain deletion mutant of TRPM2 (TRPM2-ΔEE3), and EE3 mutations of TRPM2, TRPM2-QEE (E666Q and E667Q), TRPM2-EQE (E673Q and E674Q), and TRPM2-EEQ (E680Q and E681Q) were co-expressed with NMDARs in HEK293T cells for co-IP. FIG. 3M shows the TRPM2-ΔEE3 (M2-ΔEE3) was IP'd with GluN2a or GluN2b and probed with anti-Flag. TRPM2-WT (M2-WT) was used as control. FIG. 3O shows the TRPM2 mutants QEE, EQE and EEQ were IP'd with anti-TRPM2 and probed with GluN2a or GluN2b. WT-TRPM2 (EEE) was used as a control. FIG. 3N shows the NMDAR currents elicited by NMDA in HEK293T cells co-expressed with empty EGFP vector, WT-TRPM2, TRPM2-ΔΕΕ3, TRPM2-QEE, TRPM2-EQE, and TRPM2-EEQ. FIG. 3P shows the mean current amplitude (ns, p<0.05; ***, p<0.001; ANOVA, Bonferroni's test; mean±SEM, n=10/group).

FIG. 4A-4R show the N-terminal domain of TRPM2 Interacts with PKC-γ.

FIGS. 4A and 4B show the co-IP of PKCγ and TRPM2 using proteins extracted from mouse brains after MCAO (FIG. 4A), and in HEK293T cells expressing PKC□ with TRPM2-FL, TRPM2-NT, or TRPM2-CT (FIG. 4B). PKC-γ was IP'd with anti-TRPM2 and was probed by WBs using anti-PKCγ. The results were replicated in 3 independent experiments. FIG. 3B shows co-IP of PLCγ and TRPM2 in HEK293T cells transfected with PKCγ and TRPM2 (full length), PKCγ and TRPM2-N terminus (NT), or PKCγ and TRPM2-C terminus (CT). PKC-γ was co-IP'd with anti-TRPM2 and was probed by WBs using anti-PKCγ. The results were replicated in 3 independent experiments.

FIGS. 4C and 4D show the oxidative stress promotes TRPM2 and PKC□ interaction in MCAO brains. (FIG. 4C), Co-IP of TRPM2 and PKC-γ using protein extracts from WT mice brains subjected to MCAO or sham operation. PKC-γ was IP'd using anti-TRPM2 and was probed with anti-PKC-γ by WB. TRPM2 was used as control for IP protein, and β-tubulin was used as loading control. FIG. 4D shows the mean PKCγ/TRPM2 ratios in sham and MCAO mice (***, p<0.001, unpaired t-test, mean±SEM; n=6).

FIGS. 4E and 4F shows the effects of H2O2 on TRPM2 and PKCγ interaction in cultured neurons. FIG. 4E shows PKCγ was IP'd with anti-TRPM2 in protein extracts from cultured WT cortical neurons exposed to H2O2 (at 100 μM for 1 min, 3 min and 5 min, at 300 UM for 5 min and at 1 mM for 5 min), and probed with anti-PKC-γ. For IP proteins, TRPM2 was used as loading control. For lysates, β-tubulin was used as loading control. FIG. 4F shows the average PKCγ normalized to TRPM2 from 3 independent experiments. (ns, p<0.05; ***, p<0.001; ANOVA, Bonferroni's test; mean±SEM, n=3/group).

FIGS. 4G-4J show the surface expression of NMDARs in HEK-293T cells co-transfected with PKC-γ/EGFP (Con) and PKC-γ/TRPM2 (+M2) (FIG. 4G), or PKC-γ-DN/EGFP (Con) and PKC-γ-DN/TRPM2 (+M2) (FIG. 4H). Pan-cadherin was used as loading control for membrane proteins extraction, and β-tubulin was used as loading control for cytosolic proteins extraction. Membrane cytosol protein levels of NMDARs were quantified from 3 independent experiments/group (FIGS. 41-4J). (*, p<0.05, **, p<0.01, ***, p<0.001, unpaired t-test, mean±SEM).

FIGS. 4K-4N show the surface expression of NMDARs in HEK-293T cells co-transfected with TRPM2 or EGFP with the treatment of PKC activator PMA (FIG. 4K) and inhibitor Stausporine (FIG. 4L). Membrane (Mem) and cytocol (Cyto) NMDARs were analyzed and averaged from 3 independent experiments with 1 μM PMA treatment (FIG. 4N) or 1 μM Stausporine treatment (FIG. 4L) for overnight. Pan-cadherin and β-tubulin were loading controls for membrane and cytosol protein, respectively (*, p<0.05, **, p<0.01, ***, p<0.001, unpaired t-test, mean±SEM, n=3/group).

FIGS. 4O and 4P show the effects of stausporine on NMDAR currents recorded from cortical neurons cultured for 14 days. FIG. 4O show representative NMDAR currents elicited at −80 mV by 10 μM NMDA in WT and TRPM2-KO (gM2KO) neurons treated with or without Stausporine at 1 μM for overnight. FIG. 4P show the average current amplitude (ns, p>0.05; **, p<0.01; ANOVA, Bonferroni's test; mean±SEM, n=11, 10, 10, 12 neurons from 2 mice respectively).

FIGS. 4Q and 4R show the effects of Endosidin2 on NMDA currents recorded from cortical neurons cultured for 14 days.

FIG. 4S show representative NMDAR currents elicited by NMDA in WT and TRPM2-KO neurons treated with or without endosidin2 at 1 μM for overnight.

FIG. 4T shows the average current amplitude (ns, p>0.05; *, p<0.05; ANOVA, Bonferroni's test; mean±SEM, n=10 neurons/group from 2 mice respectively).

FIG. 5A-5K shows that TAT-EE3 disrupts the physical and functional interaction between TRPM2 and NMDAR.

FIGS. 5A and 5B show the effects of TAT-EE3 on interaction of TRPM2 and NMDARs. Co-IP tests of NMDARs with WT or EE3 domain mutants of TRPM2 (Flag-tagged) in co-transfected HEK293T cells. WT TRPM2 groups were treated with 10 μM TAT-SC or TAT-EE3 overnight. Lysates were IP's with anti-GluN2a (FIG. 5A) or anti-GluN2b (FIG. 5B), and probed by WBs with anti-Flag. IgG was used as control for IP. Similar to the mutants TRPM2-ΔEE3 and TRPM2-EQE, TAT-EE3 disrupted TRPM2 interaction with GluN2a and GluN2b. These results were replicated in 3 independent experiments.

FIGS. 5C-5F effects of TAT-EE3 on surface expression of NMDARs. HEK293T cells co-transfected with NMDARs with TRPM2 (+M2) or EGFP vector (Con) were treated with 10 μM TAT-SC (FIG. 5C) or TAT-EE3 (FIG. 5D) overnight. Membrane (Mem) and cytosol (Cyto) proteins of NMDARs in TAT-SC (FIG. 5E) and TAT-EE3 (FIG. 5F) treated groups were assessed by WBs and analyzed in reference to Pan-cadherin or β-tubulin (**, p<0.01, ***, p<0.001, unpaired t-test, mean±SEM). All the results were replicated in at least 3 independent experiments.

FIGS. 5G and 5H show the effects of TAT-EE3 on NMDAR currents in HEK-293T cells transfected with NMDARs and TRPM2. Transfected cells were treated with 10 μM TAT-EE3 or TAT-SC for overnight. FIG. 5G is representative NMDAR currents elicited at −80 mV by exposing to 10 μM NMDA for about 2 to 4 s. FIG. 5H shows the mean current amplitude of NMDARs/EGFP (n=13), and NMDARs/TRPM2 treated with TAT-SC (n=17) or TAT-EE3 (n=16) groups (ns, p>0.05; ***, p<0.001; ANOVA, Bonferroni's test; mean±SEM).

FIGS. 5I-5K show the effects of TAT-EE3 on PKC induced changes of NMDAR currents recorded in cortical neurons of WT and TRPM2-KO. Neurons were incubated with 10 μM TAT-SC or TAT-EE3 for overnight before current recording. FIG. 5I show representative NMDAR currents elicited by 10 μM NMDA at −80 mV before and after PMA (1 μM) perfusion for 20 s. TAT-SC or TAT-EE3 10 μM was included in the pipette solution for current recording. FIG. 5J shows the average NMDAR current amplitude before and after 1 μM PMA from WT and TRPM2-KO neurons treated with TAT-SC or TAT-EE3. FIG. 5K shows average percentage increases of NMDAR currents induced by PMA in WT and TRPM2-KO neurons treated with TAT-SC or TAT-EE3 (ns, *p>0.05; **, p<0.001; ANOVA, Bonferroni's test; mean±SEM, n=19, 14, 14, and 11, respectively).

FIGS. 6A-6E shows uncoupling TRPM2 and NMDARs protects neurons against ischemic injury in vitro.

FIGS. 6A and 6B show the effects of TAT-EE3 on NMDAR currents in WT and gM2KO cortical neurons cultured for 14 days. FIG. 6A shows a representative recording of NMDAR currents in cultured neurons treated with TAT-SC or TAT-EE3 at 10 μM for overnight before current recording. TAT-SC or TAT-EE3 10 μM was included in the pipette solution during current recording. FIG. 6B shows the average current amplitude in WT and gM2KO neurons treated with TAT-SC or TAT-EE3 (ns, p>0.05; ***, p<0.001; ANOVA, Bonferroni's test; mean±SEM, n=15, 13, 12, and 14 respectively).

FIGS. 6C-6E show the effects of TAT-EE3 on Ca2+ overload and neuronal death caused by OGD. Cultured WT and gM2KO cortical neurons were treated with 10 μM TAT-SC and TAT-EE3 for overnight before experiments. FIG. 6C shows the ratio Ca2+ imaging showing intracellular Ca2+ changes induced by OGD in TAT-EE3 or TAT-SC treated WT and gM2KO neurons. Fluorescence ratio F340/380 was used to represent intracellular Ca2+ changes (see the scale bar at the right shoulder of panel (FIG. 6C). Neurons with increasingly elevated Ca2+ levels such as the ones pointed with arrows die with time, as reflected by the disappearance of the fluorescence at the next time point. Ionomycin (Iono) was used at the end of the experiments to serve as an internal control for normalizing F340/380 (not shown). FIG. 6D shows Ca2+ increases represented by normalized F340/380 induced by OGD in the first 45 min analyzed from 20 neurons randomly chosen from each group. FIG. 6E shows the average neuronal death rate evaluated at 30, 60, and 90 min after OGD. Neuronal death was monitored as gradually reduced and eventually disappeared F340/380 fluorescence after the fluorescence reached maximal level (see representative dead cells indicated by arrows) (ns, p>0.05; ***, p<0.001; ANOVA, Bonferroni's test; mean±SEM).

FIGS. 6F-6G show the effects of TAT-EE3 on mitochondrial function evaluated using R123 de-quenching assay. Cultured cortical from gM2KO mice, nM2KO mice and WT control littermates were incubated with 10 μM TAT-SC or TAT-EE3 for overnight before OGD exposure. FIG. 6G shows R123 fluorescence changes in neurons of different groups induced by OGD. External perfusion solution aCSF containing no Ca2+ (No Ca2+) was used as a control. WT neuron without OGD exposure was used as a control (WT control) to illustrate the rapid photo bleaching of Rh123. FIG. 6F shows the mean R123 fluorescence changes induced by 30 min OGD exposure in TAT-SC (n=682), TAT-EE3 (n=499), Global M2KO (n=374) and Neuronal M2KO (549) neurons, or no-OGD Control (=258). The accumulated numbers of neurons in each group for data analysis were from 3˜5 independent experiments using neurons isolated from 3 mice/group (***, p<0.001; ANOVA, Bonferroni's test; mean±SEM).

FIGS. 7A-7K show TAT-EE3 protects mice against ischemic stroke and preserves CREB/ERK1/2 signaling in vivo.

FIGS. 7A-7C show TAT-EE3 reduces infarct size and improves neurological deficit (ND) score in WT MCAO mice. FIG. 7A shows TTC staining of brain slices 24 h after MCAO from WT mice intraperitoneally (ip) administrated with TAT-SC or TAT-EE3 at 100 nmol/kg 15 min before MCAO procedure. FIG. 7B shows the mean infarct volume after MCAO. FIG. 7C shows the average neurological deficit score. (***, p<0.001; ANOVA, Bonferroni's test; mean±SEM; n=7 for TAT-SC and n=8 for TAT-EE3 groups).

FIGS. 7D and 7E show the effects of TAT-EE3 on surface expression of NMDARs in WT and nM2KO mice 24 hrs after MCAO or sham procedure. TAT-SC and TAT-EE3 were ip administrated (100 nmol/kg) 15 mins before MCAO or sham procedure. FIG. 7D show the WB of surface expression of NMDARs from two representative samples/group. Pan-cadherin was used as loading control. FIG. 7E show the quantification of surface expression of NMDARs normalized to Pan-cadherin (ns, p>0.05; ***, p<0.001; ANOVA, Bonferroni's test; mean±SEM; n=4 mice/group).

FIGS. 7F-7H show the effects of TAT-EE3 on ERK1/2 and CREB activities in cultured neurons isolated from gM2KO and nM2KO mice, and WT littermates. WT neurons were treated with TAT-EE3 or TAT-SC for overnight before the experiments. Neurons were incubated for 1 h before experiments with 1) control (PBS), 2) NMDA (10 μM) to activate synaptic and extrasynaptic NMDARs, and 3) 4-AP 2.5 mM plus biccuculine (Bic) 50 μM (4-AP/Bic) to activate synaptic NMDARs. FIG. 7F shows the ERK1/2 and CREB activities were evaluated by detecting phosphorylated ERK1/2 (p44/42ERK1/2) and CREB (pSer133CREB). FIGS. 7G and 7H show the quantification of the active CERB and ERK1/2 in each group under three different treatment conditions (***, p<0.001; ANOVA, Bonferroni's test; mean±SEM, n=3).

FIGS. 7I-7K show the effects of TAT-EE3 and Trpm2 deletion on ERK and CREB activities in mice subjected to MCAO. FIG. 7I shows the ERK and CERB activities were evaluated by assessing the levels of pERK1/2 and pCREK in nM2KO mice and WT control littermate subjected to MCAO or sham surgery. TAT-EE3 or TAT-SC was ip administrated to WT mice 15 min before MCAO or sham surgeries. FIGS. 7J and 7K show the quantification of pERK1/2 and pCREB expressed in the brains after MCAO or sham procedure (***, p<0.001; ANOVA, Bonferroni's test; mean±SEM, n=4/group).

FIGS. 8A-8K show that TRPM2 currents recorded in WT neurons and determination of TRPM2 deletion in the global and neuron-specific knockout mice.

FIGS. 8A-8E show TRPM2 currents recorded in the cortical neurons with pipette solution containing ADPR and Ca2+. FIGS. 8A and 8C show representative currents elicited by a ramp protocol ranging from −100 to +100 mV WT neurons (FIG. 8A) but not in global TRPM2-KO (gM2KO) neurons (FIG. 8C). NMDG was used to ensure no leak contamination, and ACA (30 M) was used to inhibit TRPM2 currents. FIGS. 8B and 8D show inward and outward current measured at −100 mV and +100 mV were plotted against time (FIG. 8B). No currents were recorded in the gM2KO neuron (FIG. 8D). FIG. 8E shows the average current amplitude (at +100 mV) of TRPM2 in WT neurons and TRPM2 current was eliminated in gM2KO neurons. Please note that NMDG eliminated inward TRPM2 currents (FIGS. 8A and 8B), indicating no leak current, but meanwhile slightly reduced outward currents (in FIGS. 8A and 8B) because elimination of Ca2+ entry will gradually close TRPM2.

FIGS. 8F and 8G show the conformation of global TRPM2 knockout (gM2KO) by genotyping (FIG. 8G) and WB (FIG. 8F). FIG. 8F shows representative WB results from 3 brains of WT and gM2KO mice. FIG. 8G shows representative PCR genotyping results showing a 514 bp and 740 bp products for WT and gM2KO mice.

FIGS. 8H and 8I show representative TRPM2 currents recorded in neurons from WT and neuron-specific TRPM2-KO (nM2KO) mice. TRPM2 currents were recorded in cortical neurons from WT neurons but not in nM2KO neurons (FIG. 8H). Average currents measured at +100 mV (FIG. 8I). Please note that nM2KO eliminated TRPM2 currents.

FIGS. 8J and 8K show the conformation of neuron-specific knockout of TRPM2 by WB and genotyping. FIG. 8J shows representative WB results to detect TRPM2 deletion using cultured neurons from 3 WT and nestincre+ floxed mice (nM2KO). TRPM2 protein was largely eliminated in cultured neurons of nM2KO. The trace amount of protein detected in M2KO neuron cultures is likely from non-neuronal cells in the cultures dishes. FIG. 8K shows representative PCR results for genotyping of TRPM2 flox/flox expression. The predicted PCR products are 400 bp in TRPM2-flox/flox expressing mice (n-6) and 260 bp in a WT mouse as control.

FIGS. 9A-9B show blood flow changes measured using Laser Doppler Flowmetry (LDF). Representative data of blood flow changes measured using LDF. Blood flow was measured using LDF before and after MCAO, as well as after reperfusion. Successful MCA occlusion was confirmed by 85% reduction of cerebral blood flow OGD (***, p<0.001, unpaired t-test, mean±SEM; n=18 for WT and n=16 for gM2KO groups (FIG. 9A); and n=13 for WT and n=11 for nM2KO groups (FIG. 9B)).

FIGS. 10A-10D shows neuron-specific Trpm2 Knockout (nM2KO) protects neurons from oxygen-glucose deprivation (OGD)-induced damage.

FIG. 10A shows the evaluation of Ca2+ overload and neuronal death using Fura-2 real-time ratio Ca2+ imaging. Cortical neurons were isolated from nM2KO mice (Trpm2flox/flox, Cre+) and WT littermate control mice (Trpm2flox/flox, Cre−) and cultured for 7 to 14 days. Neurons were exposed to OGD and intracellular Ca2+ change was monitored by Fura-2 ratio Ca2+ imaging for 90 mins. Neurons with increasingly elevated Ca2+ levels, such as the ones as indicated by arrows, died and disappeared at different time points. Ionomycin was used to induce the maximum Ca2+ influx for normalization (not shown).

FIG. 10B show representative sample traces of Fura-2 real-time Ca2+ imaging normalized to ionomycin induced responses. Averaged traces from 20 neurons which were randomly chosen from WT (Trpmflox/flox, cre−) and nM2KO (Trpm2flox/flox, Cre+) groups for analysis. Ionomycin was used to induce the maximum Ca2+ influx for normalization (***, p<0.001, unpaired t-test, mean±SEM).

FIG. 10C show the quantification of OGD-induced Ca2+ changes after OGD for 30 min. 238 neurons from 3 WT mice in 6 culture dishes and 233 neurons from nM2KO mice in 6 culture dishes were used for analysis (***, p<0.001, unpaired t-test, mean±SEM).

FIG. 10D show the quantification of OGD-induced neuronal death at 30 min, 60 min, and 90 min after OGD (***, p<0.001, unpaired t-test, mean±SEM). Neuronal death was monitored as gradually reduced and eventually disappeared F340/380 fluorescence after the fluorescence reached maximal level (see representative dead cells indicated by arrows).

FIGS. 11A-11J show that TRPM2 potentiates both GluN1a/GluN2a and GluN1a/GluN2b surface expression levels.

FIGS. 11A-11C show the surface expression of GluN1a and GluN2a in HEK293T cells co-expressed with EGFP (Con) or TRPM2 (+M2). FIG. 11A shows the WB analysis of membrane and cytosol levels of GLuN1a and Glu2a. Pan-cadherin and β-tubulin were used as loading contro. FIGS. 11B and 11C show the quantification of GluN1a, GluN2a membrane (Mem) and cytosol (Cyto) expression (*, p<0.05, **, p<0.01; unpaired t-test, mean±SEM, n=3).

FIGS. 11D-11F show the surface expression of GluN1a and GluN2b in HEK293T cells co-expressed with EGFP (Con) or TRPM2 (+M2). FIG. 11D shows the WB analysis of membrane and cytosol levels of GLuN1a and Glu2b. Pan-cadherin and β-tubulin were used as loading contro. FIGS. 11E and 11F show the quantification of GluN1a, GluN2b membrane (Mem) and cytosol (Cyto) expression (*, p<0.05, **, p<0.01; unpaired t-test, mean±SEM, n=3).

FIGS. 11G and 11H show representative GluNa1/GluN2a currents (FIG. 11G) recorded in HEK-293T cells co-expressed GluN1a/GluN2a/EGFP and GluN1a/GluN2a/TRPM2, and Mean current amplitude (FIG. 11H) (***, p<0.001; ANOVA, Bonferroni's test; mean±SEM, n=11˜12).

FIGS. 11I and 11J show representative GluNa1/GluN2b currents (FIG. 11I) recorded in HEK-293T cells co-expressed GluNa/GluN2a/EGFP or GluN1a/GluN2a/TRPM2, and Mean current amplitude (FIG. 11J) (***, p<0.001; ANOVA, Bonferroni's test; mean±SEM, n=11˜12).

FIG. 12A-12H show the alignment of triple glutamate-glutamate repeats (EE3) and functional evaluation of TRPM2-EE3 domain mutants.

FIGS. 12A and 12B show the alignment of triple EE domain (EE3) in TRPM subfamily (FIG. 12A), and EE3 domain in TRPM2 of different species (FIG. 12B). FIGS. 12A and 12B disclose SEQ ID NOS 57-64, 58, and 65-70, respectively, in order of appearance.

FIGS. 12C-12G show representative TRPM2 current recordings from HEK-293T cells transfected with EE3 domain deleted TRPM2 (TRPM2-ΔEE3) and TRPM2 mutants (QEE: E666Q, E667Q; EQE: E673Q, E674Q; EEQ: E680Q, E681Q).

FIG. 12H shows the average current quantification sample traces of TRPM2 current recording from HEK-293T cells transfected with different TRPM2 mutants. 10 recordings from each group was used for analysis (ns, p>0.05; ANOVA, Bonferroni's test; mean±SEM).

FIGS. 13A and 13B show the effects of endosidin2 on surface expression levels of NMDARs.

FIG. 13A shows the surface expression of GluN1a, GluN2a and GluN2b was detected by WB in HEK-293T cells transfected with NMDARs/EGFP (Con) and NMDARs/TRPM2 (+M2) after incubation with 1 μM endosidin2 for overnight. Pan-cadherin and β-tubulin were used as loading control for membrane and cytosolic protein extraction.

FIG. 13B shows the quantification of the expression of GluN1a, GluN2a and GluN2b in cell membrane (Mem) and cytosol (Cyto) (ns, p>0.05; unpaired t-test; mean±SEM, n=3).

FIG. 14A-14C show the global TRPM2 Knockout preserves CREB and ERK-1/2 signaling after MCAO.

FIG. 14A shows western blotting analysis of changes of p-ERK 1/2, ERK-1/2, pCREB and CREB expression in the brain from WT and TRPM2-KO subjected to sham or MCAO surgery.

FIGS. 14B and 14C show the quantification of pERK-1/2 and pCREB after MCAO or sham surgery. Four mice from each group were used for quantification (**, p<0.01; ANOVA, Bonferroni's test; mean±SEM, n=4/group).

FIGS. 15A-J shows that the KKR motif in GluN2a and GluN2b is required for the direct binding to the EE3 motif in TRPM2.

FIGS. 15A and 15B show the co-immunoprecipitation (CoIP) of KKR motif deleted GluN2a (GluN2a-DKKR, FIG. 15A) and GluN2b (GluN2b-DKKR, FIG. 15B) with TRPM2. TRPM2 is FLAG-tagged and GluN2a/GluN2b is GFP tagged. IP using anti-GluN2a/b and IB with anti-Flag (upper). IP using anti-TRPM2 and IB using anti-GFP (lower).

FIG. 15C depicts the structure of GluN2a (SEQ ID NO:5) and GluN2b (SEQ ID NO: 7). The KKR motif (red label indicates the deleted sequence [DKKR (SEQ ID NO: 9)]) is localized within the C-terminal domain (CTD).

FIG. 15D depicts the schematic diagram of the in vitro binding assay. EE3 and EQE containing fragments were labeled by a N-terminal His6 tag (SEQ ID NO: 10) and KKR-containing fragments were labeled by a N-terminal GST tag.

FIGS. 15E and 15F depict the CoIP of EE3 and EQE containing fragments with KKR-containing fragments from GluN2a (FIG. 15E) and GluN2b (FIG. 15F). IP using anti-GST and IB using anti-His6.

FIGS. 15G and 15H depict the surface expression of NMDARs in HEK-293T cells cotransfected with TRPM2 and GluN2a-DKKR (FIG. 15G) and GluN2b-DKKR (FIG. 15H).

FIGS. 151 and 15J depict NMDAR current recording in HEK-293T cells cotransfected with TRPM2 and GluN2a-DKKR (FIG. 15I) and GluN2b-DKKR (FIG. 15J). **p<0.01; ***p<0.001; ANOVA, Bonferroni's test; mean±SEM.

FIG. 16A depicts the alignment of KKR domain in GluN2a and GluN2b. FIG. 16A discloses SEQ ID NOS 71-76, respectively, in order of appearance.

FIG. 16B depict the alignment of KKR domain in GluN2a in different species. FIG. 16B discloses SEQ ID NOS 77-94, respectively, in order of appearance.

FIG. 16C depict the alignment of KKR domain in GluN2b in different species. FIG. 16C discloses SEQ ID NOS 95-111, respectively, in order of appearance.

FIGS. 17A-G depict the expression and purification of EE3 containing fragment derived from TRPM2 and KKR containing fragments derived from GluN2a and GluN2b.

FIGS. 17A and 17B depict Coomassie blue stained SDS-PAGE gels showing the expression and purification process of EE3 (FIG. 17A) or EQE (FIG. 17B) containing protein fragment derived from wild-type TRPM2 or its EQE MUTANT. The band with expected molecular weight for the protein is indicated with an arrow.

FIG. 17C depicts Coomassie blue stained SDS-PAGE gels showing the expression and purification process of KKR containing protein fragment derived from GluN2a (left) or GluN2b (right). The band with expected molecular weight for the protein is indicated with an arrow.

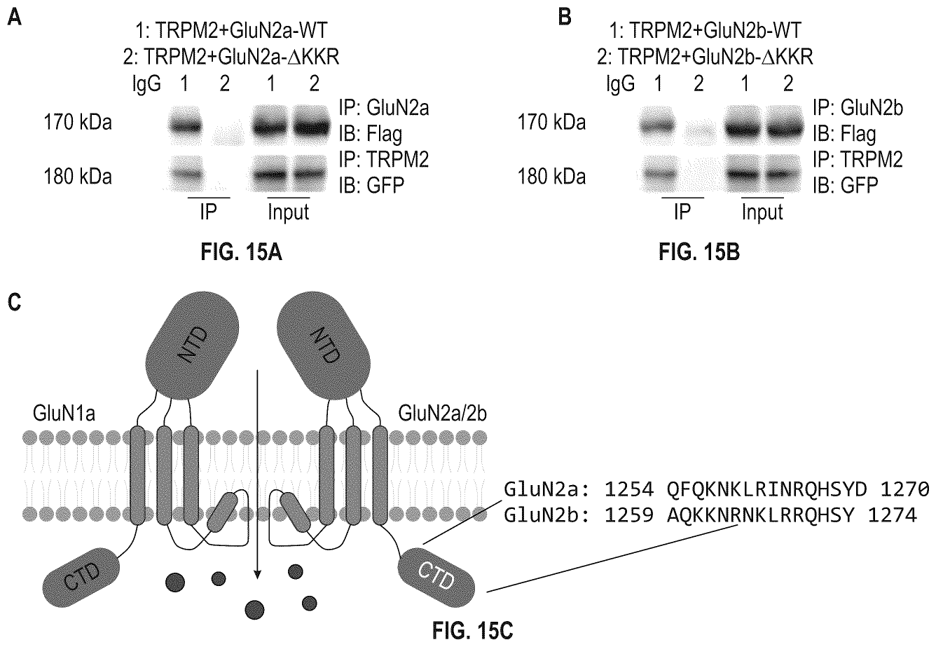

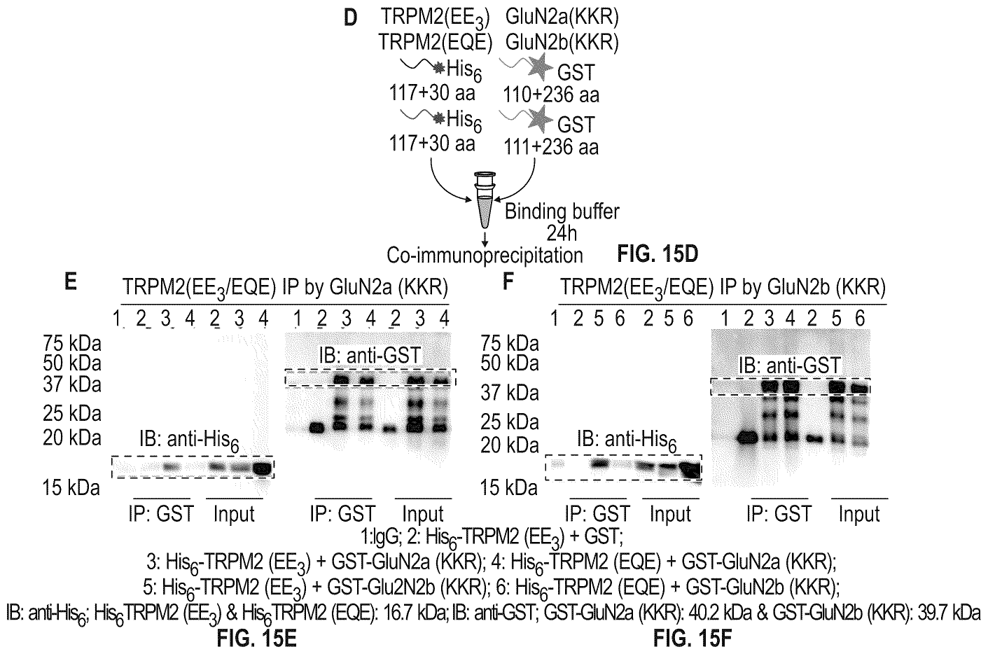

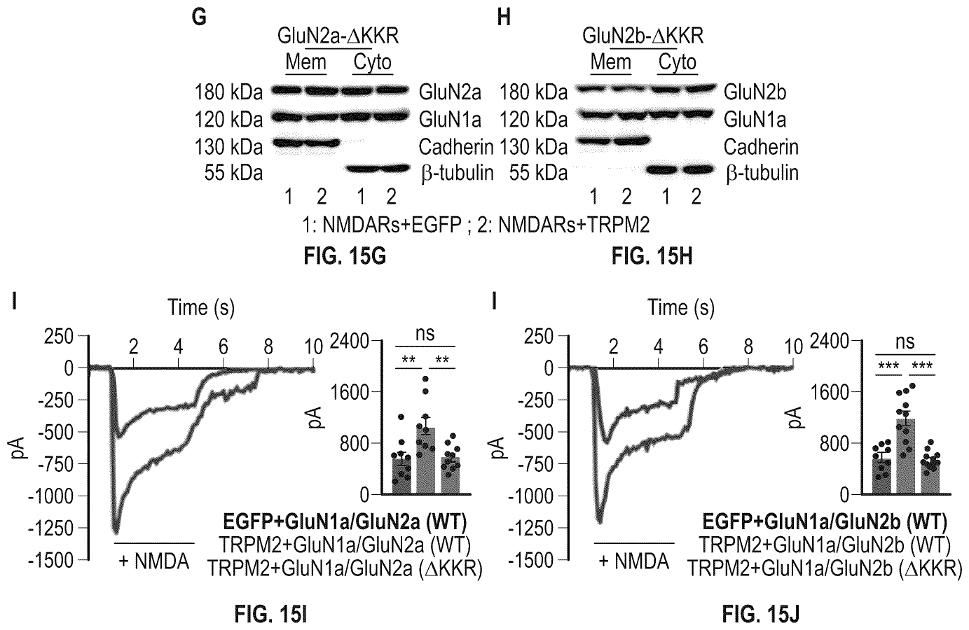

FIG. 17D depicts Coomassie blue stained SDS-PAGE gel showing the IP of the GST-tagged EE3 or EQE fragments incubated with the His6-tagged (SEQ ID NO:10) KKR fragments from either GluN2a (left) or GluN2b (right). The same samples were used in FIGS. 15E and 15F (right). Note that mutation of EEE to EQE abolished the binding.

FIG. 17E depicts Coomassie blue stained SDS-PAGE gel showing the IP of the His6-tagged (SEQ ID NO:10) KKR fragments from either GluN2a (left) or GluN2b (right) incubated with the GST-tagged EE3 or EQE fragments.

FIG. 17F and FIG. 17G depict quantification of the surface expression of GluN2a-ΔKKR and GluN2b-ΔKKR as shown in FIG. 15G and FIG. 15H, respectively (ns, p>0.05; ANOVA, Bonferroni's test; mean±SEM).

FIGS. 18A-I depict TAT-EE3 alleviates ischemic stroke by preserving prosurvival signaling.

FIG. 18A depicts CoIP of TRPM2 and GluN2a/2b in the brain lysates from WT mice after MCAO with the treatment of TAT-SC for 2 h, TAT-EQE for 2 h, and TAT-EE3 for 2, 12, and 24 h. IP using anti-GluN2a/2b and IB using anti-TRPM2.

FIG. 18B depicts the graphic illustration of injection strategy. For pre-MCAO administration, TAT-EE3 was injected intraperitoneally (i.p.) 30 min before the MCAO. For post-MCAO administration, TAT-EE3 was injected right before the reopening of the occulated MCA to best mimic the treatment of ischemic stroke under clinical situation. For examining the long-term protective effects, mice were subjected to a 1 h MCAO, and TAT-EE3 was administered every 12 h based on the pre-evaluated in vivo disrupting efficacy.

FIGS. 18C-G depic that TAT-EE3 protects WT mice against MCAO. (FIG. 18C) TTC staining of brain slices. (FIG. 18D) Mean infarct volume. (FIG. 18E) Average neurological deficit (ND) score. Average ND score (FIG. 18F) and average latency to fall time (FIG. 18G) for the long-term MCAO experiment (neuron-specific TRPM2 knockout [nM2KO]).

FIGS. 18H-I depict isolation of extrasynaptic NMDAR-mediated Ca2 response in WT and gM2KO neurons. (FIG. 18H) Representative Ca2 imaging traces (FIG. 18I) Quantification of AP burst (synaptic NMDAR-mediated response) induced by 4-AP/Bic, and extrasynaptic NMDAR-mediated response by NDMA (n=30/group). ns, no statistical significance, *p<0.05, **p<0.01, ***p<0.001; ANOVA, Bonferroni's test; mean±SEM.

### DETAILED DESCRIPTION

The inventors of the present invention have surprisingly discovered a previously unknown mechanism by which TRPM2 mediates deleterious effects leading to neuronal death during ischemic stroke. In particular, the inventors successfully demonstrated that TRPM2 exacerbates NMDARs' excitotoxicity by physically and functionally interacting with NMDARs triggered by oxidative stress. By finding the binding domains and designing a disruptive peptide TAT-EE3, the functional uncoupling of TRPM2 and NMDARs by TAT-EE3 was demonstrated to protect neurons against ischemic injury in vitro and in vivo. Thus, it is established that TRPM2 is a molecule converging the excitotoxic and non-excitotoxic pathways. Targeting TRPM2 and its coupling with NMDARs may provide a new therapeutic strategy to tackle the devastating ischemic stroke.

### 1. Definitions

Throughout the present specification and the accompanying claims the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the invention.

The terms “a,” “an,” and “the” and similar referents used in the context of describing the invention (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the invention and does not pose a limitation on the scope of the invention otherwise claimed.

Units, prefixes, and symbols are denoted in their Systeme International de Unites (SI) accepted form. Unless otherwise indicated, nucleic acids are written left to right in 5′ to 3′ orientation; amino acid sequences are written left to right in amino to carboxy orientation.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

As used herein, the term “about” refers to a range of values of plus or minus 10% of a specified value. For example, the phrase “about 200” includes plus or minus 10% of 200, or from 180 to 220, unless clearly contradicted by context.

As used herein, the term “TRPM2”, also known as “transient receptor potential melastatin 2, transient receptor potential channel 7, TRPC7, long transient receptor potential channel 2, LTRPC-2, estrogen-responsive element-associated gene 1 protein, EREG1 NUDT9L1, NUDT9H, or KNP3”, refers to a Ca2+-permeable cation channel. TRPM2 is the most abundant transient receptor potential (TRP) channel. TRPM2 is highly distributed in the central nervous system and is activated by hydrogen peroxide and agents that produce reactive oxygen/nitrogen species (ROS/RNS), increasing the Ca2+ concentration. In response to oxidative stress stimuli, TRPM2-mediated Ca2+ influx leads to cell death of a various cell types including neuron. The sequence of a human TRPM2 mRNA can be found, for example, at GenBank Accession GI: 1934153296 (NM_001320350.2; SEQ ID NO: 1). The sequence of a human TRPM2 polypeptide sequence can be found, for example, at GenBank Accession No. GI: 1934153297 (NP_001307279.2; SEQ ID NO: 2).

As used herein, the term “NMDAR”, also known as N-methyl-D-aspartate receptor, NMDA receptor, refers to an ionotropic glutamate receptor and ion channel found in neurons. Ionotropic glutamate receptors are ligand-gated ion channels that allow rapid ion influx in response to glutamate and comprise the gateway to excitotoxicity. They contains both an extracellular glutamate binding site and a transmembrane ion channel. The two main subtypes of ionotropic glutamate receptors are NMDARs and AMPA (α-amino-3-hydroxy-5-methylisoxazole-4-propionic acid) receptors (AMPARs). At the resting state, the channel pores of NMDARs are normally blocked by Mg2+. When glutamate is released from pre-synaptic sites, activated AMPARs cause a partial depolarization in the post-synaptic membrane sufficient to remove the Mg2+ block from NMDARs. Once NMDARs are activated, they flux Na+ and Ca2+ into the cell. The Ca2+ influx through NMDARs is not only critical for the normal physiological processes in neurons, but also plays a major role in initiating ischemic cell death (Choi D W. Neuron. 1988; 1 (8): 623-34). In excitotoxicity, excess glutamate release results in overactivation of NMDARs and leads to calcium overload inside the neurons. Calcium overload caused by excitotoxic mechanisms through NMDAR activation triggers a range of downstream pro-death signaling events such as calpain activation, reactive oxygen species (ROS) generation, and mitochondrial damage, resulting in cell necrosis or apoptosis (Xu J, et al., J Neurosci. 2009; 29 (29): 9330-43; Kristian T, Siesjo B K. Stroke. 1998; 29 (3): 705-18; Fujimura M, et al., J Cereb Blood Flow Metab. 1998; 18 (11): 1239-47).

NMDARs exist as multiple subtypes that differ in their molecular (subunit) composition. They are assembled as tetramers composed of two obligatory GluN1 subunits along with two GluN2 or GluN3 subunits, of which there are four (GluN2A-GluN2D) and two subtypes (GluN3A and GluN3B) respectively.

Each subunit has a typical modular architecture with two large clamshell-like extracellular domains (the N-terminal domain (NTD) involved in assembly and channel modulation and the agonist-binding domain (ABD)), a transmembrane domain (TMD) and a C-terminal domain (CTD) involved in receptor trafficking and signalling. The NTD and CTD regions are the most divergent and account for much of the functional diversity of NMDARs. Each subunit endows the receptor with distinct biophysical, pharmacological and signalling properties. The large extracellular region of the receptor harbours an array of binding sites for small-molecule ligands acting as endogenous or exogenous allosteric modulators. NMDAR subunit composition is plastic, changing during development and according to neuronal activity. Long-term synaptic plasticity of NMDARs also occurs at mature (adult) synapses and has profound consequences on cell firing and subsequent plasticity.

As used herein, the term “excitotoxicity” describes the process in which excess quantities of the excitatory neurotransmitter glutamate over-activates NMDARs and induces neuronal toxicity. This process is widely attributed to Ca2+ influx, leading to superoxide and nitric oxide production, which together generate the cytotoxic reactive oxygen species.

As used herein, an “agent” or a “modulator” refers to any compound or molecule that affects the interaction between TRPM2 and its binding protein, e.g., NMDAR. In some embodiments, the agent modulates the biological activity of TRPM2, either directly or indirectly. An agent or a modulator of TRPM2 can act directly on TRPM2, e.g., a small molecule or an antibody which binds to TRPM2 and inhibits or activates its activity and/or function. In another embodiment, the agent or the modulator of TRPM2 can act indirectly, e.g., through another molecule, e.g., a binding partner of TRPM2, e.g, NMDAR, resulting in an increase or a decrease in the activity of TRPM2. Exemplary agents suitable for use in the methods of the invention include proteins, antibodies, peptides, peptidomimetics, small molecules, nucleic acids (e.g., DNA and RNA, e.g., antisense RNAs, sdRNAs, and siRNAs), carbohydrates, lipids, and other drugs.

As used herein, the terms “modulate,” “modulation,” or “modulating” are art-recognized and refer to up-regulation (i.e., activation, stimulation, increase), or down-regulation (i.e., inhibition, suppression, reduction, or decrease) of a response, or the two in combination or apart.

As used herein, the term “inhibit” refers to a decrease in the level of interaction between TRPM2 and its binding protein, e.g., NMDAR, for example, by preventing, reducing or reversing the interaction. In some embodiments, the term “inhibit” refers to the ability to decrease, reduce, suppress, reverse, or down-regulate the activity of TRPM2. In some embodiments, the term “inhibit” refers to the ability to decrease, reduce, suppress, reverse, or down-regulate the activity of TRPM2's binding protein, e.g., NMDAR.

As used herein, the term “stimulate” refers to an increase in the level of interaction between TRPM2 and its binding protein, e.g., NMDAR. In some embodiments, the term “stimulate” refers to the ability to increase, promote, enhance, or up-regulate the activity of TRPM2. In some embodiments, the term “stimulate” refers to the ability to increase, promote, enhance, or up-regulate the activity of TRPM2's binding protein, e.g., NMDAR.

As used herein, the term “administering” means the actual physical introduction of a composition into or onto (as appropriate) a subject, a host or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “substantially decreased” and grammatical equivalents thereof refer to a level, amount, concentration of a parameter, such as a chemical compound, a metabolite, a nucleic acid, a polypeptide or a physical parameter (pH, temperature, viscosity, etc.) measured in a sample that has a decrease of at least 10%, preferably about 20%, more preferable about 40%, even more preferable about 50% and still more preferably a decrease of more than 75% when compared to the level, amount, or concentration of the same chemical compound, nucleic acid, polypeptide or physical parameter in a control sample.

As used herein, the term “substantially increased” and grammatical equivalents thereof refer to a level, amount, concentration of a parameter, such as a chemical compound, a metabolite, a nucleic acid, a polypeptide or a physical parameter (pH, temperature, viscosity, etc.) measured in a sample that has an increase of at least 30%, preferably about 50%, more preferable about 75%, and still more preferably an increase of more than 100% when compared to the level, amount, or concentration of the same chemical compound, nucleic acid, polypeptide, or physical parameter in a control sample.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

The term “subject” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject is a human, such as a human being treated or assessed for a disease, disorder or condition, a human at risk for a disease, disorder or condition, a human having a disease, disorder or condition, and/or human being treated for a disease, disorder or condition as described herein. In some embodiments, the subject does not suffer from an ongoing autoimmune disease. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included.

### II. Methods of the Invention

The present invention is based, at least in part, on the discovery that TRPM2 in neurons plays a key role in mediating the deleterious effects of TRPM2 in ischemic brain stroke. The inventors uncovered that physical and functional coupling of TRPM2 with NMDARs leads to enhanced extrasynaptic NMDAR toxicity under oxidative stress condition. Interaction of TRPM2 with PKCγ underlies the mechanism by which TRPM2 mediates enhancement of NMDAR surface expression and functional increase of NMDAR currents. In addition, the inventors identified a specific domain of TRPM2 and a specific domain of NMDAR which are responsible for interaction between TRPM2 and NMDAR, and designed a membrane permeable disrupting peptide TAT-EE3. It was demonstrated that uncoupling TRPM2 from NMDARs results in elimination of ischemic neuronal toxicity mediated by both non-excitotoxic and excitotoxic Ca2+ signaling pathways and leads to protective effects in vitro and in vivo. Specifically, administering the disrupting peptide effectively decreased the infarction size and improved the symptoms of mice undergoing middle cerebral artery occlusion, suggesting that the peptide is a promising therapeutic candidate in treating ischemic stroke in human patients.

Moreover, the newly identified NMDAR-binding domain of TRPM2 and the newly identified TRPM2-binding domain of NMDAR can be used as a drug target for developing further therapeutic candidates, such as therapeutic peptides, e.g., TAT-EE3, or small molecules, for ischemic stroke and other TRPM2-associated neurodegenerative diseases, e.g., Alzheimer diseases.

Accordingly, in one aspect, the present disclosure provides methods and compositions to treat (e.g., alleviate, ameliorate, relieve, stabilize, delay onset of, inhibit progression of, reduce severity of, and/or reduce incidence of) and/or prevent neurological injury or neurological disorders, or one or more symptoms associate with the neurological injury or neurological disorders, in a subject in need thereof.

In some embodiments, the neurological injury results from a brain injury, e.g., stroke (e.g., ischemic stroke, hemorrhagic stroke, or transient ischemic attack), traumatic brain injury (TBI), cerebral palsy, acquired brain injury, anoxic brain injury, diffuse axonal brain injury, focal brain injury, subdural hematoma, brain aneurysm, and coma.

In some embodiments, the neurological injury results from stroke, e.g., ischemic stroke. In some embodiments, the ischemic stroke is secondary to cardiac arrest.

In some embodiments, the neurological injury results from transient ischemic attack.

In some embodiments, the neurological injury results from hemorrhagic stroke.

In some embodiments, the neurological injury results from a traumatic brain injury.

Additionally, the methods and compositions may also be useful to aide in a patient's recovery from these neurological injuries, for example by improving synaptic function and memory in a patient recovering or rehabilitating following a neurological injury or during an active or prescribed rehabilitation program.

Additionally, the methods and compositions may be useful to treat and/or prevent a neurodegenerative disorder, peripheral neuropathy, or neuropathic pain, wherein the neurodegenerative disorder is selected from Alzheimer's Disease, Multiple Sclerosis, HIV-associated dementia, Huntington's Disease, Parkinson's Disease, and Amyotrophic Lateral Sclerosis. Data indicates that TRPM2 channels play a role in the development of neurodegenerative diseases, as TRPM2 channels are activated under conditions of oxidative stress and consequently contribute to injury and dysfunction. For example, Parkinson's Disease and Alzheimer's Disease are both neurodegenerative disorders in which oxidative stress has been strongly implicated, making a role for TRPM2 in the etiology of these disorders logical. Thus, this disclosure also provides methods and compositions that are useful in treating and/or preventing and/or slow down progression of neurodegenerative disorders, such as, Parkinson's Disease and Alzheimer's Disease.

In some embodiments, the neurodegenerative disease is Alzheimer's disease. In some such embodiments, the methods comprise reducing beta-amyloid toxicity in a neuron in the subject.

In some embodiments, the neurodegenerative disease is Parkinson's disease. In some such embodiments, the methods comprise reducing apoptosis of dopaminergic neurons in the subject.

Additionally, the methods and compositions may be useful to enhance cognitive function in a subject. For example, the methods and compositions may be administered to a subject to enhance synaptic function and/or enhance memory. These effects may reduce or slow the progress of a neurodegenerative disorder or enhance recovery from a neurological injury.

Additionally, the methods and compositions may be useful to treat and/or prevent inflammation, ischemia, atherosclerosis, asthma, autoimmune disease, diabetes, arthritis, allergies, transplant rejection, infection, pain from diabetic neuropathy, gastric pain, postherpetic neuralgia, fibromyalgia, surgery, or chronic back pain.

In these methods, the subject may be human. The subject may be male or female.

These treatment methods comprise administering to a subject an agent or a composition comprising the agent that inhibits the activity of TRPM2. Agents suitable for use in the methods of the present invention includes any compound or molecule that can inhibit the biological activity of TRPM2, for example, by inhibiting the interaction between TRPM2 and NMDAR.

An agent can modulate the activity of TRPM2 either directly or indirectly. In some embodiment, the agent is an inhibitory agent. In some embodiments, the agent can act directly on TRPM2, e.g., an antagonist antibody which binds to TRPM2 and inhibits its activity and/or function. Alternatively, an inhibitory agent of TRPM2 can act indirectly on TRPM2 (e.g., through another molecule, e.g., a binding partner of TRPM2, e.g., NMDAR) resulting in a decreased activity. Binding partners of TRPM2 can be identified by any methods known in the art. For example, protein-protein interaction between TRPM2 and binding partners can be identified by co-immunoprecipitation in which the binding of a pair of proteins of interest is determined by forming a co-precipitate with an antibody in vitro. Alternatively, the yeast two-hybrid or phage display approach may be employed to screen for binding partners of TRPM2. In some embodiments, chemical cross-linking assays followed by mass spectrometry analysis can be used to identify interacting proteins.

In some embodiments, the agent inhibits the interaction between TRPM2 and NMDAR by targeting the binding site of NMDAR, e.g., a NMDAR-binding site, on TRPM2. An agent may target the NMDAR-binding site by binding to the NMDAR-binding site, thus blocking interaction between TRPM2 and NMDAR. Alternatively, an agent may target the NMDAR-binding site by mimicing or resembling the amino acid sequence of NMDAR-binding site, thus competing with NMDAR for TRPM2 binding.

In some embodiments, the agent inhibits the interaction between TRPM2 and NMDAR by targeting the binding site of TRPM2, e.g., a TRPM2-binding site, on NMDAR. An agent may target the TRPM2-binding site by binding to the TRPM2-binding site, thus blocking interaction between TRPM2 and NMDAR. Alternatively, an agent may target the TRPM2-binding site by mimicking or resembling the amino acid sequence of TRPM2-binding site, thus competing with TRPM2 for NMDAR binding.

The inventors of the present invention have successfully identified a specific domain of TRPM2 which is responsible for interaction between TRPM2 and NMDAR. The NMDAR-binding site is located in the N-terminus of TRPM2. In some embodiments, the NMDAR-binding site comprises amino acid residues 631-679 of TRPM2. In some embodiments, the NMDAR-binding site comprises the N-terminal amino acid residues 665-681 of TRPM2, i.e., the amino acid sequence of EEEDTDSSEEMLALAEE (SEQ ID NO:3; also referred herein as “TRPM2-EE3”). As shown in the working examples, when the TRPM2-EE3 domain was deleted from full length TRPM2, interaction between TRPM2 and NMDARs was completely disrupted.

The inventors of the present invention have also successfully identified a specific domain of NMDAR which is responsible for interaction between TRPM2 and NMDAR. The TRPM2-binding site is located in the C-terminus of NMDAR. In some embodiments, the TRPM2-binding site comprises amino acid residues 1254-1270 of GluN2a subunit of NMDAR, i.e., the amino acid sequence of QFQKNKLRINRQHSYD (SEQ ID NO:5), or residues 1256-1268 of GluN2a subunit of NMDAR, i.e., the amino acid sequence of QKNKLRINRQHS (SEQ ID NO:6). In some embodiments, the TRPM2-binding site comprises amino acid residues 1254-1274 of GluN2b subunit of NMDAR, i.e., the amino acid sequence of AQKKNRNKLRRQHSY (SEQ ID NO:7), or residues 1261-1272 of GluN2b subunit of NMDAR, i.e., the amino acid sequence of KKNRNKLRRQH (SEQ ID NO: 8). As shown in the working examples, deletion of these sequences from NMDAR abolished the interaction between TRPM2 and NMDAR.

Exemplary agents suitable for use in the methods of the invention include small molecule, peptides, antagonist antibodies, or antigen-binding fragment thereof, recombinant fusion proteins, or interfering nucleic acid molecules (e.g., antisense RNAs, sdRNAs, and siRNAs), etc.

In some embodiments, the agent comprises a small molecule that binds to the NMDAR-binding site. In some embodiments, the agent comprises an antagonist anti-TRPM2 antibody that binds to the NMDAR-binding site.

In some embodiments, the agent comprises a peptide comprising an amino acid sequence with at least 50%, e.g., 55%, 60%, 65%, 70%, 75%, 80%, 85%, 90%, 91%, 92%, 93%, 94%, 95%, 96%, 97%, 98%, or 99% sequence identity to the amino acid sequence of YGRKKRRQRRREEDTDSSEEMLALAEE (SEQ ID NO:4; also refered herein as “TAT-EE3”). In some embodiments, the peptide comprises an amino acid sequence differing by 1, 2, 3, 4, or 5 residues from the amino acid sequence of YGRKKRRQRRREEDTDSSEEMLALAEE (SEQ ID NO:4). In some embodiments, the peptide comprises the amino acid sequence of

(SEQ ID NO: 4)
YGRKKRRQRRREEDTDSSEEMLALAEE.

In some embodiments, the agent comprises a peptide comprising an amino acid sequence with at least 50%, e.g., 55%, 60%, 65%, 70%, 75%, 80%, 85%, 90%, 91%, 92%, 93%, 94%, 95%, 96%, 97%, 98%, or 99% sequence identity to the amino acid sequence of EEEDTDSSEEMLALAEE (SEQ ID NO:3). In some embodiments, the agent comprises a peptide comprising an amino acid sequence differing by 1, 2, 3, 4, or 5 residues from the amino acid sequence of EEEDTDSSEEMLALAEE (SEQ ID NO:3). In some embodiments, the peptide does not comprise the amino acid sequence of

(SEQ ID NO: 3)
EEEDTDSSEEMLALAEE.

In some embodiments, the agent comprises a small molecule that binds to the TRPM2-binding site. In some embodiments, the agent comprises an antagonist anti-NMDAR antibody that binds to the TRPM2-binding site.

In some embodiments, the agent comprises a peptide comprising an amino acid sequence with at least 50%, e.g., 55%, 60%, 65%, 70%, 75%, 80%, 85%, 90%, 91%, 92%, 93%, 94%, 95%, 96%, 97%, 98%, or 99% sequence identity to the amino acid sequence of QFQKNKLRINRQHSYD (SEQ ID NO:5) or the amino acid sequence of QKNKLRINRQHS (SEQ ID NO:6). In some embodiments, the peptide comprises an amino acid sequence differing by 1, 2, 3, 4, or 5 residues from the amino acid sequence of QFQKNKLRINRQHSYD (SEQ ID NO:5), or the amino acid sequence of QKNKLRINRQHS (SEQ ID NO:6). In some embodiments, the peptide does not comprise the amino acid sequence of QFQKNKLRINRQHSYD (SEQ ID NO:5), or the amino acid sequence of QKNKLRINRQHS (SEQ ID NO:6).

In some embodiments, the agent comprises a peptide comprising an amino acid sequence with at least 50%, e.g., 55%, 60%, 65%, 70%, 75%, 80%, 85%, 90%, 91%, 92%, 93%, 94%, 95%, 96%, 97%, 98%, or 99% sequence identity to the amino acid sequence of AQKKNRNKLRRQHSY (SEQ ID NO:7), or the amino acid sequence of KKNRNKLRRQH (SEQ ID NO:8). In some embodiments, the peptide comprises an amino acid sequence differing by 1, 2, 3, 4, or 5 residues from the amino acid sequence of AQKKNRNKLRRQHSY (SEQ ID NO:7), or the amino acid sequence of KKNRNKLRRQH (SEQ ID NO:8). In some embodiments, the peptide does not comprise the amino acid sequence of AQKKNRNKLRRQHSY (SEQ ID NO:7), or the amino acid sequence of KKNRNKLRRQH (SEQ ID NO:8).

In certain embodiments, administrating to the subject the agent or a composition comprising the agent inhibits or reverses the interaction between TRPM2 and NMDAR. In certain embodiments, administering to the subject the agent or the composition comprising the agent inhibits the non-excitotoxic calcium signaling pathway mediated by TRPM2. In certain embodiments, administering to the subject the agent or the composition comprising the agent inhibits the excitotoxic calcium signaling pathway mediated by NMDAR. In certain embodiments, administering to the subject the agent or the composition comprising the agent decreases the NMDAR surface expression in neurons. In certain embodiments, administering to the subject the agent or the composition comprising the agent decreases the NMDAR current in neurons. In certain embodiments, administering to the subject the agent or the composition comprising the agent reduces mitochondrial membrane depolarization induced by ischemic injury in neurons. In certain embodiments, administering to the subject the agent or the composition comprising the agent prevents neuronal death induced by ischemic injury. In certain embodiments, administering to the subject the agent or the composition comprising the agent reduces infarct volume and/or improves neurological behavior score.

The methods further comprise administering to the subject an additional therapeutic agent or therapy currently known or later discovered to be effective in the prevention and/or treatment of neurological injury or neurological disorder, e.g., stroke, or neurological damage following stroke. The additional therapeutic agent may be an anticoagulant or clot-dissolving medicine, such as aspirin, clopidogrel or tissue plasminogen activator (tPA). The additional therapeutic agent may be an ACE Inhibitor, such as Lisinopril, or a blood thinner, such as warfarin, or heparin, or apixaban, or a statin, such as atorvastatin or rosuvastatin, or irbesartan, or reteplase, or alteplase.

Contemplated therapies include surgery, such as carotid endarterectomy, or angioplasty, or stent placement. Contemplated therapies may also include physical or mental rehabilitation programs, which have proven particularly efficacious for rehabilitation and recovery following stroke and traumatic brain injury.

The agents or compositions comprising the agents of this disclosure may be administered prior to, concurrently with, or after the administration of the additional therapeutic agent and/or therapy. These methods may include a step of assessing the efficacy of the therapeutic treatment. Such assessment of efficacy may be based on any number of assessment results. Depending on the level of efficacy assessed, the dosage of the neuroprotective peptides of this disclosure may be adjusted up or down, as needed.

Thus, by “in combination with,” it is not intended to imply that the agents or compositions of this disclosure and additional agent or therapy must be administered at the same time or formulated for delivery together, although these methods of delivery are within the scope of this disclosure. Furthermore, it will be appreciated that therapeutically active agents utilized in combination may be administered together in a single composition or administered separately in different compositions. In general, each agent will be administered at a dose and/or on a time schedule determined for that agent.

In general, each agent will be administered at a dose and on a time schedule determined for that agent. Additionally, this disclosure encompasses the delivery of the compositions in combination with agents that may improve their bioavailability, reduce or modify their metabolism, inhibit their excretion, or modify their distribution within the body.

The particular combination of therapies (e.g., therapeutics or procedures) to employ in a combination regimen will take into account compatibility of the desired therapeutics and/or procedures and the desired therapeutic effect to be achieved. In general, it is expected that agents utilized in combination will be utilized at levels that do not exceed the levels at which they are utilized individually. In some embodiments, the levels utilized in combination will be lower than those utilized individually.

### III. Agents for Use in the Methods of the Invention

The present invention also provides agents that can modulate, e.g., inhibit, the activity of TRPM2, e.g., by inhibiting the interaction between TRPM2 and a binding partner, e.g., NMDAR. Specifically, the inventors identified a specific domain of TRPM2 and a specific domain of NMDAR which are responsible for interaction between TRPM2 and NMDAR. The NMDAR-binding domain of TRPM2 and the TRPM2-binding domain of NMDAR can be used as a drug target for developing further therapeutic candidates, such as therapeutic peptides, e.g., TAT-EE3, or small molecules, for neurological injury or neurological disorders, e.g., ischemic stroke and other TRPM2-associated neurodegenerative diseases, e.g., Alzheimer diseases.

Accordingly, molecules which modulate, e.g., inhibit, the activity of TRPM2, and/or molecules which modulate, e.g., inhibit, the activity of TRPM2's binding partner, e.g., NMDAR, are useful in the methods of the present invention. Exemplary agents can modulate, e.g., inhibit, the association between TRPM2 and NMDAR.

### Small Molecule Inhibitors

An inhibitory agent for use in the methods of the present invention can be a small molecule. Small molecules are chemical compounds that inhibit the activity of TRPM2, e.g., by inhibiting the interaction between TRPM2 and NMDAR. Such compounds can be either natural products or members of a combinatorial chemistry library, and can be identified using screening assays, as described in detail below.

In some embodiments, an inhibitory agent is a small molecule, e.g., a small molecule inhibitor for TRPM2, or a small molecule inhibitor for binding proteins of TRPM2, e.g., NMDAR.

The small molecule inhibitors of the present invention may block the interaction between TRPM2 and NMDAR. For example, the small molecule inhibitors bind to the NMDAR binding domain of TRPM2. In other embodiments, the small molecule inhibitors of the present invention may bind to the region in NMDAR that interacts with TRPM2, thereby blocking the interaction between TRPM2 and NMDAR.

In some embodiments, the small molecule inhibitors are selected to bind domains sharing homology to an NMDAR-binding domain of the TRPM2. For example, a small molecule of the present invention may be directed toward a domain which is at least 50% identical, at least 60% identical, at least 70% identical, at least 80% identical, at least 90% identical, or at least 95% or 99% identical to the NMDAR-binding domain of the TRPM2. Such a small molecule would be capable of binding to the NMDAR-binding domain of the TRPM2, thus blocking the interaction between TRPM2 and NMDAR.

In some embodiments, the small molecule binds to specific sequences of the TRPM2 protein, e.g., a NMDAR-binding site, e.g., amino acid residues 631-679 of TRPM2 protein, or amino acid residues 665-681 of TRPM2 protein. In some embodiments, the NMDAR-binding site comprises an amino acid sequence of

(SEQ ID NO: 3)
EEEDTDSSEEMLALAEE (“TRPM2-EE3”).

In some embodiments, the small molecule inhibitors are selected to bind domains sharing homology to a TRPM2-binding domain of the NMDAR. For example, a small molecule of the present invention may be directed toward a domain which is at least 50% identical, at least 60% identical, at least 70% identical, at least 80% identical, at least 90% identical, or at least 95% or at least 99% identical to the TRPM2-binding domain of the NMDAR. Such a small molecule would be capable of binding to the TRPM2-binding domain of the NMDAR, thus blocking the interaction between TRPM2 and NMDAR.

In some embodiments, the small molecule binds to specific sequences of the NMDAR protein, e.g., a TRPM2-binding site. The TRPM2-binding site is located in the C-terminus of NMDAR. In some embodiments, the TRPM2-binding site comprises amino acid residues 1254-1270 of GluN2a subunit of NMDAR, i.e., the amino acid sequence of QFQKNKLRINRQHSYD (SEQ ID NO:5), or residues 1256-1268 of GluN2a subunit of NMDAR, i.e., the amino acid sequence of QKNKLRINRQHS (SEQ ID NO: 6). In some embodiments, the TRPM2-binding site comprises amino acid residues 1254-1274 of GluN2b subunit of NMDAR, i.e., the amino acid sequence of AQKKNRNKLRRQHSY (SEQ ID NO:7), or residues 1261-1272 of GluN2b subunit of NMDAR, i.e., the amino acid sequence of KKNRNKLRRQH (SEQ ID NO:8).

### Mutant Proteins

Variants of TRPM2 protein or variants of TRPM2's binding proteins (e.g., NMDAR) that function as antagonists and inhibit the interaction TRPM2 and NMDAR can also be used in the methods of the present invention.

In some embodiments, the agent is a mutant TRPM2 protein with a deletion of the NMDAR-binding site and has a reduced binding activity for NMDAR. For example, a mutant TRPM2 protein with a deletion of amino acid residues 631-679, or residues 665-681 can be used as an inhibitory agent in the present invention. In some embodiments, the agent is a mutant TRPM2 protein with a substitution mutation at residue 674, a substitution mutation at residue 675, or substitution mutations at both residues 674 and 675 of TRPM2 protein, which correspond to the middle “EE” of the TRPM2-EE3 domain. As shown in Example 1, deletion of the TRPM2-EE3 domain from the full-length TRPM2 resulted in a complete disruption of the interaction between TRPM2 and NMDAR. In addition, when the middle “EE” of the TRPM2-EE3 domain was replaced by “QQ” (glutamine), the TRPM2-EQE mutant failed to interact with NMDARs, indicating that the EE residues in the middle of the TRPM2-EE3 domain are also critical for the TRPM2-NMDAR interaction.

In some embodiments, variants of TRPM2's binding proteins (e.g., NMDAR) block the interaction between TRPM2 and NMDAR. In some embodiments, a mutant NMDAR which loses the ability to interact with TRPM2 can function as an inhibitory agent in the present invention, for example, a NMDAR protein without a C-terminal domain. In some embodiments, the agent is a mutant NMDAR protein with mutations, e.g., substitutions or deletions, at residues 1254-1270 of GluN2a subunit of NMDAR, or residues 1256-1268 of GluN2a subunit of NMDAR. In some embodiments, the agent is a mutant NMDAR protein with mutations, e.g., substitutions or deletions, at residues 1254-1274 of GluN2b subunit of NMDAR, or residues 1261-1272 of GluN2b subunit of NMDAR.

A recombinant mutant protein for TRPM2 and NMDAR for use in the methods of the present invention may be generated from a recombinant vector according to methods know in the art. The recombinant vectors can comprise a nucleic acid encoding a mutant TRPM2 or a mutant NMDAR protein in a form suitable for expression of the nucleic acid in a host cell.

In some embodiments, the recombinant vectors may include one or more regulatory sequences, selected on the basis of the host cells to be used for expression, which is operably linked to the nucleic acid sequence to be expressed (i.e., a recombinant expression vector). Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, CA (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including mutant proteins or peptides, encoded by nucleic acids as described herein.

The recombinant expression vectors of the invention can be designed for expression of a polypeptide, or functional fragment thereof, in prokaryotic (e.g., E. coli) or eukaryotic cells (e.g., insect cells using baculovirus expression vectors, yeast cells or mammalian cells). Suitable host cells may include, but not limited to E. coli cells, Bacillus cells, Saccharomyces cells, Pochia cells, NS0 cells, COS cells, Chinese hamster ovary (CHO) cells, myeloma cells, or cells as described herein.

Another aspect of the invention pertains to host cells into which a recombinant vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation.

### Antagonist Antibodies or Antigen-Binding Portion Thereof

The invention further contemplates methods and compositions comprising an antagonist antibody, or antigen binding portion thereof, which inhibits the activity of TRPM2 and/or its binding protein, e.g., NMDAR.

In some embodiments, an antagonist antibody of TRPM2 and/or an antagonist antibody of TRPM2's binding protein, e.g., NMDAR, or an antigen binding portion thereof, block the interaction between TRPM2 and NMDAR.

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. An “antibody” refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from N terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., TRPM2, or NMDAR). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region; (iv) a Fd fragment consisting of the VH and CH1 domains; (v) a Fv fragment consisting of the VL and VH domains of a single arm of an antibody, (vi) a dAb fragment (Ward et al. (1989) Nature 341:544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An “isolated antibody”, as used herein, is intended to refer to an antibody that is substantially free of other antibodies having different antigenic specificities (e.g., an isolated antibody that specifically binds to, e.g., TRPM2 or NMDAR, is substantially free of antibodies that specifically bind antigens other than TRPM2 or NMDAR). Moreover, an isolated antibody may be substantially free of other cellular material and/or chemicals. An “isolated antibody” may, however, include polyclonal antibodies, which all bind specifically to, e.g., TRPM2 or NMDAR.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies are produced by a hybridoma, which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences. It will be appreciated by one of skill in the art that when a sequence is “derived” from a particular species, said sequence may be a protein sequence, such as when variable region amino acids are taken from a murine antibody, or said sequence may be a DNA sequence, such as when variable region encoding nucleic acids are taken from murine DNA. A humanized antibody may also be designed based on the known sequences of human and non-human (e.g., murine or rabbit) antibodies. The designed antibodies, potentially incorporating both human and non-human residues, may be chemically synthesized. The sequences may also be synthesized at the DNA level and expressed in vitro or in vivo to generate the humanized antibodies.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” or “antibody mimic” is intended to refer to molecules capable of mimicking an antibody's ability to bind an antigen, but which are not limited to native antibody structures. Examples of such antibody mimetics include, but are not limited to, Adnectins (i.e., fibronectin based binding molecules), Affibodies, DARPins, Anticalins, Avimers, and Versabodies all of which employ binding structures that, while they mimic traditional antibody binding, are generated from and function via distinct mechanisms. The embodiments of the instant invention, as they are directed to antibodies, or antigen-binding portions thereof, also apply to the antibody mimetics described above.

Standard assays to evaluate the binding ability of the antibodies toward TRPM2 are known in the art, including for example, ELISAs, Western blots and RIAs. The binding kinetics (e.g., binding affinity) of the antibodies also can be assessed by standard assays known in the art, such as by ELISA, Scatchard and Biacore analysis.

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

Polyclonal antibodies of the present invention can be produced by a variety of techniques that are well known in the art. Polyclonal antibodies are derived from different B-cell lines and thus may recognize multiple epitopes on the same antigen. Polyclonal antibodies are typically produced by immunization of a suitable mammal with the antigen of interest, e.g., TRPM2. Animals often used for production of polyclonal antibodies are chickens, goats, guinea pigs, hamsters, horses, mice, rats, sheep, and, most commonly, rabbit. Standard methods to produce polyclonal antibodies are widely known in the art and can be combined with the methods of the present invention (e.g., U.S. Pat. Nos. 4,719,290, 6,335,163, 5,789,208, 2,520,076, 2,543,215, and 3,597,409, the entire contents of which are incorporated herein by reference.

Monoclonal antibodies of the present invention can be produced by any of a variety of techniques known to those of ordinary skill in the art (see, for example, Harlow and Lane, Antibodies: A Laboratory Manual, Cold Spring Harbor Laboratory, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein (Eur. J. Immunol. 6:511-519, 1976), and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding antibodies employed in the disclosed methods may be isolated and sequenced using conventional procedures. Recombinant antibodies, antibody fragments, and/or fusions thereof, can be expressed in vitro or in prokaryotic cells (e.g. bacteria) or eukaryotic cells (e.g. yeast, insect or mammalian cells) and further purified as necessary using well known methods.

More particularly, monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals. Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

The selected hybridomas would then be serially diluted and cloned into individual antibody-producing cell lines, which clones may then be propagated indefinitely to provide MAbs. The cell lines may be exploited for MAb production in two basic ways. A sample of the hybridoma may be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, may then be tapped to provide MAbs in high concentration. The individual cell lines also may be cultured in vitro, where the MAbs are naturally secreted into the culture medium from which they may be readily obtained in high concentrations. MAbs produced by either means may be further purified, if desired, using filtration, centrifugation and various chromatographic methods such as HPLC or affinity chromatography.

Large amounts of the monoclonal antibodies of the present invention also may be obtained by multiplying hybridoma cells in vivo. Cell clones are injected into mammals which are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

In accordance with the present invention, fragments of the monoclonal antibody of the invention may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064).

Digestion of antibodies to produce antigen-binding fragments thereof can be performed using techniques well known in the art. For example, the proteolytic enzyme papain preferentially cleaves IgG molecules to yield several fragments, two of which (the “F (ab)” fragments) each comprise a covalent heterodimer that includes an intact antigen-binding site. The enzyme pepsin is able to cleave IgG molecules to provide several fragments, including the “F(ab′).sub.2” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent V.sub.H::V.sub.L heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

Antibody fragments that specifically bind to the protein biomarkers disclosed herein can also be isolated from a library of scFvs using known techniques, such as those described in U.S. Pat. No. 5,885,793.

A wide variety of expression systems are available in the art for the production of antibody fragments, including Fab fragments, scFv, VL and VHs. For example, expression systems of both prokaryotic and eukaryotic origin may be used for the large-scale production of antibody fragments. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins.

Following screening and sequencing, antibodies may be produced using recombinant methods and compositions, e.g., as described in U.S. Pat. No. 4,816,567, incorporated by reference herein. An isolated nucleic acid encoding, for example, an anti-TRMP2 antibody is used to transform host cells for expression. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell).

For recombinant production of an anti-TRPM2 antibody, a nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W 138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR.sup.-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### TPMR2-Derived or NMDAR-Derived Peptides

An inhibitory agent for use in the methods of the present invention is a peptidic compound derived from the amino acid sequence of TRPM2 (e.g., the sequence disclosed herein as SEQ ID NO: 2) and/or its binding proteins, e.g., NMDAR. In particular, the inhibitory compound comprises a portion of TRPM2 or NMDAR (or a mimetic thereof) that mediates interaction of TRPM2 with NMDAR, such that contact of TRPM2 or NMDAR with this peptidic compound competitively inhibits the interaction of TRPM2 and NMDAR. For example, a peptide derived from TRPM2 that comprises the amino acid sequence in the NMDAR-binding domain may serve as an inhibitory modulator. Alternatively, the peptide resembles a fragment of the NMDAR-binding site of the TRPM2 protein, and can act to specifically block the interaction between TRPM2 and NMDAR. Similarly, a peptide derived from NMDAR that comprises the amino acid sequence in the TRPM2-binding domain may serve as an inhibitory modulator. A peptide that resembles a fragment of the TRPM2-binding site of the NMDAR protein can also act to specifically block the interaction between TRPM2 and NMDAR.

The inventors identified a specific domain of TRPM2 and a specific domain of NMDAR which are responsible for interaction between TRPM2 and NMDAR, and designed a membrane permeable disrupting peptide, i.e., TAT-EE3. It was demonstrated that uncoupling TRPM2 from NMDARs eliminates ischemic neuronal toxicity mediated by both non-excitotoxic and excitotoxic Ca2+ signaling pathways and leads to protective effects in vitro and in vivo. Specifically, administering the disrupting peptide effectively decreased the infarction size and improved the symptoms of mice undergoing middle cerebral artery occlusion, a surgery mimics ischemic stroke in humans, suggesting that the peptide is a promising therapeutic in treating ischemic stroke in human patients.

Accordingly, in one aspect, the present invention provides a cell permeable peptide TAT-EE3, having the sequence YGRKKRRQRRREEDTDSSEEMLALAEE (SEQ ID NO:4), or a multimer, derivative, or variant thereof.

The TAT-EE3 peptide inhibits interaction between TRPM2 and NMDAR, and functionally uncouples TRPM2 and NMDARs. In some embodiments, the TAT-EE3 peptide effectively eliminated the increase of NMDAR surface expression and functional increase of NMDAR currents. In some embodiments, the TAT-EE3 peptide reduces or eliminates ischemic injury-induced excitotoxicity in vitro, and significantly attenuated ischemic injury-induced neuronal death in vivo. In some embodiments, TAT-EE3 protects neurons against ischemic injury.

In some embodiments, disclosed herein are peptides that are at least 50% identical (e.g., have at least 60%, 70%, 80%, 90%, 95% or more sequence identity) to TAT-EE3 and that retain at least one neuroprotective property thereof. Preferably, a peptide variant of the neuroprotective peptides of this disclosure will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to TAT-EE3. Alternatively, the variant peptide(s) will have no more than 1, 2, 3, 4, 5, 6, 7, 8, 9, 10, 11, 12, 13, 14, 15, 16, 17, 18, 19, 20, 21, 22, 23, 24, 25, or 26 amino acid substitutions as compared to TAT-EE3.

In some embodiments, the peptide comprises a dimer, a trimer, a tetramer, or a multimer of TAT-EE3.

In another aspect, the present invention provides a cell permeable peptide having the sequence of EEEDTDSSEEMLALAEE (SEQ ID NO:3), or a multimer, derivative, or variant thereof.

In some embodiments, disclosed herein are peptides that are at least 50% identical (e.g., have at least 60%, 70%, 80%, 90%, 95% or more sequence identity) to the amino acid sequence of EEEDTDSSEEMLALAEE (SEQ ID NO:3) and that retain at least one neuroprotective property thereof. Preferably, a peptide variant of the neuroprotective peptides of this disclosure will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to the amino acid sequence of EEEDTDSSEEMLALAEE (SEQ ID NO:3).

In some embodiments, the peptide does not comprise an amino acid sequence that is 100% identical to the amino acid sequence of SEQ ID NO:3. In some embodiments, the variant peptide(s) will have no more than 1, 2, 3, 4, 5, 6, 7, 8, 9, 10, 11, 12, 13, 14, 15, 16, 17, 18, 19, 20, 21, 22, 23, 24, 25, or 26 amino acid substitutions as compared to the amino acid sequence of EEEDTDSSEEMLALAEE (SEQ ID NO:3).

In some embodiments, the peptide comprises a dimer, a trimer, a tetramer, or a multimer of the amino acid sequence of EEEDTDSSEEMLALAEE (SEQ ID NO:3).

In another aspect, the present invention provides a cell permeable peptide having the sequence of QFQKNKLRINRQHSYD (SEQ ID NO:5), or the amino acid sequence of QKNKLRINRQHS (SEQ ID NO:6), or a multimer, derivative, or variant thereof.

In some embodiments, disclosed herein are peptides that are at least 50% identical (e.g., have at least 60%, 70%, 80%, 90%, 95% or more sequence identity) to the amino acid sequence of QFQKNKLRINRQHSYD (SEQ ID NO:5) or the amino acid sequence of QKNKLRINRQHS (SEQ ID NO:6), and that retain at least one neuroprotective property thereof. Preferably, a peptide variant of the neuroprotective peptides of this disclosure will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to the amino acid sequence of QFQKNKLRINRQHSYD (SEQ ID NO:5), or the amino acid sequence of

(SEQ ID NO: 6)
QKNKLRINRQHS.

In some embodiments, the peptide does not comprise an amino acid sequence that is 100% identical to the amino acid sequence of SEQ ID NO:3. In some embodiments, the variant peptide(s) will have no more than 1, 2, 3, 4, 5, 6, 7, 8, 9, 10, 11, 12, 13, 14, 15, 16, 17, 18, 19, 20, 21, 22, 23, 24, 25, or 26 amino acid substitutions as compared to the amino acid sequence of QFQKNKLRINRQHSYD (SEQ ID NO:5), or the amino acid sequence of QKNKLRINRQHS (SEQ ID NO:6).

In some embodiments, the peptide comprises a dimer, a trimer, a tetramer, or a multimer of the amino acid sequence of QFQKNKLRINRQHSYD (SEQ ID NO:5), or the amino acid sequence of QKNKLRINRQHS (SEQ ID NO:6).

In another aspect, the present invention provides a cell permeable peptide having the sequence of AQKKNRNKLRRQHSY (SEQ ID NO:7), or the amino acid sequence of KKNRNKLRRQH (SEQ ID NO:8), or a multimer, derivative, or variant thereof.

In some embodiments, disclosed herein are peptides that are at least 50% identical (e.g., have at least 60%, 70%, 80%, 90%, 95% or more sequence identity) to the amino acid sequence of AQKKNRNKLRRQHSY (SEQ ID NO:7), or the amino acid sequence of KKNRNKLRRQH (SEQ ID NO:8), and that retain at least one neuroprotective property thereof. Preferably, a peptide variant of the neuroprotective peptides of this disclosure will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to the amino acid sequence of AQKKNRNKLRRQHSY (SEQ ID NO:7), or the amino acid sequence of

(SEQ ID NO: 8)
KKNRNKLRRQH.

In some embodiments, the peptide does not comprise an amino acid sequence that is 100% identical to the amino acid sequence of SEQ ID NO:3. In some embodiments, the variant peptide(s) will have no more than 1, 2, 3, 4, 5, 6, 7, 8, 9, 10, 11, 12, 13, 14, 15, 16, 17, 18, 19, 20, 21, 22, 23, 24, 25, or 26 amino acid substitutions as compared to the amino acid sequence of AQKKNRNKLRRQHSY (SEQ ID NO:7), or the amino acid sequence of KKNRNKLRRQH (SEQ ID NO:8).

In some embodiments, the peptide comprises a dimer, a trimer, a tetramer, or a multimer of the amino acid sequence of AQKKNRNKLRRQHSY (SEQ ID NO:7), or the amino acid sequence of KKNRNKLRRQH (SEQ ID NO:8).

Substituted amino acid residues may be unrelated to the amino acid residue being replaced (e.g., unrelated in terms or hydrophobicity/hydrophilicity, size, charge, polarity, etc.), or the substituted amino acid residues may constitute similar, conservative, or highly conservative amino acid substitutions. As used herein, “similar,” “conservative,” and “highly conservative” amino acid substitutions are defined as shown in the table below. The determination of whether an amino acid residue substitution is similar, conservative, or highly conservative is based exclusively on the side chain of the amino acid residue and not the peptide backbone, which may be modified to increase peptide stability, as discussed below.

| | | | Highly |
|---|---|---|---|
| | Similar Amino | Conservative | Conservative |
| | Acid | Amino Acid | Amino Acid |
| Amino Acid | Substitutions | Substitutions | Substitutions |
| Glycine (G) | A, S, N | A | n/a |
| Alanine (A) | S, G, T, V, C, P, | S, G, T | S |
| | Q | | |
| Serine (S) | T, A, N, G, Q | T, A, N | T, A |
| Threonine (T) | S, A, V, N, M | S, A V, N | S |
| Cysteine (C) | A, S, T, V, I | A | n/a |
| Proline (P) | A, S, T | A | n/a |
| Methionine (M) | L, I, V, F | L, I, V | L, I |
| Valine (V) | I, L, M, T, A | I, L, M | I |
| Leucine (L) | M, I, V, F, T, A | M, I, V, F | M, I |
| Isoleucine (I) | V, L, M, F, T, C | V, L, M, F, | V, L, M |
| Phenylalanine (F) | W, L, M, I, V | W, L | n/a |
| Tyrosine (Y) | F, W, H, L, I | F, W | F |
| Tryptophan (W) | F, L, V | F | n/a |
| Asparagine (N) | Q | Q | Q |
| Glutamine (Q) | N | N | N |
| Aspartic Acid (D) | E | E | E |
| Glutamic Acid € | D | D | D |
| Histidine (H) | R, K | R, K | R, K |
| Lysine (K) | R, H | R, H | R, H |
| Arginine (R) | K, H | K, H | K, H |

Conservative amino acid substitutions in the context of a subject peptide are selected so as to preserve activity of the peptide.

In some embodiments, the peptide is an isolated peptide. The isolated peptides of the invention can be made intracellularly in cells by introducing into the cells an expression vector encoding the peptide. Such expression vectors can be made by standard techniques. In some embodiments, the expression vector comprises a nucleic acid molecule encoding the peptide of the invention, which is operably linked to a control sequence for the expression of the peptide. The peptide can be expressed in intracellularly as a fusion with another protein or peptide (e.g., a GST fusion).

Alternative to recombinant synthesis of the peptides in the cells, the peptides can be a synthetic peptide, e.g., made by chemical synthesis using standard peptide synthesis techniques. Synthesized peptides can then be introduced into cells by a variety of means known in the art for introducing peptides into cells (e.g., liposome and the like).

### Modified Peptides of the Invention

Also contemplated in the context of the inventive methods and compositions is the modification of any neuroprotective peptides described herein, by chemical or genetic means. Examples of such modification include construction of peptides of partial or complete sequence with non-natural amino acids and/or natural amino acids in L or D enantiomeric forms. For example, any of the peptides disclosed herein, and any variants thereof, could be produced in an all-D form. Furthermore, the peptides may be modified to contain carbohydrate or lipid moieties, such as sugars or fatty acids, covalently linked to the side chains or the N- or C-termini of the amino acids. In addition, the disclosed peptides may be modified by glycosylation and/or phosphorylation.

In addition, disclosed peptides may be modified to enhance solubility and/or half-life upon being administered. For example, polyethylene glycol (PEG) and related polymers have been used to enhance solubility and the half-life of protein therapeutics in the blood. Accordingly, the disclosed peptides may be modified by PEG polymers and the like. PEG or PEG polymers means a residue containing poly(ethylene glycol) as an essential part. Such a PEG can contain further chemical groups which are necessary for the therapeutic activity of the peptides of this disclosure; which results from the chemical synthesis of the molecule; or which is a spacer for optimal distance of the parts of the molecule from one another. In addition, such a PEG can consist of one or more PEG sidechains which are linked together. PEG groups with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEGs usually have 2 to 8 arms and are described in, for example, U.S. Pat. No. 5,932,462. Especially preferred are PEGs with two PEG side-chains (PEG2) linked via the primary amino groups of a lysine (Monfardini et al., Bioconjugate Chem. 6 (1995) 62-69). The term “PEG” is used broadly to encompass any polyethylene glycol molecule, wherein the number of ethylene glycol (EG) units is at least 460, preferably 460 to 2300 and especially preferably 460 to 1840 (230 EG units refers to a molecular weight of about 10 kDa). The upper number of EG units is only limited by solubility of the PEGylated peptides of this disclosure. Usually PEGs which are larger than PEGs containing 2300 units are not used. Preferably, a PEG used in the invention terminates on one end with hydroxy or methoxy(methoxy PEG, mPEG) and is on the other end covalently attached to a linker moiety via an ether oxygen bond. The polymer is either linear or branched. Branched PEGs are e.g., described in Veronese et al., Journal of Bioactive and Compatible Polymers 12 (1997) 196-207. Suitable processes and preferred reagents to produce PEGylated peptides and variants of this disclosure are described in US Patent Pub. No. 2006/0154865. It is understood that modifications, for example, based on the methods described by Veronese, F. M., Biomaterials 22 (2001) 405-17, can be made in the procedures so long as the process results in PEGylated peptides of this disclosure. Particularly preferred processes for the preparation of PEGylated peptides of this disclosure are described in US 2008/01 19409, which is incorporated herein by reference.

Additionally, the peptides of this disclosure may be is fused to one or more domains of an Fc region of human IgG. Antibodies comprise two functionally independent parts, a variable domain known as “Fab,” that binds an antigen, and a constant domain known as “Fc,” that is involved in effector functions such as complement activation and attack by phagocytic cells. An Fc has a long serum half-life, whereas a Fab is short-lived (Capon et al., 1989, Nature 337:525-31). When constructed together with a therapeutic protein of this disclosure, an Fc domain can provide longer half-life or incorporate such functions as Fc receptor binding, protein A binding, complement fixation, and perhaps even blood-brain barrier, or placental transfer. In one example, a human IgG hinge, CH2, and CH3 region may be fused at either the amino-terminus or carboxyl-terminus of the peptides of this disclosure using methods known to the skilled artisan. The resulting fusion polypeptide may be purified by use of a Protein A affinity column. Peptides and proteins fused to an Fc region have been found to exhibit a substantially greater half-life in vivo than the unfused counterpart. Also, a fusion to an Fc region allows for dimerization/multimerization of the fusion polypeptide. The Fc region may be a naturally occurring Fc region, or may be altered to improve certain qualities, such as therapeutic qualities, circulation time, or reduced aggregation.

The peptides may also be modified to contain sulfur, phosphorous, halogens, metals, etc. Amino acid mimics may be used to produce polypeptides, and therefore, the polypeptides of this disclosure may include amino acid mimics that have enhanced properties, such as resistance to degradation. For example, the polypeptides may include one or more (e.g., all) peptide monomers.

### IV. Screening Assays

The invention further provides methods (also referred herein as “screening assays”) for identifying compounds, e.g., candidate or test compounds (e.g., peptides, small molecules or other drugs) which modulate, e.g., inhibit or stimulate, the activity of TRPM2, e.g., interaction between TRPM2 and NMDAR, and for testing or optimizing the activity of modulators.

Agents that are capable of modulating the activity of TRMP2 or its binding ligands, e.g., NMDAR, as identified by the methods of the invention, are useful as candidate compounds useful for treating or preventing neurological injury or neurological disorder in a subject in need thereof. For example, in one aspect, the present invention provides methods for identifying a compound useful for treating or preventing neurological injury or neurological disorder. The methods include providing a test compound (or a plurality of test compounds), determining the effect of the test compound on the interaction between TRPM2 and NMDAR, and selecting a compound which modulates, e.g., decreases, the interaction between TRPM2 and NMDAR, thereby identifying a compound useful for treating or preventing neurological injury or neurological disorder.

In some embodiments, the compound targets a NMDAR-binding site on TRPM2. A compound can target the NMDAR-binding site by binding to the NMDAR-bindign site, thus blocking interaction between TRPM2 and NMDAR. A compound can also target the NMDAR-binding site by mimicing the amino acid sequence of NMDAR-binding site, thus competing with NMDAR for TRPM2 binding.

In some embodiments, the compound targets a TRPM2-binding site on NMDAR. A compound can target the TRPM2-binding site by binding to the TRPM2-binding site, thus blocking interaction between TRPM2 and NMDAR. A compound can also target the TRPM2-binding site by mimicking the amino acid sequence of TRPM2-binding site, thus competing with NMDAR for TRPM2 binding.

Examples of agents, candidate compounds or test compounds include, but are not limited to, proteins, peptides, peptidomimetics, small molecules, nucleic acids (e.g., DNA and RNA), carbohydrates, lipids, and other drugs.

Compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. Nos. 5,738,996; and 5,807,683, the entire contents of each of the foregoing references are incorporated herein by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, the entire contents of each of the foregoing references are incorporated herein by reference. Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310). The entire contents of each of the foregoing references are incorporated herein by reference.

The test compound can be contacted with a cell that expresses the TRPM2 protein or a molecule with which TRPM2 directly interacts, e.g., NMDAR. For example, the test compound can be contacted with a cell that naturally expresses or has been engineered to express the protein(s) by introducing into the cell an expression vector encoding the protein.

Alternatively, the test compounds can be subjected to a cell-free composition that includes the protein(s) (e.g., a cell extract or a composition that includes e.g., purified natural or recombinant protein).

Compounds that modulate the activity of TRPM2, or a binding ligand of TRPM2, e.g., NMDAR, can be identified using various “read-outs.” For example, a cell can be transfected with an expression vector, incubated in the presence and in the absence of a test compound, and the effect of the compound on the interaction between TRPM2 and NMDAR or on a biological response regulated by TRPM2 can be determined. The biological activities of TRPM2 include activities determined in vivo, or in vitro, according to standard techniques. Activity can be a direct activity, such as an association with a binding ligand, e.g., NMDAR. Alternatively, the activity is an indirect activity, such as a change in calcium influx in neurons.

To determine whether a test compound modulates TRPM2 protein expression, in vitro transcriptional assays can be performed. To determine whether a test compound modulates TRPM2 mRNA expression, various methodologies can be performed, such as quantitative or real-time PCR.

A variety of reporter genes are known in the art and are suitable for use in the screening assays of the invention. Examples of suitable reporter genes include those which encode chloramphenicol acetyltransferase, beta-galactosidase, alkaline phosphatase, green fluorescent protein, or luciferase. Standard methods for measuring the activity of these gene products are known in the art.

A variety of cell types are suitable for use as an indicator cell in the screening assay. Preferably a cell line is used which expresses low levels of endogenous TRPM2 and is then engineered to express recombinant protein. Cells for use in the subject assays include eukaryotic cells. For example, in one embodiment, a cell is a fungal cell, such as a yeast cell. In another embodiment, a cell is a plant cell. In yet another embodiment, a cell is a vertebrate cell, e.g., an avian cell or a mammalian cell (e.g., a murine cell, or a human cell).

Recombinant expression vectors that can be used for expression of, e.g., TRPM2, are known in the art. For example, the cDNA is first introduced into a recombinant expression vector using standard molecular biology techniques. A cDNA can be obtained, for example, by amplification using the polymerase chain reaction (PCR) or by screening an appropriate cDNA library. The nucleotide sequences of cDNAs for or a molecule in a signal transduction pathway involving (e.g., human, murine and yeast) are known in the art and can be used for the design of PCR primers that allow for amplification of a cDNA by standard PCR methods or for the design of a hybridization probe that can be used to screen a cDNA library using standard hybridization methods.

In another embodiment, the test compounds can be subjected to a cell-free composition that includes the protein(s) (e.g., a cell extract or a composition that includes e.g., either purified natural or recombinant protein). TRPM2 expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment, compounds that specifically modulate TRPM2 activity or the activity of a binding ligand in a signal transduction pathway involving TRPM2 are identified based on their ability to modulate the interaction of TRPM2 with its binding ligand, e.g., NMDAR. Suitable assays are known in the art that allow for the detection of protein-protein interactions (e.g., immunoprecipitations, two-hybrid assays and the like). By performing such assays in the presence and absence of test compounds, these assays can be used to identify compounds that modulate (e.g., inhibit or enhance) the activity of TRPM2 with a binding ligand, e.g., NMDAR.

Compounds identified in the subject screening assays can be used in methods of modulating one or more of the biological responses regulated by TRPM2. It will be understood that it may be desirable to formulate such compound(s) as pharmaceutical compositions as described herein prior to contacting them with cells.

Once a test compound is identified that directly or indirectly modulates TRPM2 expression or activity by one of the variety of methods described hereinbefore, the selected test compound (or “compound of interest”) can then be further evaluated for its effect on cells, for example by contacting the compound of interest with cells either in vivo (e.g., by administering the compound of interest to an organism) or ex vivo (e.g., by isolating cells from an organism and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

In another aspect, the invention pertains to a combination of two or more of the assays described herein. For example, a modulator can be identified using a cell-based or a cell-free assay, and the ability of the modulators to increase or decrease the interaction between TRPM2 and NMDAR can be confirmed in vivo, e.g., in an animal, such as, for example, an animal model for, e.g., a mouse model of middle cerebral artery occlusion.

Moreover, a modulator of TRPM2 can be used in an animal model to determine the efficacy, toxicity, or side effects of treatment with such a modulator. Alternatively, a modulator identified as described herein can be used in an animal model to determine the mechanism of action of such a modulator.

In another embodiment, it will be understood that similar screening assays can be used to identify compounds that indirectly modulate the activity of TRPM2, e.g., by performing screening assays such as those described above using molecules with which TRPM2 interacts, e.g., NMDAR, or any molecules that act either upstream or downstream of TRPM2 in the pathway.

Compounds identified by the screening assays of the present invention are considered as candidate therapeutic compounds useful for treating diseases, e.g., neurological injury or disorders, e.g., stroke, as described herein. Thus, the invention also includes compounds identified in the screening assays, and methods for their administration and use in the treatment, prevention, or delay of development or progression of diseases described herein.

### V. Pharmaceutical Compositions

The present invention also provides pharmaceutical compositions comprising the agents or the peptides of the invention for use in treating neurological injuries, diseases, and disorders. The pharmaceutical compositions may be formulated according to any of the conventional methods known in the art and widely described in the literature. Thus, the active ingredient (e.g., an agent, e.g., a peptide, of this disclosure) may be incorporated, optionally together with other active substances, with one or more conventional pharmaceutically acceptable carriers, diluents and/or excipients, etc., appropriate for the particular use of the composition, to produce conventional preparations that are suitable or may be made suitable for administration. Carriers may include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®. They may be formulated as liquids, as semi-solids or solids, liquid solutions, dispersions, suspensions, and the like, depending on the intended mode of administration and therapeutic application. In some embodiments, the inventive composition is prepared in a form of an injectable or infusible solution. Agents, e.g., peptides, of this disclosure may be formulated in a “liposome” which is a small vesicle composed of various types of lipids, phospholipids and/or surfactant which is useful for delivery of a drug (such as an inhibitory peptide of this disclosure) to a mammal. The components of the liposome are commonly arranged in a bilayer formation, similar to the lipid arrangement of biological membranes.

Compositions of this disclosure may include a carrier protein, such as serum albumin (e.g., HSA, BSA, and the like). The serum albumin may be purified or recombinantly produced. By mixing the agents of the invention, e.g., the neuroprotective polypeptide(s), in the pharmaceutical composition with serum album, the agents, e.g., the neuroprotective polypeptides, may be effectively “loaded” onto the serum albumin, allowing a greater amount of the agents to be successfully delivered to a site of neurological injury.

Methods of treating neurological injuries, diseases, or neurodegenerative diseases of this disclosure may include administration of an agent, a peptide, or a pharmaceutical composition of this disclosure via any one of a variety of routes, including intravenous (IV), intramuscular (IM), intraarterial, intramedullary, intrathecal, subcutaneous (SQ), intraventricular, transdermal, interdermal, intradermal, by intratracheal instillation, bronchial instillation, and/or inhalation; as a nasal spray, and/or aerosol, and/or through a portal vein catheter. Any appropriate site of administration may be used. For example, the composition may be administered locally and directly at the site where action is required or may be attached or otherwise associated, e.g. conjugated, with entities which will facilitate the targeting to an appropriate location in the body.

In these compositions, any physiologically compatible carrier, excipient, diluent, buffer or stabilizer may be used. Examples of suitable carriers, excipients, diluents, buffers and stabilizers include one or more of water, saline, phosphate buffered saline, dextrose, glycerol, ethanol and the like, as well as combinations thereof. In some cases, isotonic agents, e.g., sugars, polyalcohols (e.g., mannitol, sorbitol), or sodium chloride may be included. In certain embodiments, the compositions of this disclosure may be formulated so as to provide quick, sustained, or delayed release of the active ingredient (peptides of this disclosure, or variants thereof and/or additional drug(s)) after administration to the subject by employing procedures well known in the art. As described above, in certain embodiments, the composition is in a form suitable for injection and suitable carriers may be present at any appropriate concentration, but exemplary concentrations are from 1% to 20%, or from 5% to 10%.

Therapeutic compositions typically must be sterile and stable under conditions of manufacture and storage. Appropriate ways of achieving such sterility and stability are well known and described in the art.

Pharmaceutical compositions are typically formulated in unit dosage form for case of administration and uniformity of dosage. It will be understood, however, that the total daily (or other) usage of the compositions of the present disclosure will be decided by the attending physician within the scope of sound medical judgment. The specific therapeutically-effective dosage level for any particular subject will depend upon a variety of factors including the activity of the composition employed; the half-life of the composition after administration; the age, body weight, general health, sex, and diet of the subject; the time of administration, route of administration, and rate of excretion of the agent, e.g., the peptide, and (if used) the additional therapeutic agent employed; the duration of the treatment; drugs used in combination or coincidental with the specific compound employed; and like factors, well known in the medical arts. Furthermore, effective doses may be extrapolated from dose-response curves derived from in vitro and/or in vivo animal models.

Thus, suitable doses of the agent of this disclosure and other active ingredients (if included) will vary from patient to patient and will also depend on the severity/stage of the stroke. In some embodiments, said dosages constitute a therapeutically effective amount or a prophylactically effective amount, depending on the nature of the treatment involved. In related embodiments, the dosages constitute a neuro-restorative- or rehabilitation-enhancing amount. The ability of the agent to elicit a desired response in the individual will also be a factor. Exemplary daily doses are: 0.1 mg/kg to 250 mg/kg, or 0.1 mg/kg to 200 mg/kg or 100 mg/kg, or 0.5 mg/kg to 100 mg/kg, or 1 mg/kg to 50 mg/kg or 1 mg/kg to 10 mg/kg, of the active ingredient. This may be administered as a single unit dose or as multiple unit doses administered more than once a day, for example, subcutaneously, intraperitoneally, or intravenously. It is to be noted, however, that appropriate dosages may vary depending on the patient, and that for any particular subject, specific dosage regimes should be adjusted over time according to the individual needs of the patient. For example, the dosage and administration protocol may be adjusted over time, or with patient advances in rehabilitation to less than once daily, including for example, every other day, three times weekly, or two times weekly, or once weekly, or every other week, etc. Thus, the dosage ranges set forth herein are to be regarded as exemplary and are not intended to limit the scope or practice of the claimed compositions or methods.

### VI. Kits

In one aspect, this disclosure further provides kits for the treatment of neurological injury, neurological diseases, or neurodegenerative diseases comprising an agent, e.g., a peptide, of this disclosure, or variants thereof, or a composition comprising the same. Kits may include one or more other elements including, but not limited to, instructions for use; other therapeutic agents (i.e., for combination or emergency therapy of stroke); other reagents, e.g., a diluent, devices or other materials for preparing composition for administration; pharmaceutically acceptable carriers; and devices or other materials for administration to a subject. Instructions for use may include instructions for therapeutic application, including suggested dosages and/or modes of administration, e.g., in a human subject, as described herein. In some embodiments, the kits are for use in the methods and uses as described herein, e.g. therapeutic, diagnostic, or imaging methods, or are for use in in vitro assays or methods.

In some embodiments, the kits are for diagnosing neurological diseases, disorders or impairments and optionally comprise instructions for use of the kit components to diagnose or evaluate the severity of such neurological diseases, disorders or impairments.

It is to be understood that this invention is not limited to particular assay methods, or test agents and experimental conditions described, as such methods and agents may vary. It is also to be understood that the terminology used herein is for the purpose of describing particular embodiments only, and is not intended to be limiting.

The present invention is further illustrated by the following examples, which are not intended to be limiting in any way. The entire contents of all references, patents and published patent applications cited throughout this application, as well as the Figures, are hereby incorporated herein by reference.

### EXAMPLES

### Example 1: Functional Coupling of TRPM2 and NMDARs Exacerbates Excitotoxicity in Ischemic Brain Injury

In this example, the enhanced NMDAR's extrasynaptic excitotoxicity upon ischemic insults is revealed through a physical and functional coupling of TRPM2 to NMDARs, and the resulting increase in surface expression of NMDARs, which is mediated by interaction of PKCγ with TRPM2. A specific NMDAR-interacting domain on TRPM2 is identified. A cell-permeable peptide, TAT-EE3, is developed. TAT-EE3 is demonstrated to protect neurons against ischemic injury in vitro and in vivo. These findings provide an unconventional targetable strategy to inhibit ischemic injury by targeting TRPM2, which indirectly eliminates NMDARs' excitotoxicity.

### Results

### TRPM2 Deletion in Neurons Prevents Ischemic Injury and Protects the Brain Against Ischemic Stroke

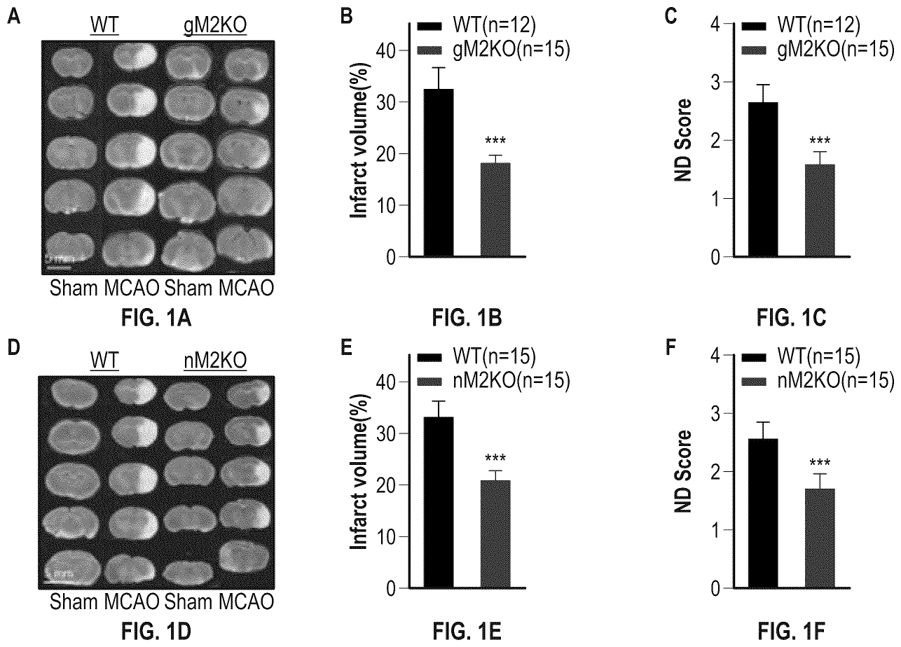

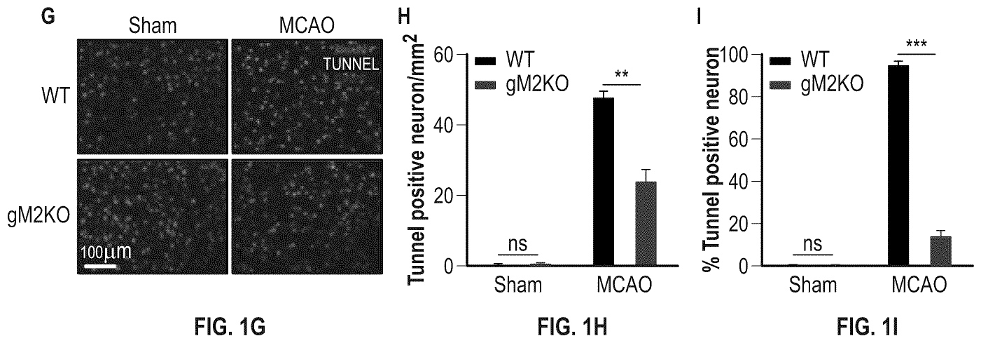

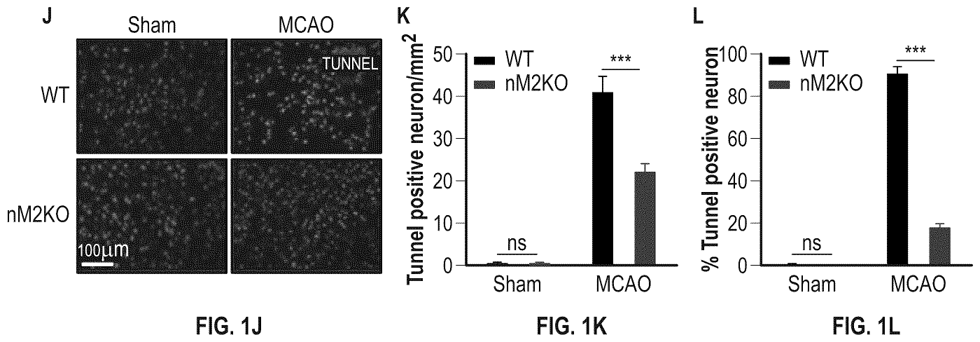

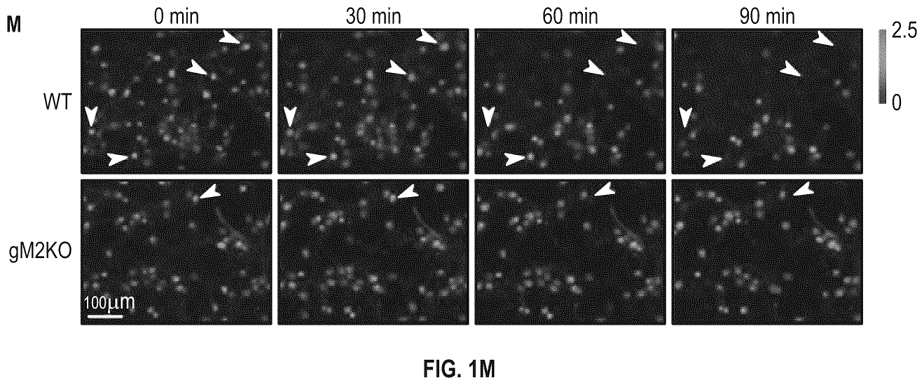

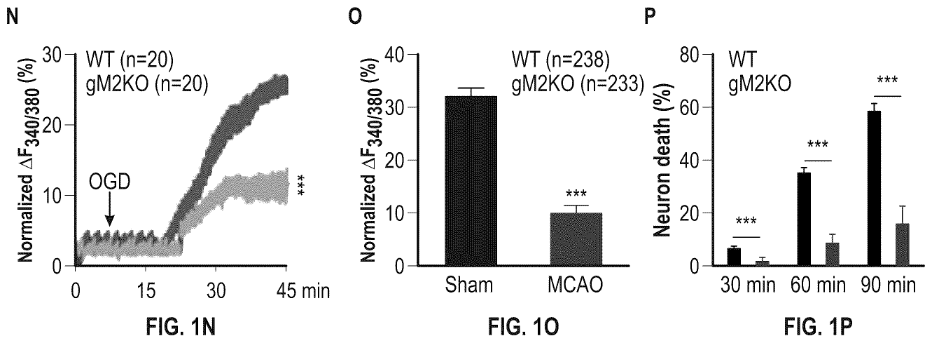

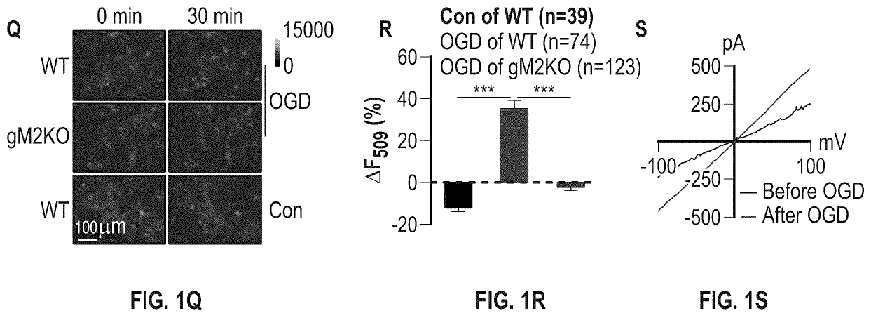

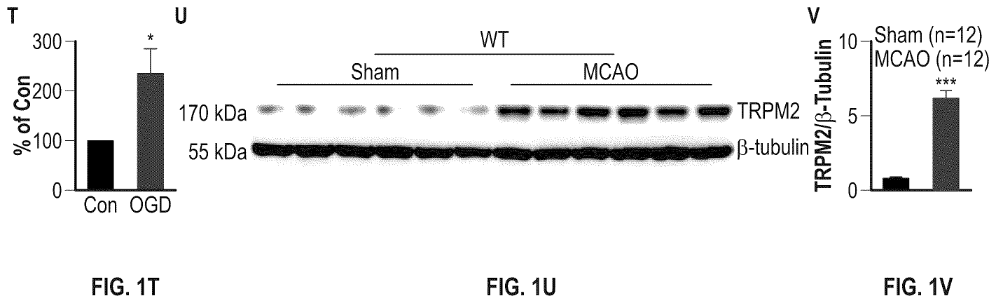

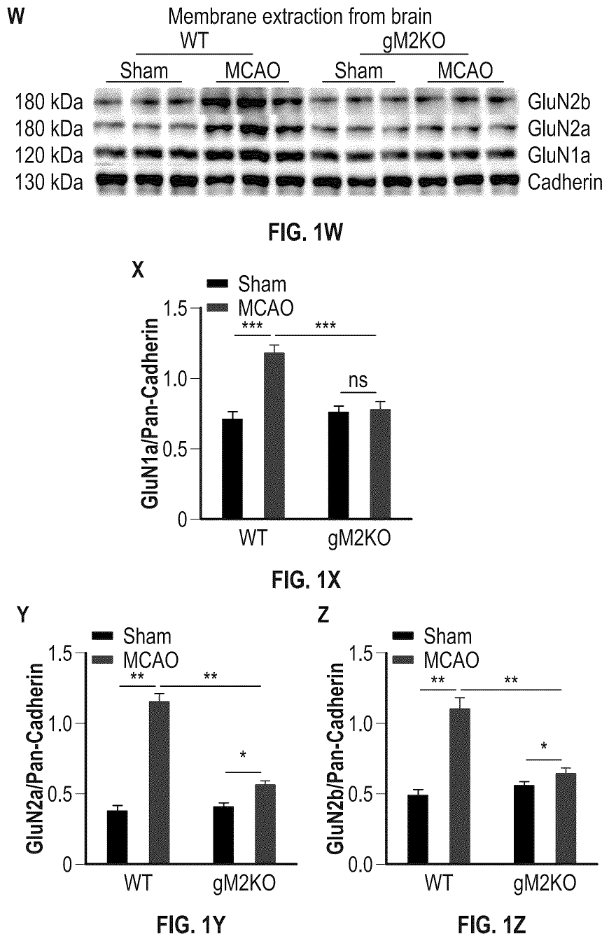

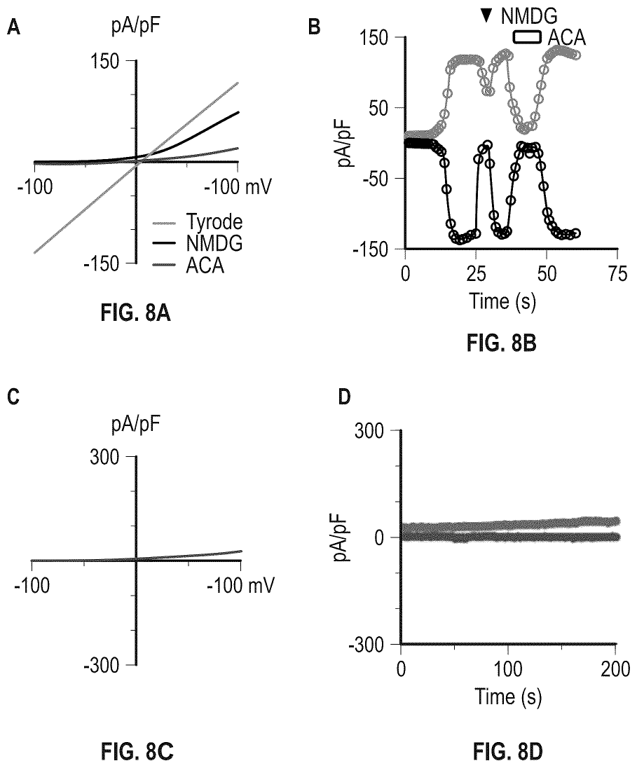

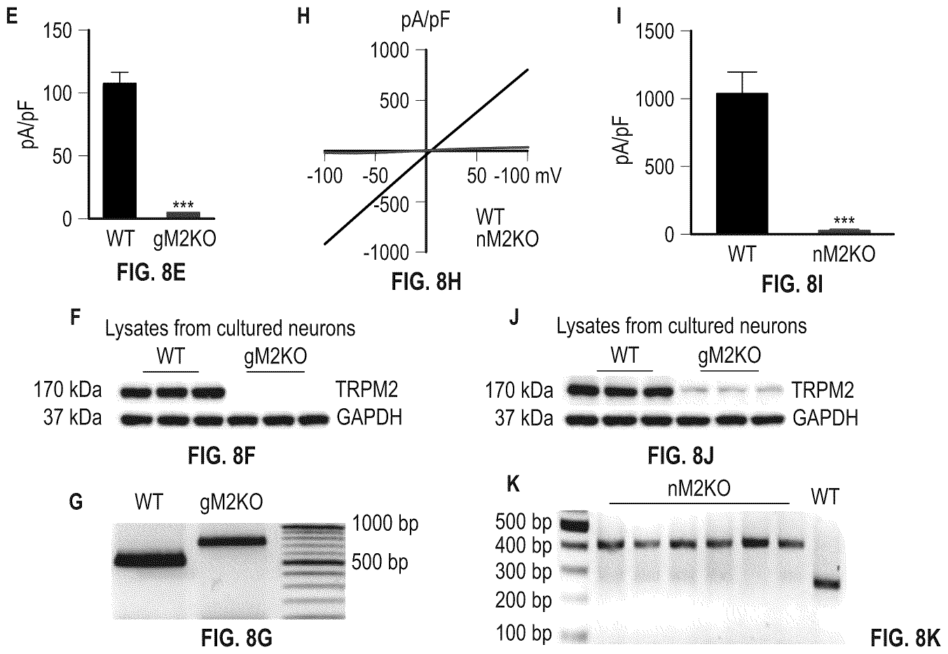

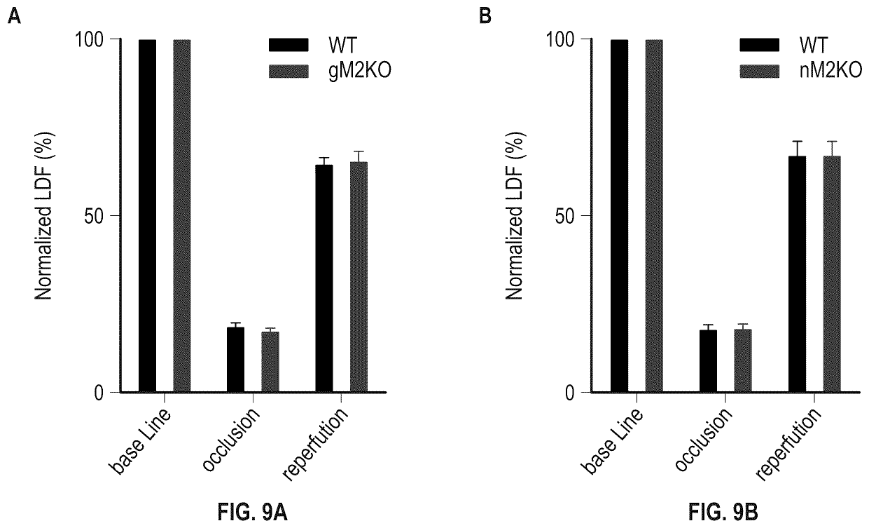

The oxidative stress-activated TRPM2 is expressed in various types of cells (Fonfria, E., Murdock, P. R., Cusdin, F. S., Benham, C. D., Kelsell, R. E., and McNulty, S. (2006). Tissue distribution profiles of the human TRPM cation channel family. Journal of receptor and signal transduction research 26, 159-78). Inhibition of TRPM2 attenuates ischemic injury, yet the mechanism by which TRPM2 leads to deleterious effects is not fully understood, as both neurons and immune cells were suggested as the primary cause of ischemic injury mediated by TRPM2 (Alim, I., Teves, L., Li, R., Mori, Y., and Tymianski, M. (2013). Modulation of NMDAR subunit expression by TRPM2 channels regulates neuronal vulnerability to ischemic cell death. The Journal of neuroscience: the official journal of the Society for Neuroscience 33, 17264-77; Gelderblom, M., Melzer, N., Schattling, B., Gob, E., Hicking, G., Arunachalam, P., Bittner, S., Ufer, F., Herrmann, A. M., Bernreuther, C., et al. (2014). Transient receptor potential melastatin subfamily member 2 cation channel regulates detrimental immune cell invasion in ischemic stroke. Stroke; a journal of cerebral circulation 45, 3395-02). To elucidate the underlying mechanisms, neuron-specific Trpm2 deletion was established using nestin-cre mice crossed with TRPM2fl/fl mice, to determine neuronal damage mediated by TRPM2 during ischemic stroke. Global knockout of TRPM2 (TRPM2-KO) was used as a comparison. Trpm2 deletion was confirmed by detecting TRPM2 protein expression and functional current recording (FIGS. 8A-8K). Using a 120-min middle cerebral artery occlusion followed by reperfusion (MCAO), infarct volume was evaluated 24 hrs after MCAO by TTC staining. Successful MCAO was confirmed by monitoring blood flow reduction by 85% (FIGS. 9A and 9B). Similar to the protective effects produced by globally Trpm2 knockout (gM2KO) as previously reported (Alim, I., Teves, L., Li, R., Mori, Y., and Tymianski, M. (2013). Modulation of NMDAR subunit expression by TRPM2 channels regulates neuronal vulnerability to ischemic cell death. The Journal of neuroscience: the official journal of the Society for Neuroscience 33, 17264-77) (FIGS. 1A-C), neuron-specific Trpm2 deletion (Cre+, TRPM2fl/fl; nM2KO) exhibited significantly reduced infarct volume and markedly improved neurological performance in comparison with WT (Cre−, TRPM2fl/fl; WT) littermates (FIGS. 1D-F). These results establish that TRPM2 in the neuron plays a key role in mediating neuronal cell death. To further determine the mechanisms of TRPM2 mediated neuronal death during ischemic stroke, neuronal cell death was evaluated by tunnel staining. At the ischemic penumbra, the number of tunnel positive neurons was significantly smaller in global TRPM2-KO and neuron specific TRPM2-KO brain slices than those of WT littermates (FIGS. 1G-L), indicating that attenuation of apoptosis by Trpm2 deletion mediates protective effects against ischemic stroke.

### TRPM2 Enhances Surface Expression of NMDARs and Exacerbates Excitotoxicity

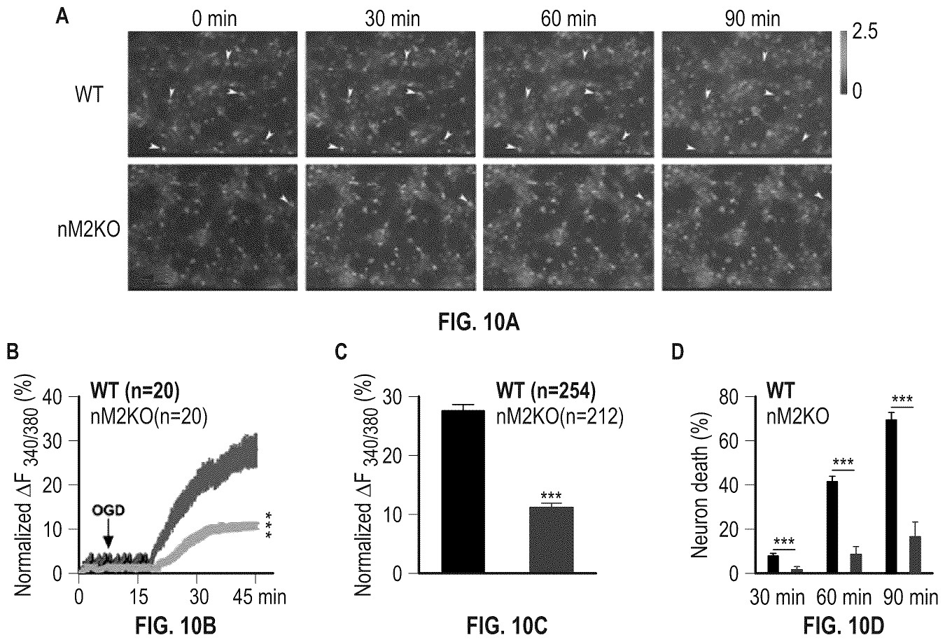

Various mechanisms are involved in neuronal death, among which Ca2+ overload is a major factor. Using cultured cortical neurons, oxygen-glucose deprivation (OGD) conditions were applied to the neurons to mimic in vivo ischemic injury, and analyzed changes of intracellular Ca2+ and cell death as previously reported (Weilinger, N. L., Lohman, A. W., Rakai, B. D., Ma, E. M., Bialecki, J., Maslicieva, V., Rilea, T., Bandet, M. V., Ikuta, N. T., Scott, L., et al. (2016). Metabotropic NMDA receptor signaling couples Src family kinases to pannexin-1 during excitotoxicity. Nature neuroscience 19, 432-42). OGD induced a persistent rise of intracellular Ca2+ (FIGS. 1M-O) until the lysis of neurons as reflected by a complete loss of Fura-2 fluorescence. The lysed neurons were counted as dead neurons as neuronal lysis is a hallmark feature of necrosis (Weilinger, N. L., Lohman, A. W., Rakai, B. D., Ma, E. M., Bialecki, J., Maslicieva, V., Rilea, T., Bandet, M. V., Ikuta, N. T., Scott, L., et al. (2016). Metabotropic NMDA receptor signaling couples Src family kinases to pannexin-1 during excitotoxicity. Nature neuroscience 19, 432-42). Throughout the 90 mins of OGD perfusion, a noticeable number of neurons (6.8%) in the WT group died at 30 mins OGD, and the percentage of neuron death increased to 35.2% and 58.5% at 60 and 90 mins, respectively (FIGS. 1M-P). In contrast, there was only 1.9%, 8.7% and 16.3% dead neurons at 30, 60 and 90 mins in the TRPM2-KO group, respectively (FIG. 1P), indicating that TRPM2 deletion protects neuron against OGD induced neuronal death. Similar results were also observed in neurons isolated from neuron-specific Trpm2 deletion Cre+ mice in comparison with Cre-control littermates (FIGS. 10A-10D). Consistent with the higher percentage of neuronal death induced by OGD, the increase in intracellular Ca2+ was also remarkably higher in WT than in TRPM2-KO neurons (FIGS. 1M-O). It was previously shown that during in vitro ischemia of cultured cortical neuron, 80% of Ca2+ entry is mediated by NMDARs (Goldberg and Choi, 1993; Lipton, 1999). The drastic reduction of Ca2+ entry by Trpm2 deletion (FIG. 1M-P) suggests that TRPM2 might have affected NMDAR functions during OGD exposure.

Mitochondrial dysfunction is another hallmark of excitotoxicity and an early event leading to neuronal death (Keelan, J., Vergun, O., and Duchen, M. R. (1999). Excitotoxic mitochondrial depolarisation requires both calcium and nitric oxide in rat hippocampal neurons. The Journal of physiology 520 Pt 3, 797-813; Schinder, A. F., Olson, E. C., Spitzer, N.C., and Montal, M. (1996). Mitochondrial dysfunction is a primary event in glutamate neurotoxicity. The Journal of neuroscience: the official journal of the Society for Neuroscience 16, 6125-33, 1996; Vergun, O., Keclan, J., Khodorov, B. I., and Duchen, M. R. (1999). Glutamate-induced mitochondrial depolarisation and perturbation of calcium homeostasis in cultured rat hippocampal neurones. The Journal of physiology 519 Pt 2, 451-66; White, R. J., and Reynolds, I. J. (1996). Mitochondrial depolarization in glutamate-stimulated neurons: an early signal specific to excitotoxin exposure. The Journal of neuroscience: the official journal of the Society for Neuroscience 16, 5688-97). Dysfunction of mitochondria is characterized by depolarized mitochondria membrane potential caused by opening of the mitochondrial permeability transition pore (mPTP) (Lemasters, J. J., Theruvath, T. P., Zhong, Z., and Nieminen, A. L. (2009). Mitochondrial calcium and the permeability transition in cell death. Biochimica et biophysica acta 1787, 1395-1401), which can be monitored by rhodamine 123 (Rh123) fluorescence dequenching assay (Nguyen, P. V., Marin, L., and Atwood, H. L. (1997). Synaptic physiology and mitochondrial function in crayfish tonic and phasic motor neurons. Journal of neurophysiology 78, 281-94). OGD induced mitochondrial depolarization is indicated by increased Rh123 fluorescence in WT neurons, whereas TRPM2 deletion largely prevented mitochondrial depolarization (FIGS. 1Q-R). Since activation of NMDARs is known to cause mitochondrial depolarization (Abramov, A. Y., and Duchen, M. R. (2008). Mechanisms underlying the loss of mitochondrial membrane potential in glutamate excitotoxicity. Biochimica et biophysica acta 1777, 953-64; Qiu, J., Tan, Y. W., Hagenston, A. M., Martel, M. A., Kneisel, N., Skchel, P. A., Wyllie, D. J., Bading, H., and Hardingham, G. E. (2013). Mitochondrial calcium uniporter Meu controls excitotoxicity and is transcriptionally repressed by neuroprotective nuclear calcium signals. Nature communications 4, 2034; Yan, J., Bengtson, C. P., Buchthal, B., Hagenston, A. M., and Bading, H. (2020). Coupling of NMDA receptors and TRPM4 guides discovery of unconventional neuroprotectants. Science 370), the fact that TRPM2 deletion largely eliminated mitochondrial depolarization during OGD provides another line of evidence suggesting that TRPM2 might have influenced NMDAR function.

Since TRPM2 is sensitive to oxidative stress stimuli, how TRPM2 is regulated by ischemic stroke in vivo and in vitro was investigated. Sub-optimal Ca2+ and ADPR concentrations (Du, J., Xie, J., and Yue, L. (2009) were used. Intracellular calcium activates TRPM2 and its alternative spliced isoforms. Proceedings of the National Academy of Sciences 107, 7239-44) in the pipette solution for TRPM2 recording in neurons and exposed neurons to OGD. During OGD stimulation, TRPM2 current amplitude was significantly increased (FIGS. 1S-IT), indicating an enhanced channel activity, which might also happen during in vivo ischemic stroke. Moreover, TRPM2 expression level in the MCAO brains was 7.6-fold higher than in the sham control brains (FIGS. 1U-V).

As both OGD induced neural death (FIGS. 1M-P) and mitochondrial dysfunction results (FIGS. 1Q-R) suggest that TRPM2 might influence NMDAR functions, the influence of TRPM2 on NMDAR functions in ischemic stroke in vivo was evaluated. Using plasma membrane protein extracts from brains of TRPM2-KO (gM2KO) and WT littermate mice subjected to MCAO or sham procedure, it was found that the surface expression levels of NMDARs, including GluN1, GluN2a and GluN2b, were much higher in the WT MCAO mice in comparison with sham control mice. Remarkably, the increase in the surface expression level of NMDARs induced by MCAO mice was almost totally abolished by TRPM2 deletion (FIGS. 1W-Z). This finding prompted the proposal that, as an oxidative stress sensor, TRPM2 influences NMDAR surface expression and function during ischemic stroke, thereby exacerbating NMDAR's excitotoxicity.

TRPM2 Interacts with NMDARs

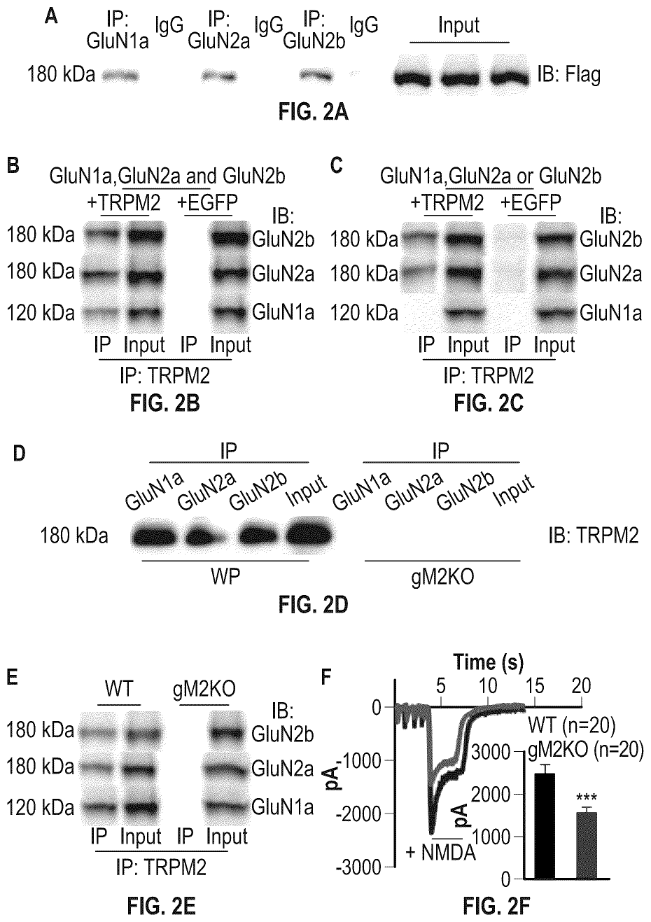

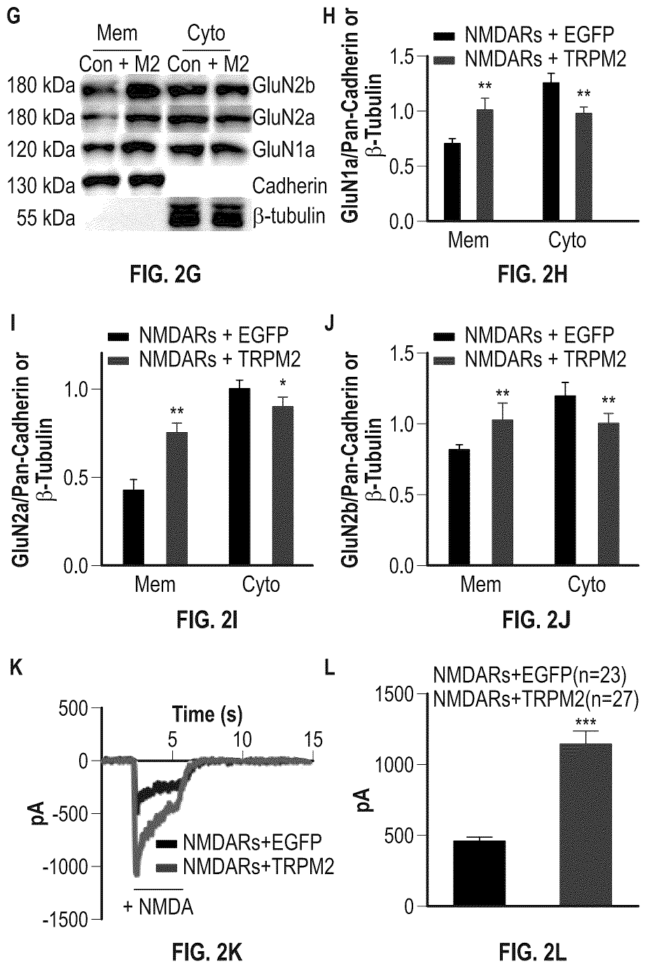

To understand how TRPM2 may influence NMDAR surface expression in MCAO brains, the interaction between TRPM2 and NMDARs was tested. HEK-293 cells heterologously expressing TRPM2 and the NMDAR subunits GluN1, GluN2a and GluN2b (GluN1/GluN2a/GluN2b) were first used; and co-immunoprecipitation (co-IP) experiments were performed. As shown in FIG. 2A, TRPM2 can be pulled down by antibodies specifically against GluN1, GluN2a and GluN2b, indicating that TRPM2 interacts with the NMDAR protein complex (FIG. 2A). In reciprocal co-IP experiments, when TRPM2 was immunoprecipitated by anti-TRPM2, GluN1, GluN2a and GluN2b were detected in the precipitated complex by WB (FIG. 2B), further indicating that TRPM2 interacted with NMDARs.

Since the subunits GluN1, GluN2a, and GluN2b form heteromeric channel complex, antibodies against any of these subunits may pull down the entire heterotetrameric complex. To determine which subunits interact with TRPM2, TRPM2 individually transfected with GluN1, GluN2a or GluN2b. As shown in FIG. 2C, TRPM2 interacted with both GluN2a and GluN2b but not GluN1 when they were separately transfected with TRPM2, indicating that in the GluN1/GluN2a/GluN2b complex, GluN2a and GluN2b subunits interact with TRPM2.

It was then sought to determine whether endogenous TRPM2 interacts with NMDARs complex. We used brain tissues from WT MCAO mice since TRPM2 is highly up-regulated by MCAO (FIGS. 1U-V). As shown in FIGS. 2D-E, GluN1, GluN2a and GluN2b were able to pull down TRPM2, and reciprocally, anti-TRPM2 was able to pull down GluN1, GluN2a and GluN2b in the WT MCAO brain lysates but not in the TRPM2-KO MCAO brain lysates. The interaction between TRPM2 and NMDARs in both exogenous expression systems and brains prompted an investigation into the functional significance of the interaction.

### Functional Coupling Between NMDARs and TRPM2

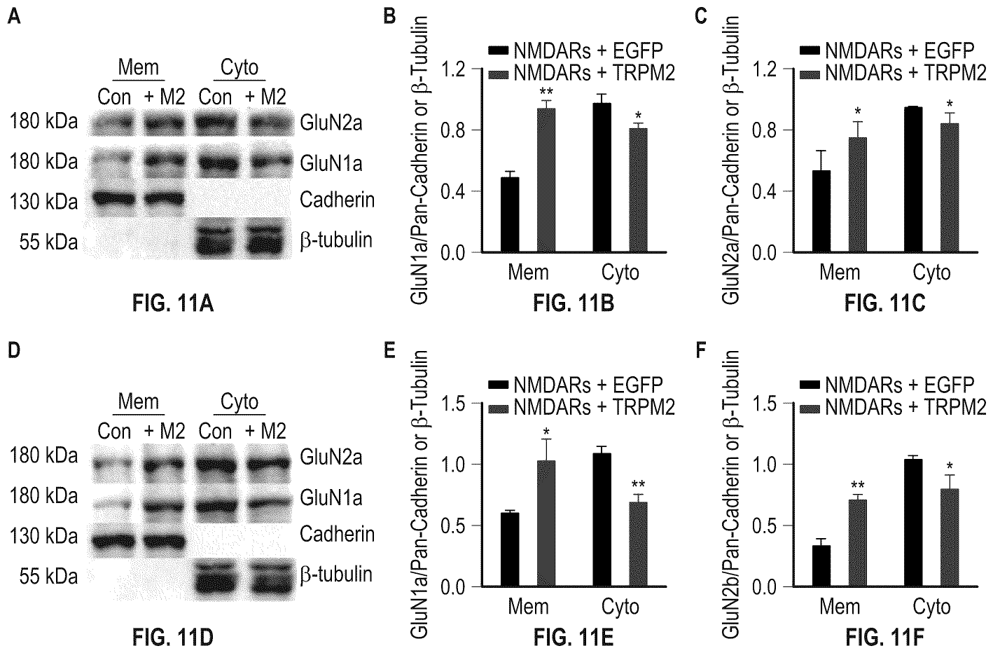

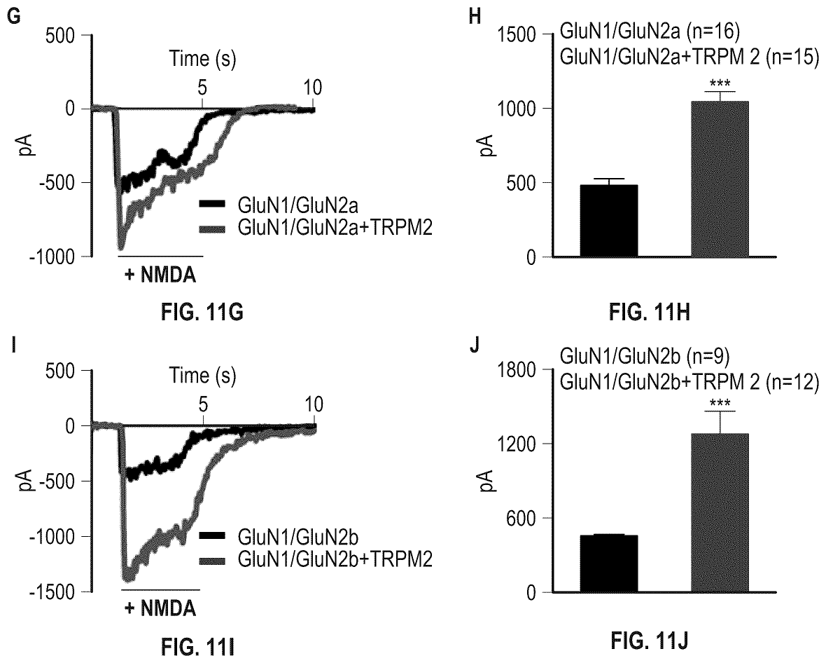

NMDAR currents in cultured neurons from WT and TRPM2-KO mice were tested. NMDAR currents elicited by 10 μM NMDA at holding potential of −80 mV (FIG. 2F) were much bigger in WT neurons than that in the TRPM2-KO neurons (FIG. 2F). The functional interaction of NMDARs and TRPM2 in the overexpression system were then tested. In HEK293T cells overexpressing NMDARs with TRPM2, surface expression levels of GluN1, GluN2a and GluN2b were higher than that in HEK293T cells overexpressing NMDARs with control plasmids EGFP vector (FIGS. 2G-J). Consistent with the enhanced surface expression levels, NMDAR currents elicited by NMDA in NMDARs/TRPM2 expressing cells were significantly larger than that in NMDARs/EGFP expressing cells (FIGS. 2K-L). The increased surface expression levels and enhanced NMDAR channel functions were also observed in separated transfections when GluN1/GluN2a or GluN1/GluN2b were co-expressed with TRPM2 (FIG. 11A-11J). These results suggest that TRPM2 and NMDRAs physical interactions result in functional coupling reflected by the enhanced functional currents of NMDARs.

### Identification of the NMDAR-Interacting Domain EE3 at the N-Terminus of TRPM2

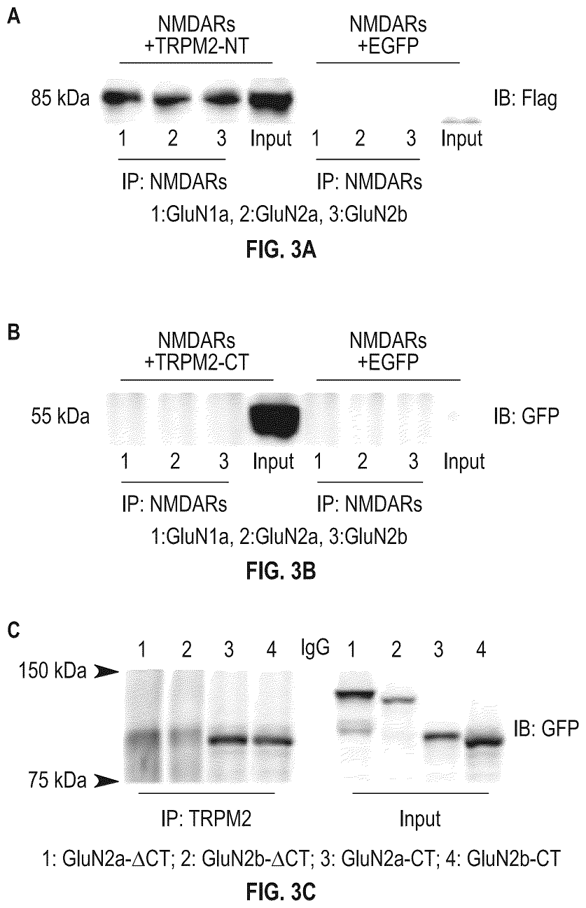

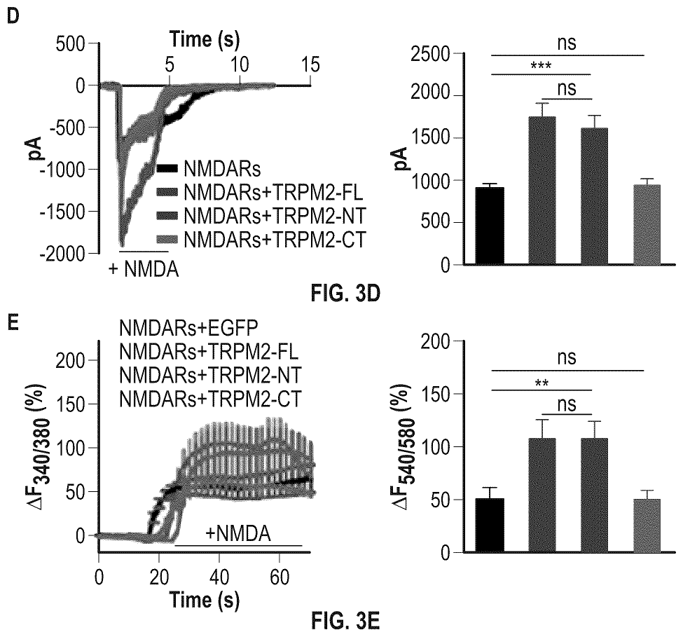

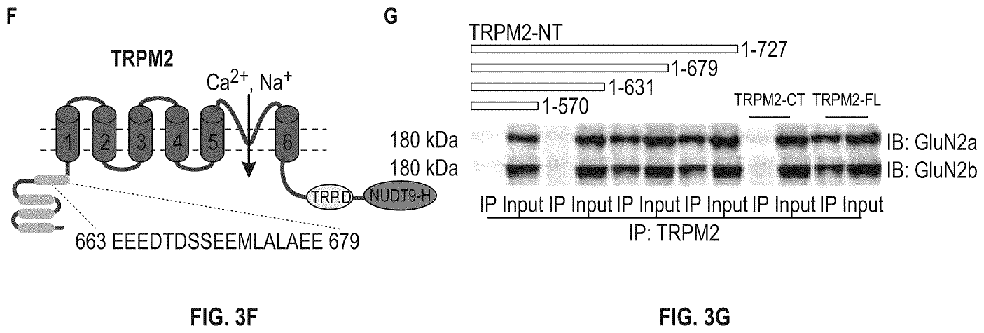

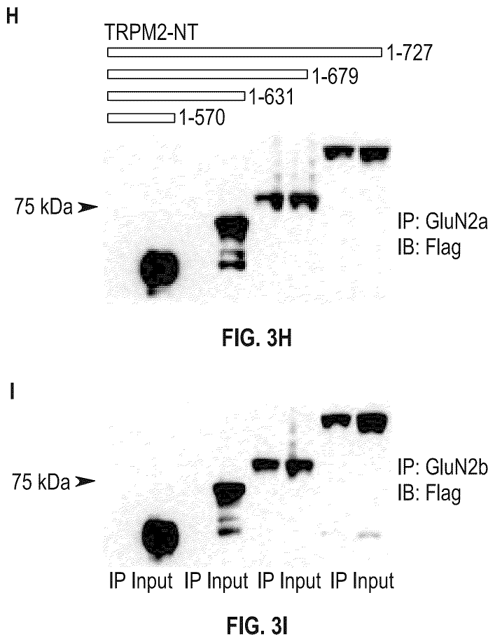

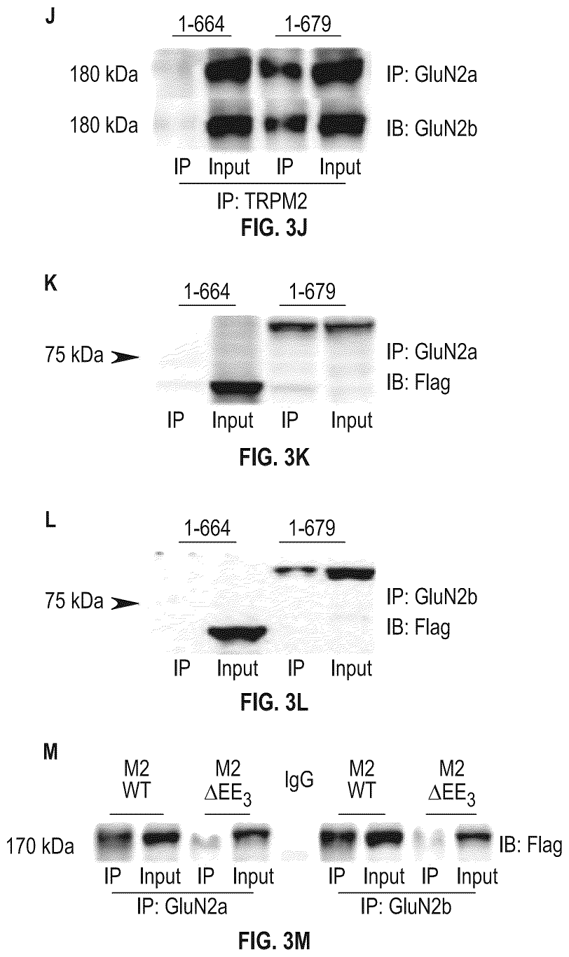

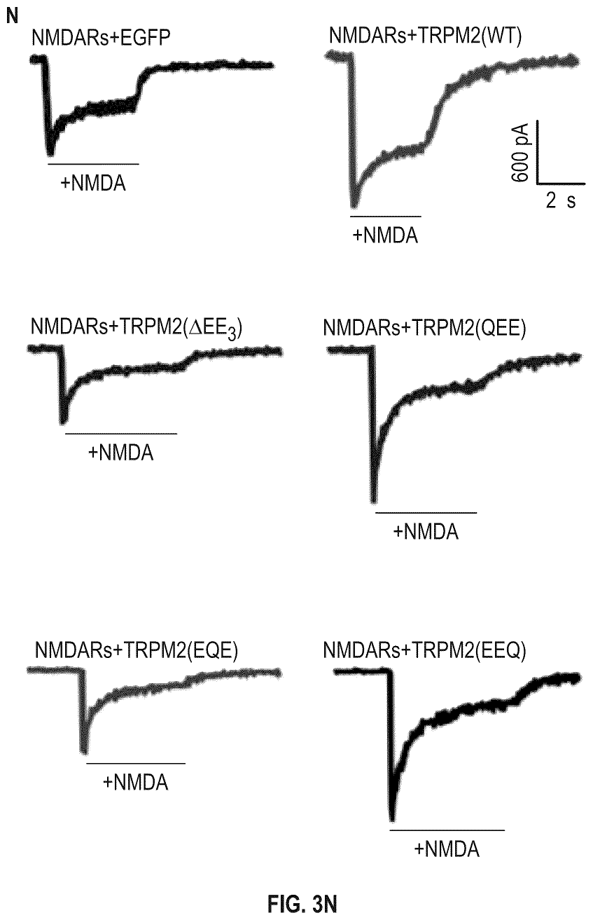

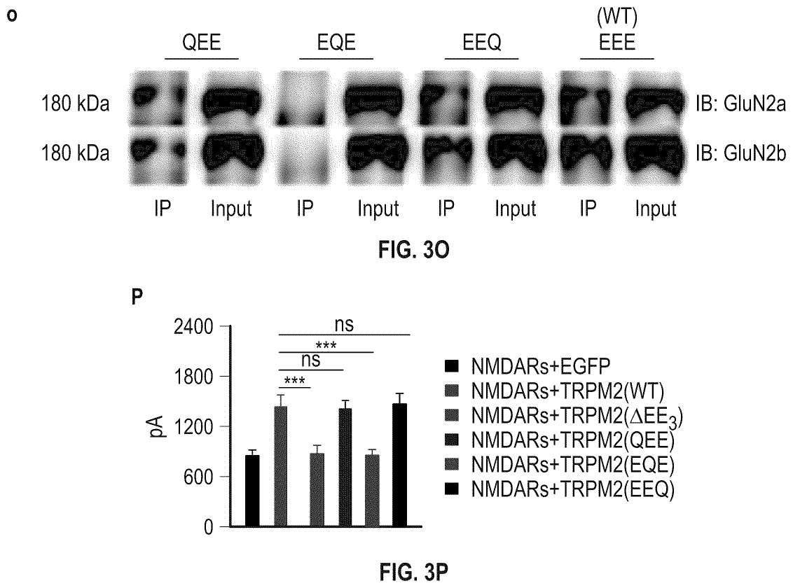

To understand how TRPM2 interacts with NMDARs, N- and C-terminus fragments of TRPM2 tagged with Flag and GFP, respectively (Flag-TRPM2-NT, GFP-TRPM2-CT) were generated. When co-expressed with NMDARs, the TRPM2-N but not the TRPM2-C fragment was detected in the precipitate pulled down by GluN1, GluN2a and GluN2b antibodies (FIGS. 3A and 3B). For NMDARs, the C-termini of GluN2a and GluN2b were pulled down by TRPM2, whereas the C-terminal domain-deleted GluN2a (GluN2a-ΔCT) and GluN2b (GluN2b-ΔCT) were absent in the precipitates pulled down by anti-TRPM2 (FIG. 3C). These findings suggest that the C-terminal tail of NMDARs interact with TRPM2's N-terminal domain.

To determine whether the TRPM2-NT fragment is sufficient to cause functional coupling with NMDARs, NMDARs were co-transfected with the full length of TRPM2 (TRPM2-FL), TRPM2-NT, or TRPM2-CT fragments. NMDAR currents recorded in NMDARs/TRPM2-NT expressing cells were similar to those in NMDARs/TRPM2-FL expressing cells, whereas the currents in NMDARs/TRPM2-CT group were indifferent from those recorded in cells expressing NMDARs alone (FIGS. 3D and 3E), indicating that TRPM2-NT couples with NMDARs.

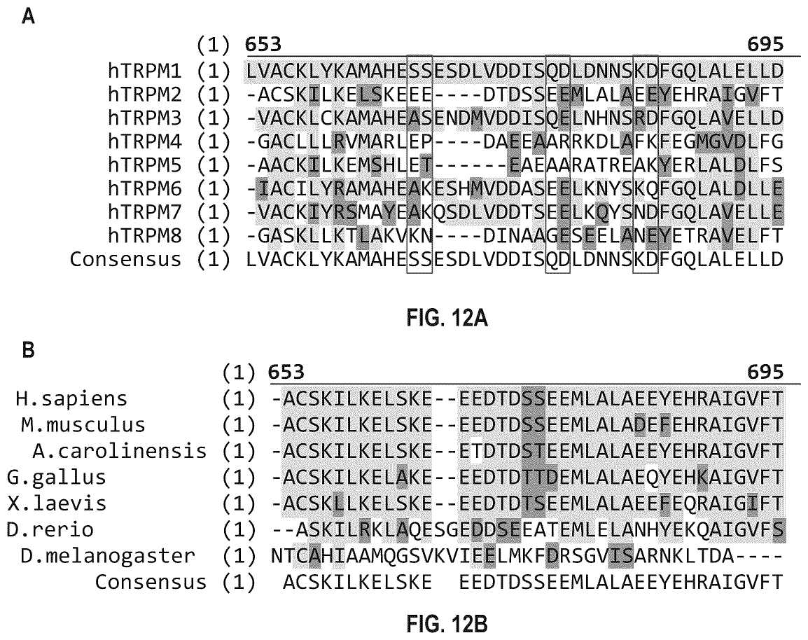

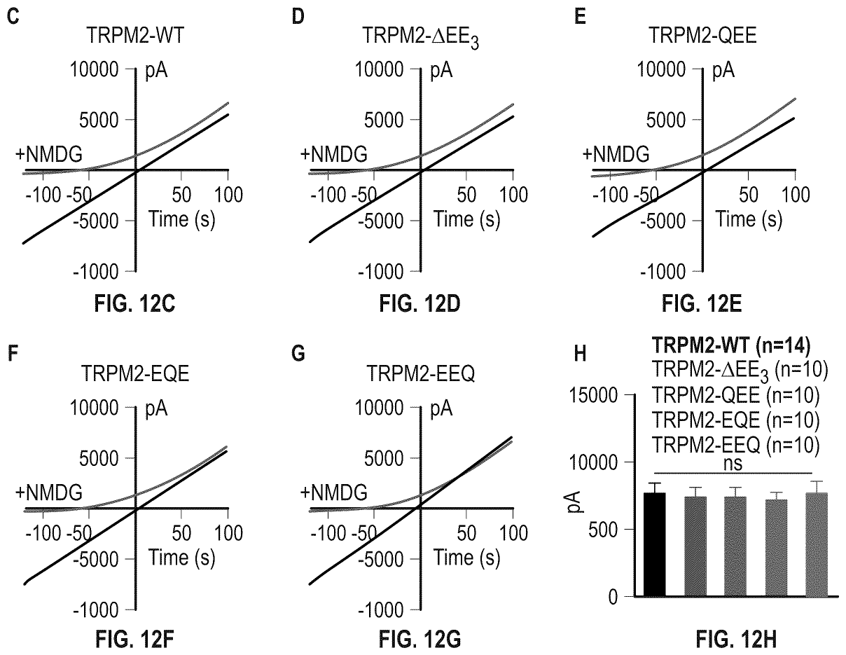

To further narrow down the NMDAR-interacting domain at the N-terminus of TRPM2 (FIG. 3F), a series of N-terminal truncation constructs were generated by incrementally deleting about 50 residues, and tested which fragments interact with NMDARs (FIGS. 3G-3I). It was found that the N-terminal amino acid residues from 631 to 679 are critical for the TRPM2 interaction with NMDARs (FIGS. 3G-3I), as both forward co-IP and reverse co-IP confirmed the interaction for the fragments of 1-727 and 1-679, whereas the fragments shorter than 631 residues failed to interact with NMDARs. Further deletion of the fragment 1-679 showed that the amino acid resides between 665 and 679 are essential for the interaction of TRPM2 and NMDARs (FIGS. 3J-3L). Interestingly, the 15 residues between 665 and 679 contains two “glutamate-glutamate” (EE) repeats separated by five residues and followed by another EE repeat (FIG. 3F). Accordingly, the residues between 665 to 681 were named the “EE3” domain for simplicity. This EE3 domain is present in TRPM2 from different species, but not present in other TRPM channels (FIGS. 12A-B). When the EE3 domain was deleted from the full-length TRPM2 (TRPM2-ΔEE3), interaction between TRPM2 and NMDARs was completely disrupted (FIG. 3M). Intriguingly, when the middle “EE” of the EE3 domain was replaced by “QQ” (glutamine), the TRPM2-EQE mutant failed to interact with NMDARs, whereas mutations of the first and third EE repeats (TRPM2-QEE, TRPM2-EEQ) did not influence TRPM2 and NMDARs interaction (FIG. 3O). These results indicate that the EE residues in the middle of the EE3 domain are critical for the TRPM2-NMDR interaction.

To investigate the functional consequence of disrupting physical interaction between TRPM2 and NMDARs, wild-type TRPM2 or TRPM2 EE3 mutants were co-expressed with NMDARs in HEK293 cells for current recording. Consistent with the disrupted interaction, TRPM2-ΔEE3 and TRPM2-EQE mutants failed to enhance NMDAR currents, whereas TRPM2-QEE and TRPM2-EEQ mutants increased NMDAR currents, similar to the potentiation of NMDAR currents induced by WT-TRPM2 when co-expressed with NMDARs in HEK-293T cells (FIGS. 3N and 3P). It is noteworthy that the TRPM2 mutations did not affect TRPM2 channel function (FIGS. 12C-12H). These results indicate that the EE3 domain is essential for physical and functional coupling between TRPM2 and NMDARs.

Identification of the TRPM2-interacting domain KKR at the C-terminus of NMDAR.

The C-tails of NMDAR subunits, GluN2a and GluN2b, have diverged substantially in evolution. As the EE3 motif in TRPM2 is largely negatively charged (FIG. 12A and FIG. 12B), a segment of GluN2a/GluN2b regions (GluN2a: 1254-1270; GluN2b: 1259-1274; FIG. 15C) with positive charges by lysine (K) and arginine (R), which is near the binding site for CaMKII. This region was designed as the “KKR” motif (FIG. 16A). The KKR motif is highly conserved between GluN2a and GluN2b and in different species (FIG. 15B and FIG. 15C). Deletion of KKR motif abolished the interaction etween TRPM2 and GluN2a/GluN2b (FIG. 15A and FIG. 15B), indicating that KKR motif is required for the TRPM2-NMDAR interaction.

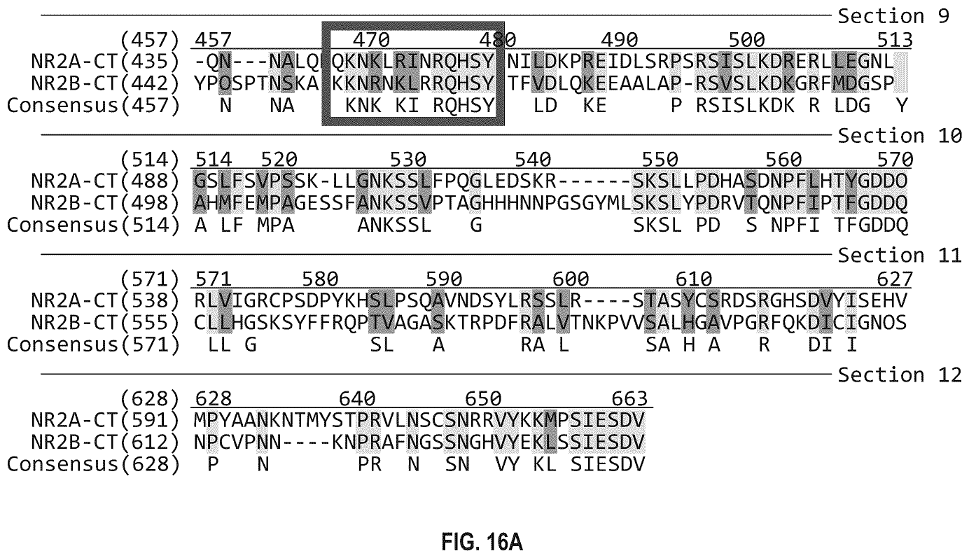

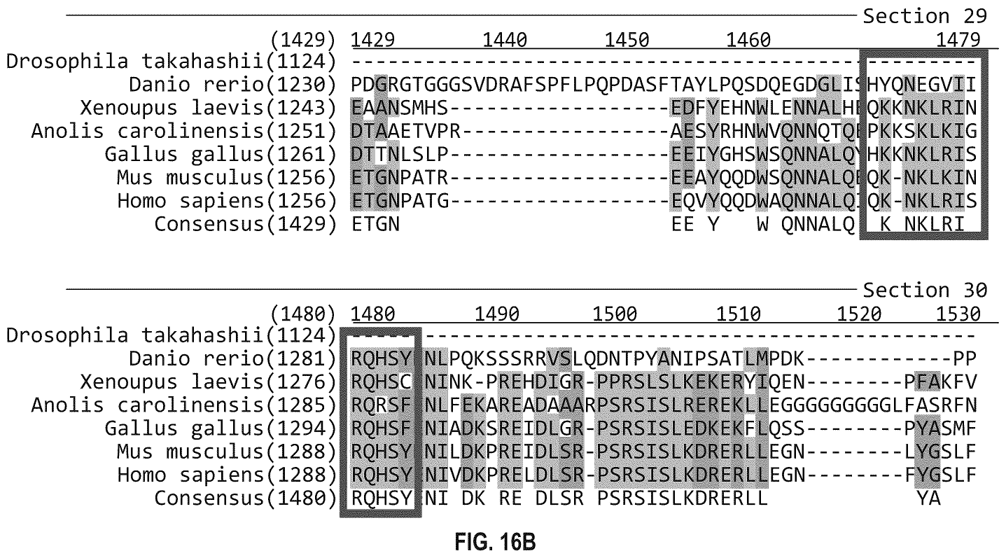

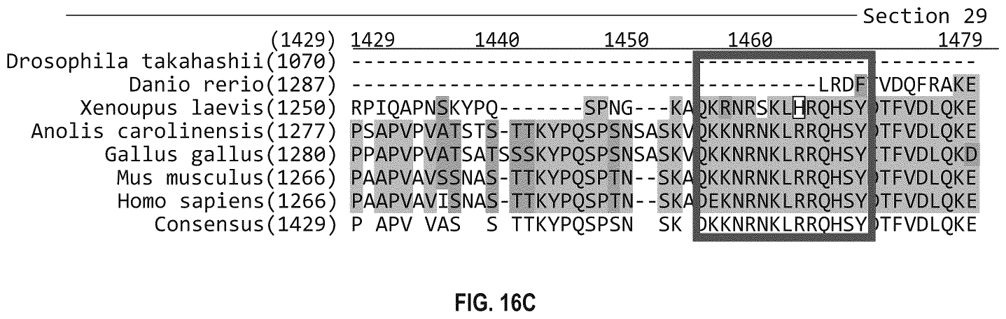

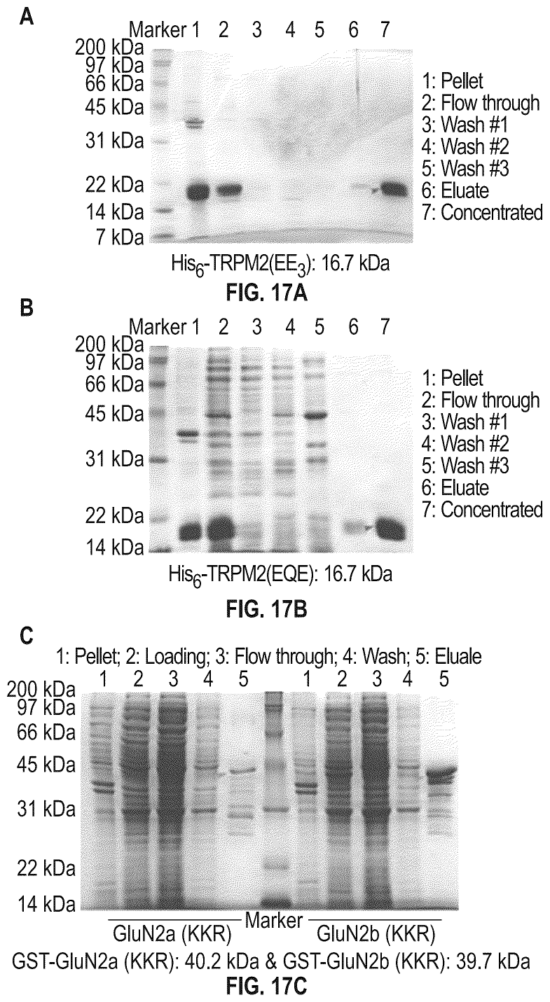

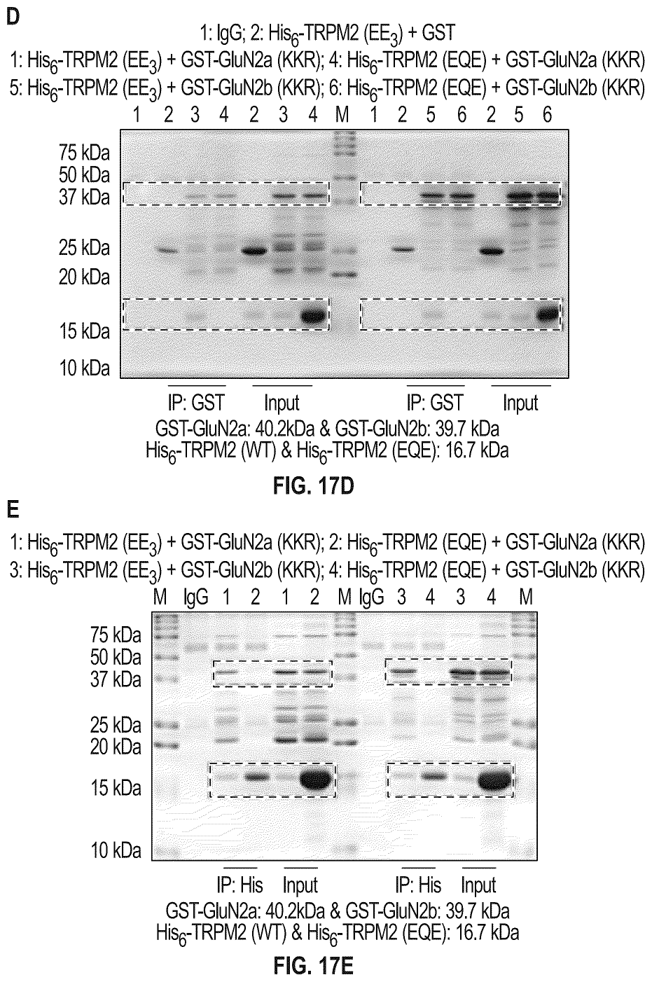

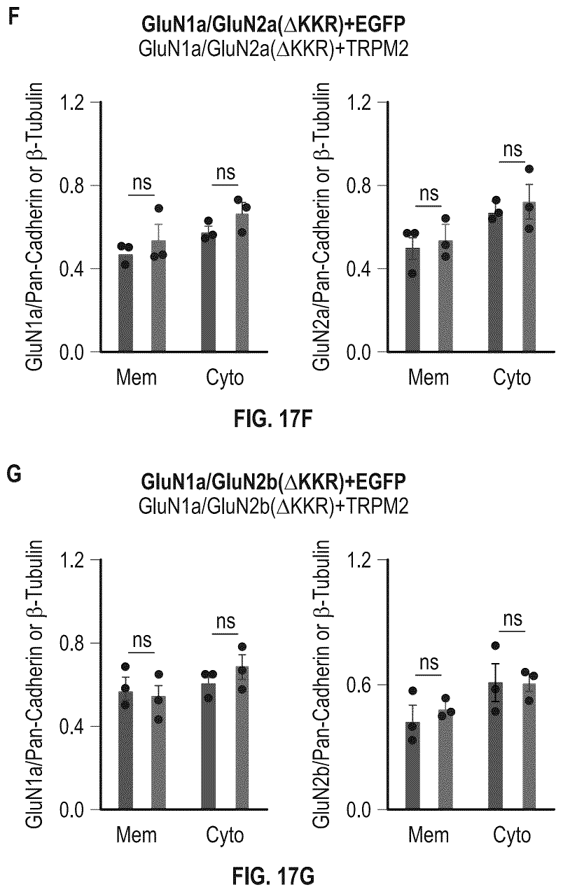

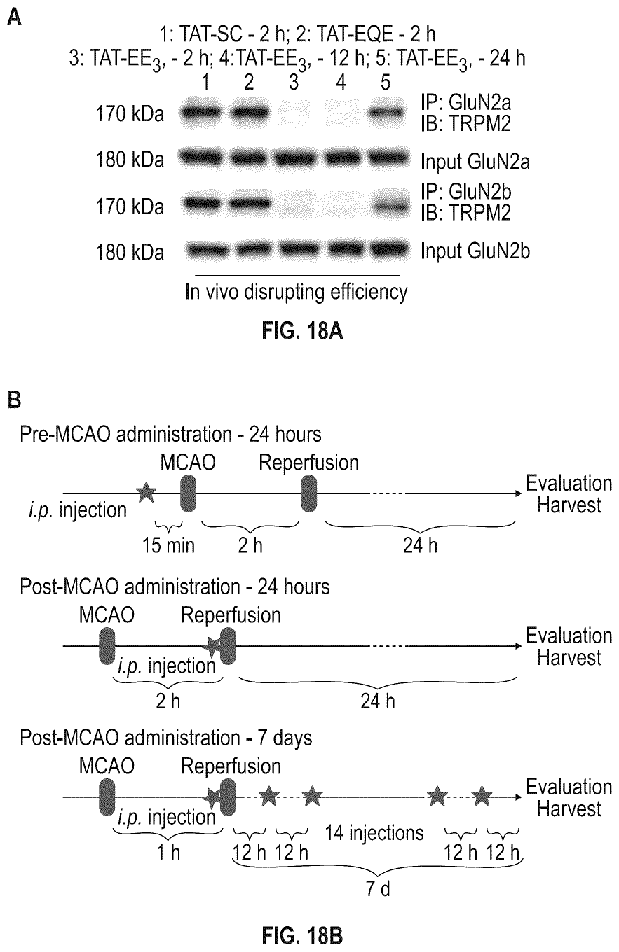

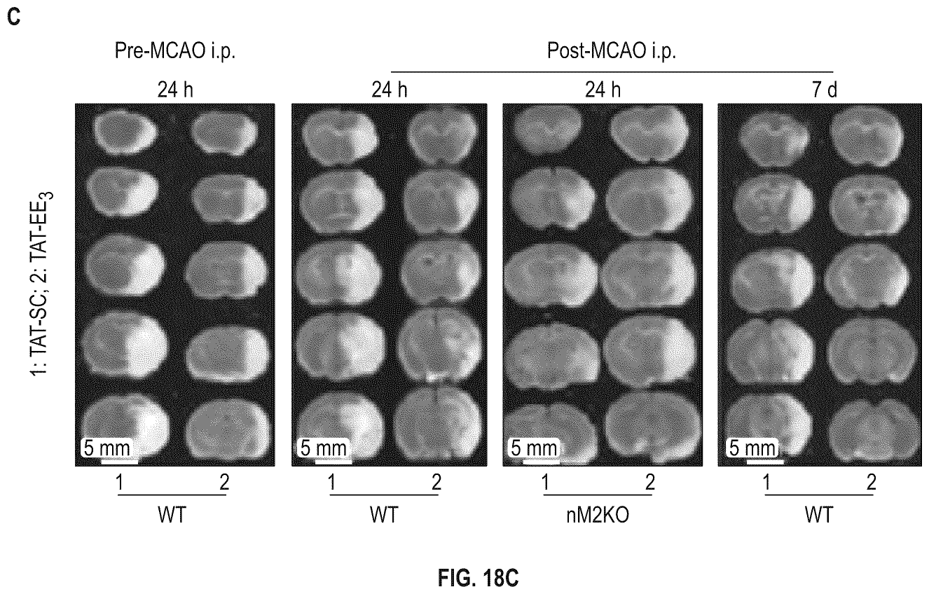

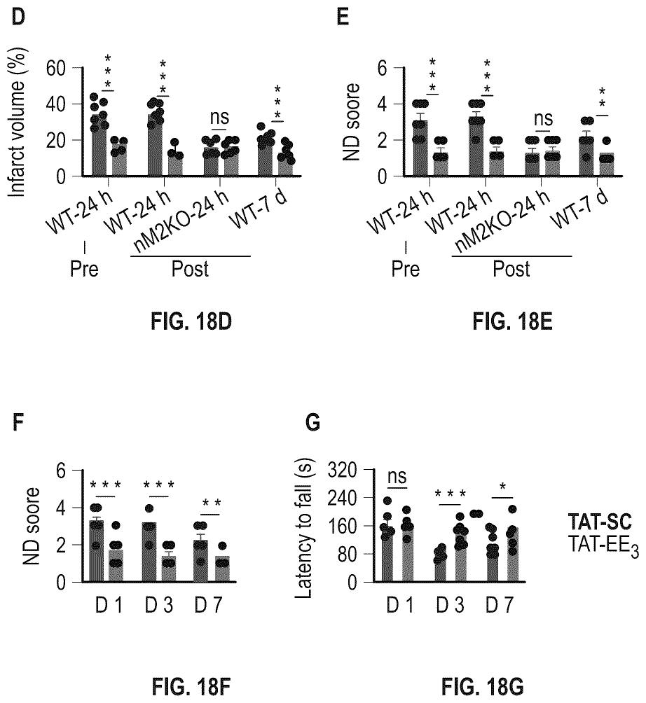

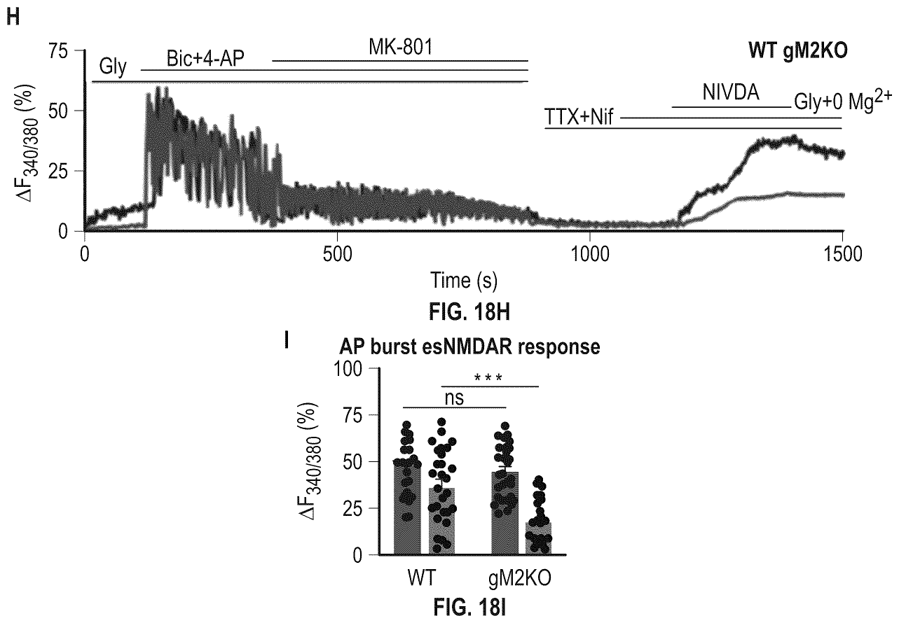

Next, it was determined whether EE3 and KK4 motifs directly bind to each other. A 117-residue TRPM2 segment containing either the EE3 or EQE motif was subcloned into a modified His6-tag (SEQ ID NO: 10) vector (FIG. 15D, FIG. 17A, and FIG. 17B), and the KKR-containing fragments in GluN2a (110 residues) and GluN2b (111 residues) were subcloned into a modified GST-tag vector for expression and purification (FIG. 17C). In vitro coIP binding assays were performed (FIG. 15D). EE3 motif was coimmunoprecipitated by KKR motifs derived from GluN2a/2b, but EQE was not (FIGS. 15E, 15F and 17D). Similarly, KKR motifs were effectively coimmnoprecipitated by EE3 motif, but not EQEQ MOTIF (FIG. 17E). These results indicate that TRPM2-GluN2a/GluN2b association is mediated by direct interaction between their EE3 and KKR motifs, respectively. Moreover, deletion of KKR motif abolished the increased surface expression of NMDAR (FIGS. 15G, 15H, 17F and 17G) and the enhanced NMDAR currents (FIGS. 151 and 15J) induced by TRPM2, confirming the importance of KKR motif in the functional coupling of TRPM2-NMDARs.

### Mechanisms of TRPM2 and NMDARs Functional Coupling

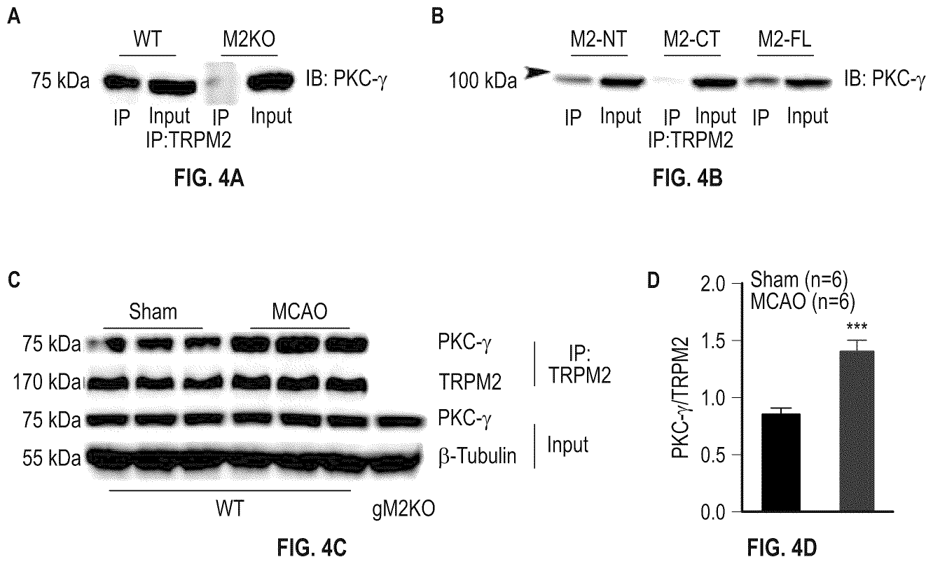

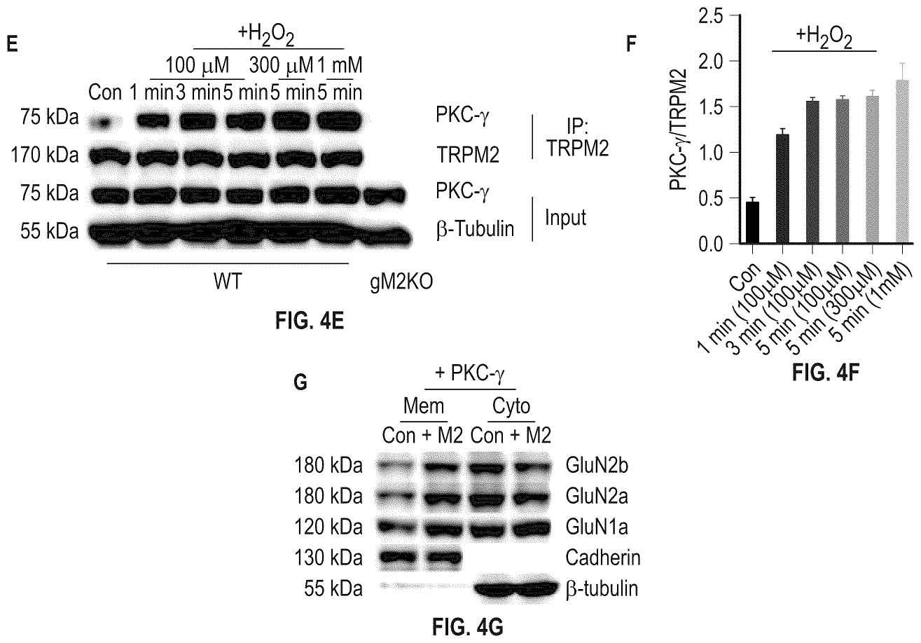

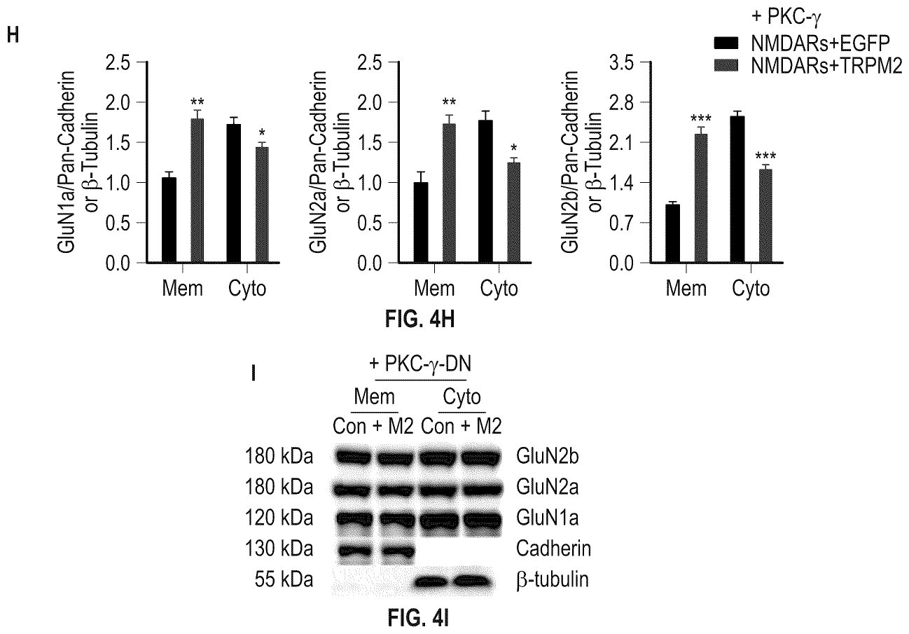

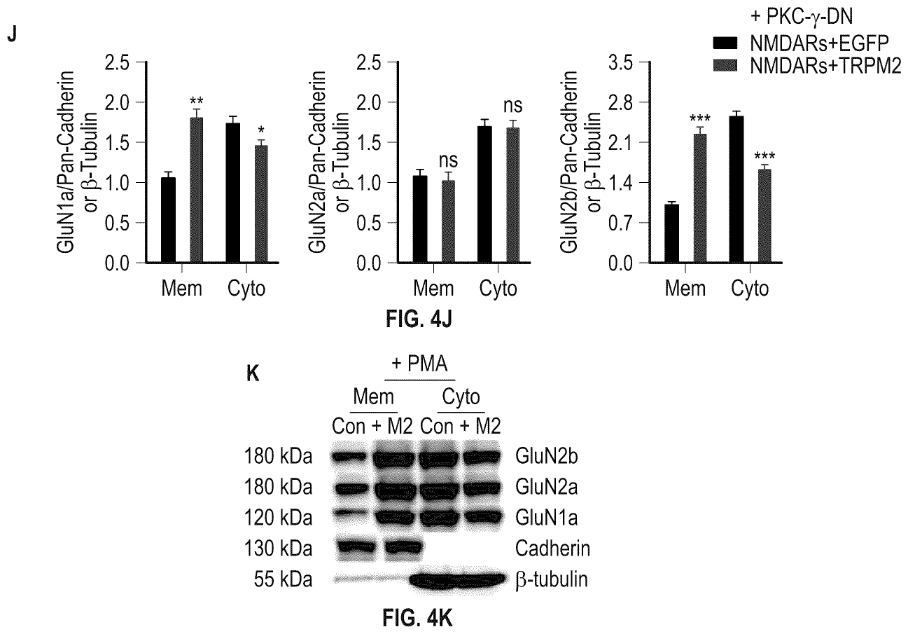

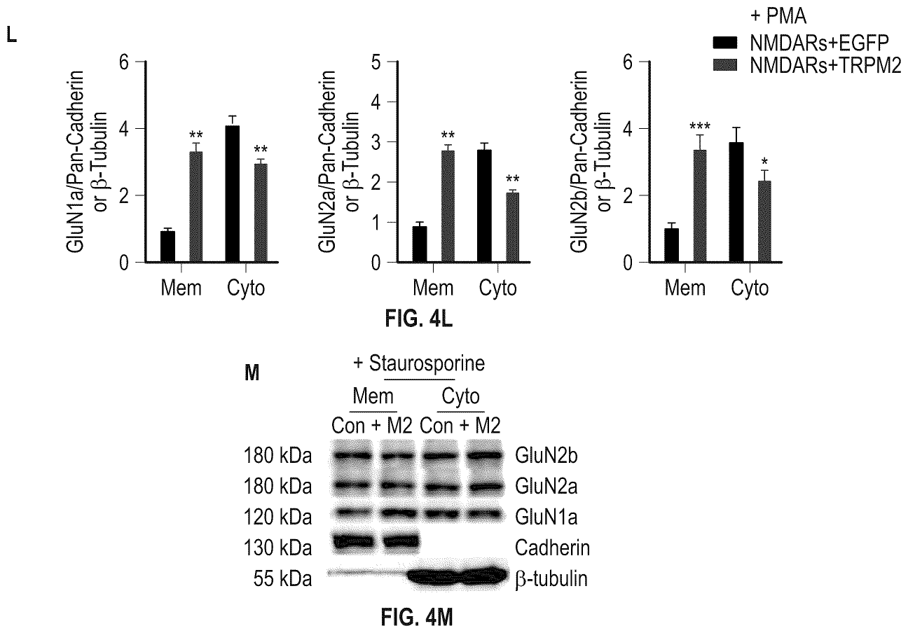

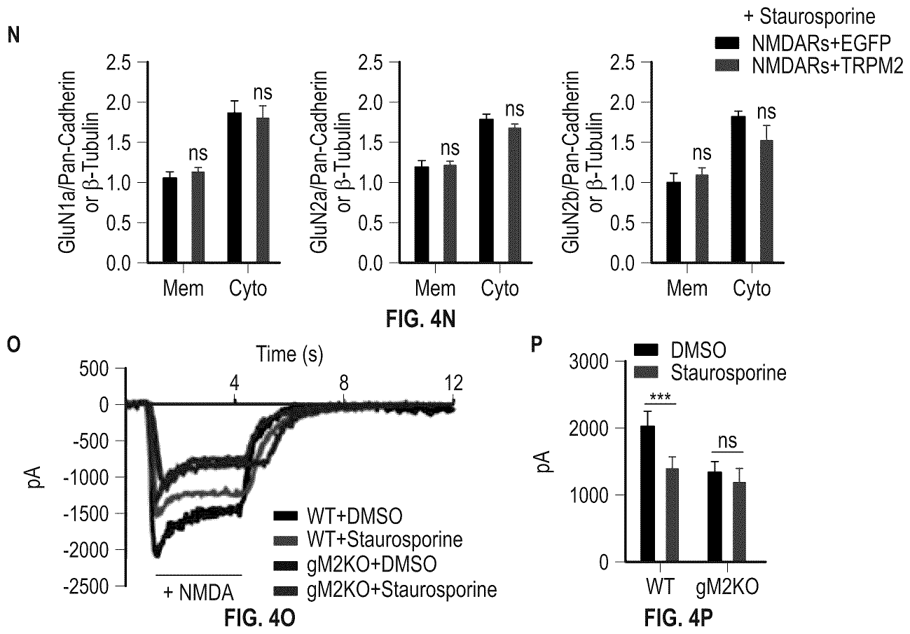

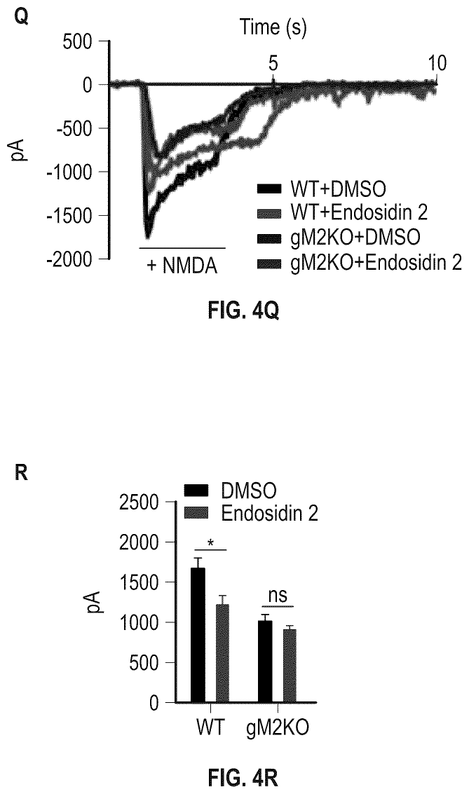

The above results indicate that TRPM2 interacts with NMDARs through the TRPM2-EE3 domain thereby enhancing NMDARs function via increasing surface localization of NMDARs (FIGS. 2A-2L and 3A-3P). Next, it was asked how the interaction of TRPM2 with NMDARs enhances surface expression of NMDARs. Since it has been previously shown that PKC regulates NMDARs trafficking to cell surface (Lan, J. Y., Skeberdis, V. A., Jover, T., Grooms, S. Y., Lin, Y., Araneda, R. C., Zheng, X., Bennett, M. V., and Zukin, R. S. (2001). Protein kinase C modulates NMDA receptor trafficking and gating. Nature neuroscience 4, 382-90; Zheng, X., Zhang, L., Wang, A. P., Bennett, M. V., and Zukin, R. S. (1999). Protein kinase C potentiation of N-methyl-D-aspartate receptor activity is not mediated by phosphorylation of N-methyl-D-aspartate receptor subunits. Proceedings of the National Academy of Sciences of the United States of America 96, 15262-67.), it was reasoned that PKC could be part of NMDARs-TRPM2 complex. Thus, whether PKC interacts with TRPM2 was investigated. Using WT and global TRPM2-KO (gM2KO) mouse brains after MCAO, we found that neuron-specific PKC can be readily pulled down by anti-TRPM2 in WT but not in TRPM2-KO (gM2KO) brains (FIG. 4A). In HEK293 cells over-expressing PKCγ with full-length TRPM2 (TRPM2-FL), TRPM2-CT or TRPM2-NT, we found that TRPM2-FL and TRPM2-NT, but not TRPM2-CT, pulled down PKCγ, indicating that PKCγ interacts with TRPM2-NT (FIG. 4B). Moreover, when brain tissues from both sham and MCAO mice were used, the amount of PKCγ pulled down by anti-TRPM2 antibody in MCAO mice was about 1.5-fold of that from sham mice (FIGS. 4C and 4D), suggesting that oxidative stress during MCAO increased TRPM2 and PKCγ interaction. Indeed, cultured neurons treated with H2O2 exhibited significantly increased TRPM2 and PKCγ interaction (FIGS. 4E and 4F).

To investigate whether PKCγ influences surface expression levels of NMDARs, NMDARs and TRPM2 were co-expressed with wild type PKCγ or dominant-negative PKCγ (PKCγ-DN). As shown in FIGS. 4G-4J, over-expression of PKCγ further increased surface expression of NMDARs, whereas dominate negative PKCγ abolished the increase of surface NMDAs by TRPM2, suggesting that a functional PKCγ is required for increased NMDARs trafficking induced by TRPM2.

Pharmacological tools were also used to probe the function of PKC in the TRPM2-mediatd enhancement of surface NMDAR levels. HEK293T cells transfected with NMDARs and TRPM2 exhibited much higher level of surface NMDARs after treatment with PKC activator PMA (FIGS. 4K-4L), whereas PKC inhibitor stausporine abolished the enhanced surface expression level of NMDARs (FIGS. 4M-4N). The effects of PKC on NMDARs were further confirmed by the effects of PKC inhibitor stausporine on NMDAR currents recorded in the neurons from WT mice. Stausporine normalized NMDAR current amplitude in the WT neuron to the similar amplitude level of NMDAR current in TRPM2-KO neurons (FIGS. 4O-4P).

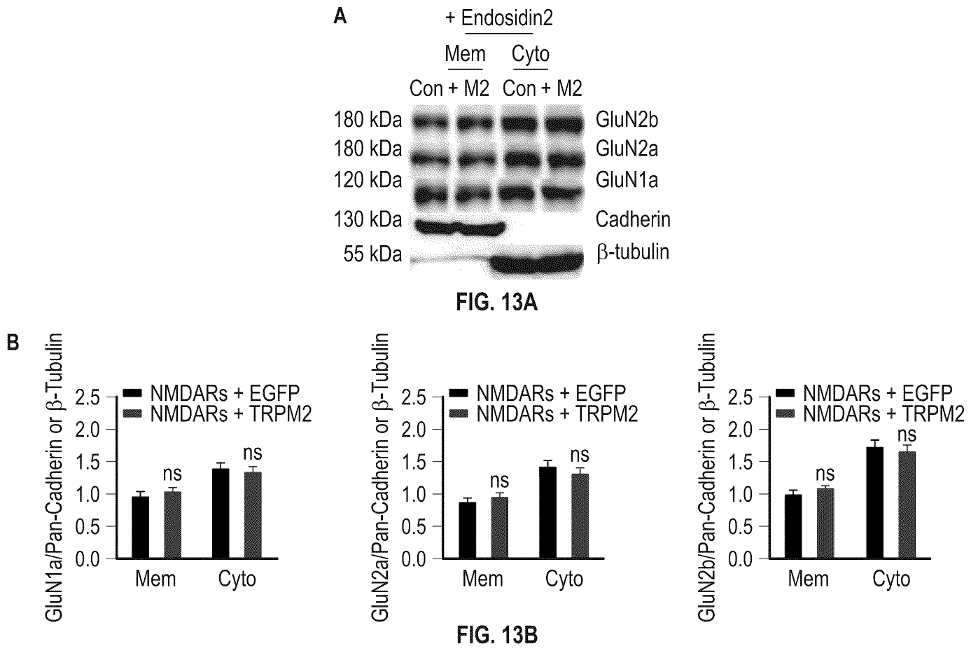

As trafficking of NMDARs to plasma membrane involves exocyst (Sans, N., Prybylowski, K., Petralia, R. S., Chang, K., Wang, Y. X., Racca, C., Vicini, S., and Wenthold, R. J. (2003). NMDA receptor trafficking through an interaction between PDZ proteins and the exocyst complex. Nature cell biology 5, 520-30), the effects of blocking exocyst on NMDARs surface expression were tested. Endosidin 2, an inhibitor of exocysts, prevented the increase of surface expression level of NMDARs (FIGS. 13A-13B) induced by co-expression with TRPM2, and abolished the increased current amplitude of NMDARs by TRPM2 in WT neurons (FIGS. 4Q-4R).

Taken together, the above results indicate that PKCγ interacts with TRPM2, which can be promoted by oxidative stress condition and by MCAO in vivo, and that interaction of PKCγ with TRPM2 enhances NMDARs surface trafficking likely via exocysts.

### Disrupting Peptide Eliminates Physical Interaction and Functional Coupling of TRPM2 and NMDARs

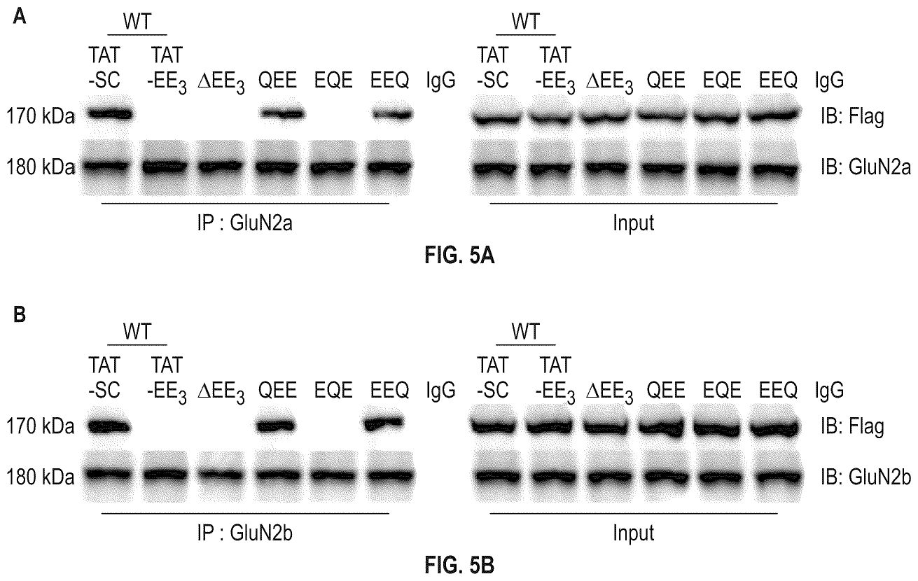

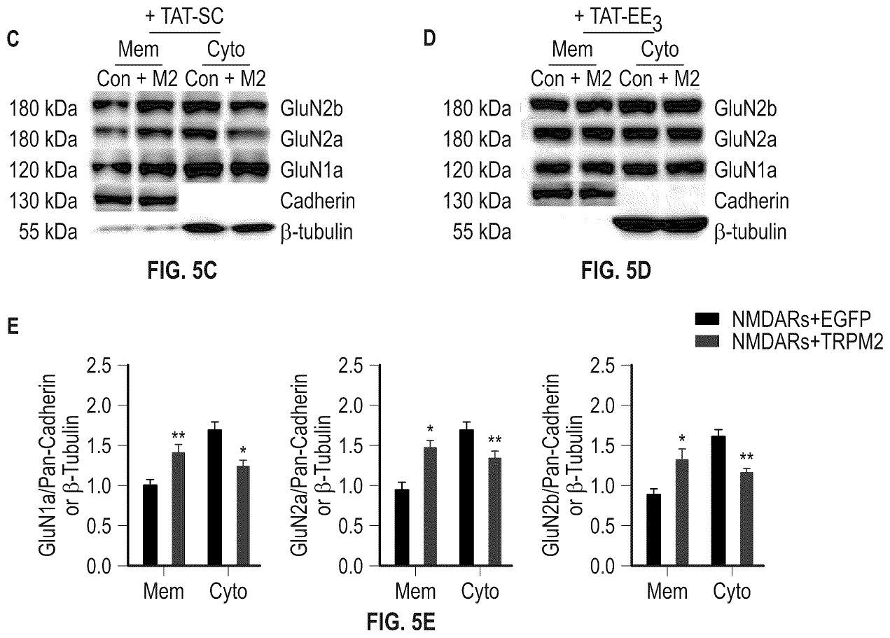

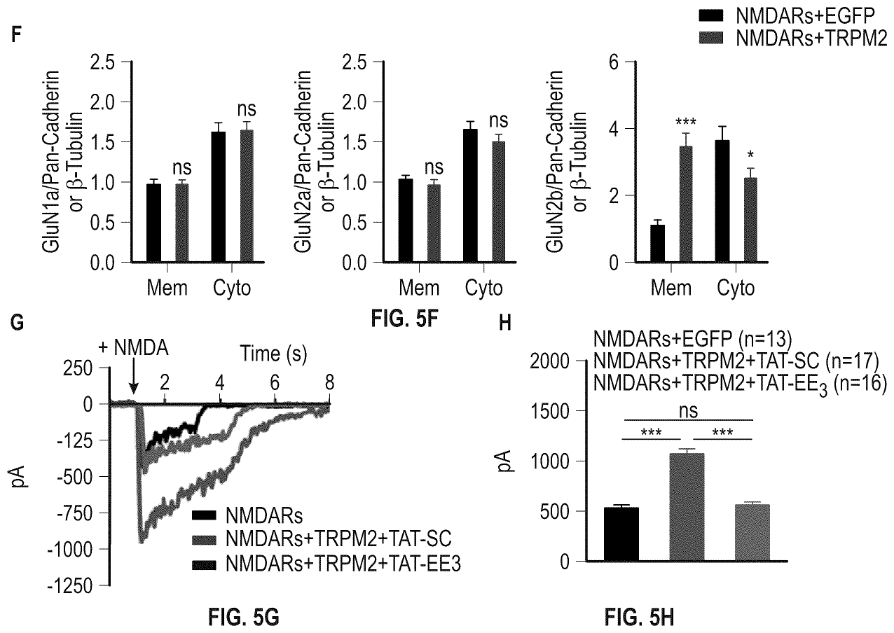

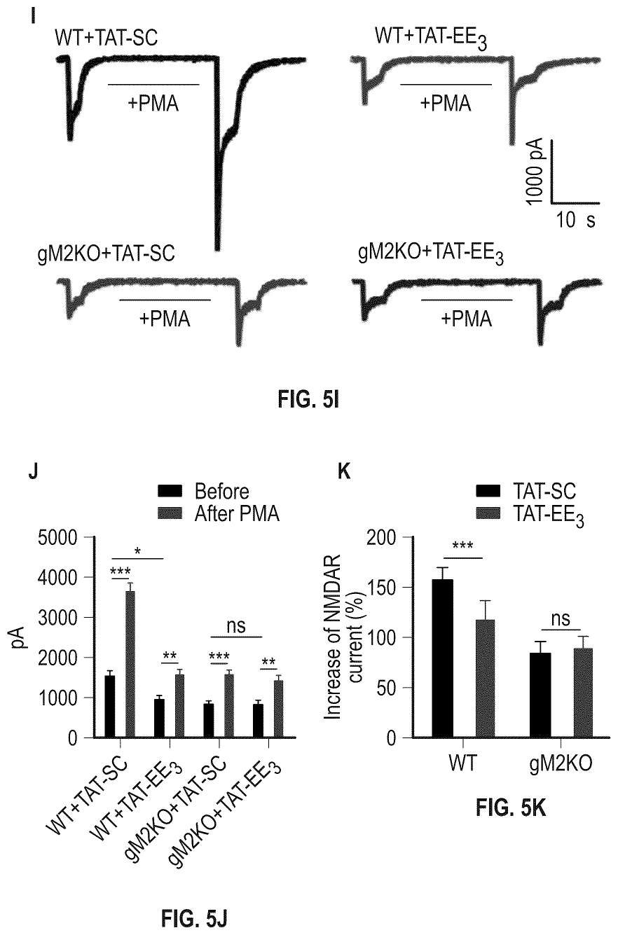

As the TRPM2 N-terminal EE3 domain is critical for TRPM2 and NMDARs interaction, we designed membrane-permeable peptides, TAT-EE3 and scrambled control peptide TAT-SC, to investigate whether disruption of physical interaction eliminates the functional coupling between TRPM2 and NMDARs. To determine whether TAT-EE3 is able to disrupt the interaction of TRPM2 and NMDARs, co-IP experiments using NMDARs co-expressed with WT-TRPM2 and TRPM2 EE3 mutants including TRPM2-QEE, TRPM2-EQE, TRPM2-EEQ, and the deletion mutant TRPM2-ΔEE3 were conducted. Similar to the TRPM2-A (EE) 3 and TRPM2-EQE mutants, TAT-EE3 treatment completely disrupted the interaction of TRPM2 with GluN2a (FIG. 5A) and GluN2b (FIG. 5B), whereas TAC-SC treatment and mutants TRPM2-QEE and TRPM2-EEQ did not influence TRPM2 and GluN2a and GluN2b interactions. We further determined the surface expression of NMDARs after TRPM2 and NMDARs over-expressing cells were treated with TAT-EE3 or TAT-SC. TAT-EE3 eliminated the enhancement of NMDAR surface expression by TRPM2 (FIGS. 5D and 5F), whereas TAT-SC exhibited no influence (FIGS. 5C and 5E). Consistent with the disrupted interaction between TRPM2 and NMDARs and the eliminated increase of NMDAR surface expression, the NMDAR current amplitude in NMDARs and TRPM2 overexpressing cells treated with TAT-EE3 was significantly smaller than that in cells treated with TAT-SC (FIGS. 5G and 5H).

As PKC activation can both increase NMDARs trafficking to cell surface and regulate channel activity (Lan, J. Y., Skeberdis, V. A., Jover, T., Grooms, S. Y., Lin, Y., Araneda, R. C., Zheng, X., Bennett, M. V., and Zukin, R. S. (2001). Protein kinase C modulates NMDA receptor trafficking and gating. Nature neuroscience 4, 382-90), it was sought to determine the PMA effects on NMDAR currents in neurons treated with TAT-EE3 or TAT-SC. Cultured neurons from WT and TRPM2-KO mice were treated with TAT-EE3 or TAT-SC overnight. NMDAR currents were elicited before and after 20 s perfusion with PMA. As shown in FIG. 5I (top left), NMDA-induced current was increased from 1542.1±117.1 pA to 3642.1±180.8 pA (FIG. 5J) by PMA perfusion for 20 s (FIGS. 5J-5K), about 1.5-fold increase, in neurons from WT mice. However, in the neurons incubated with TAT-EE3, the NMDAR current amplitude was much smaller before and after PMA perfusion (FIG. 5I, top right and FIG. 5J), and the increase of current amplitude induced by PMA was also significantly smaller than that in neurons incubated with TAT-SC (FIG. 5K). The smaller NMDAR currents before PMA and smaller increase of NMDAR currents after PMA in TAT-EE3 incubated neurons suggest that disruption of TRPM2 and NMDARs interaction may have affected surface trafficking of NMDARs induced PKC activation. In the neurons from global TRPM2-KO (gM2KO) mice, the NMDAR current amplitude (FIG. 5I, bottom) and PMA induced increase in NMDAR currents were much smaller than that of WT neurons. Moreover, there was no difference in PMA-induced changes of NMDAR currents between TAT-SC and TAT-EE3 incubated neurons from TRPM2-KO mice (FIG. 5J-5K). These results indicate that similar to TRPM2-KO, disruption of the interaction between TRPM2 and NMDARs in the WT neuron by TAT-EE3 reduced NMDAR currents, and attenuated PMA induced increase in NMDAR currents. As PMA causes an increase of NMDAR channel activities and surface trafficking, it is conceivable that in TRPM2-KO and TAT-EE3 treated neurons, PMA failed to cause surface trafficking of NMDARs. This notion is further supported by the fact that inhibition of exocysts abolished the increase of surface expression of NMDARs co-expressed with TRPM2 in HEK293T cells (FIGS. 13A-13B), and the enhancement of NMDAR currents by TRPM2 in WT neurons (FIG. 4Q-4R).

### Disruption of TRPM2 and NMDAR Interaction Protects Neurons Against Ischemic Injury In Vitro

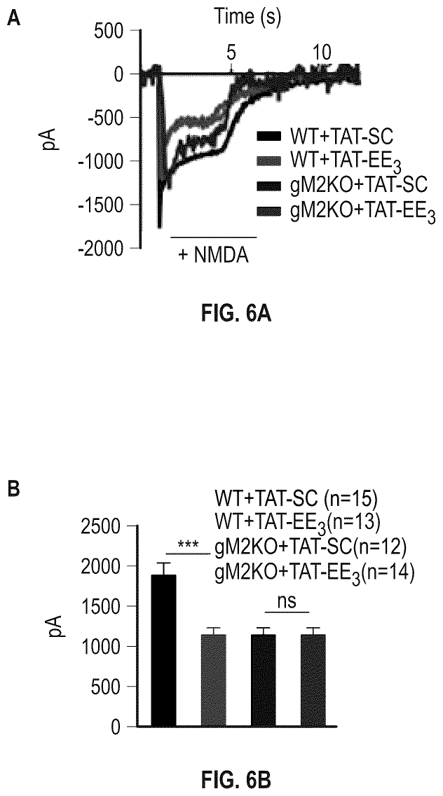

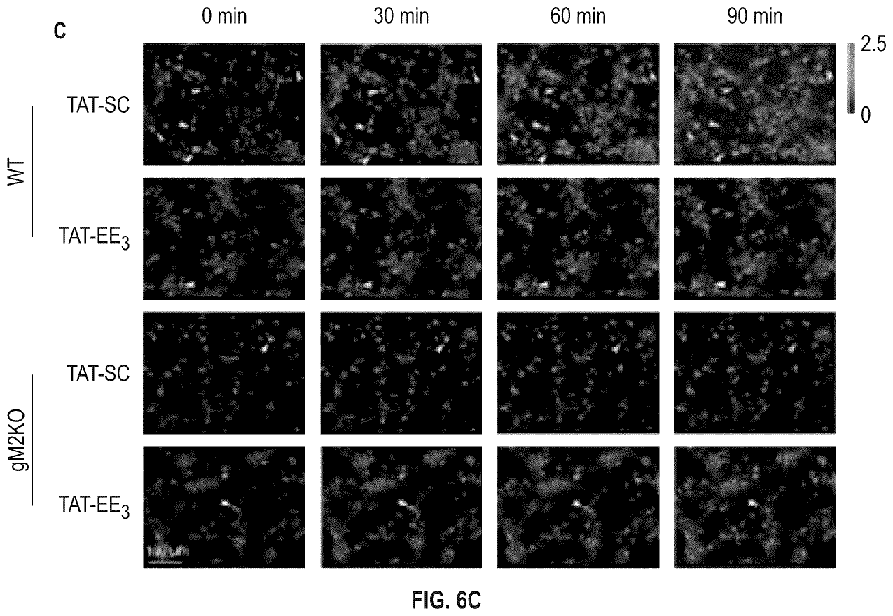

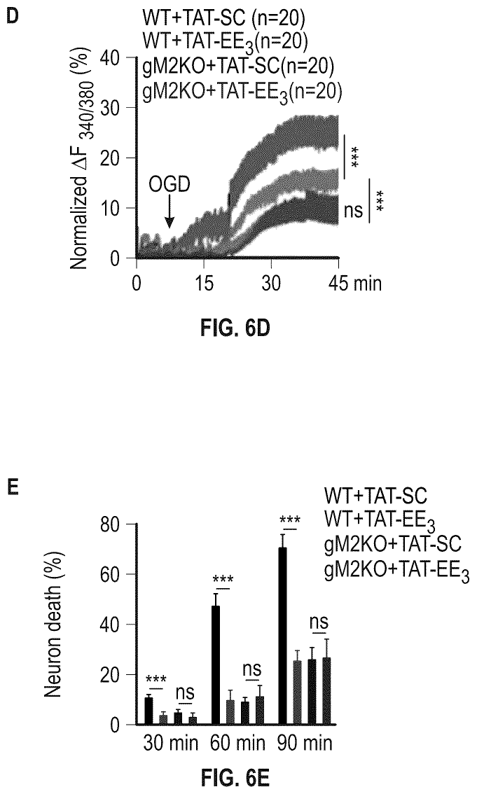

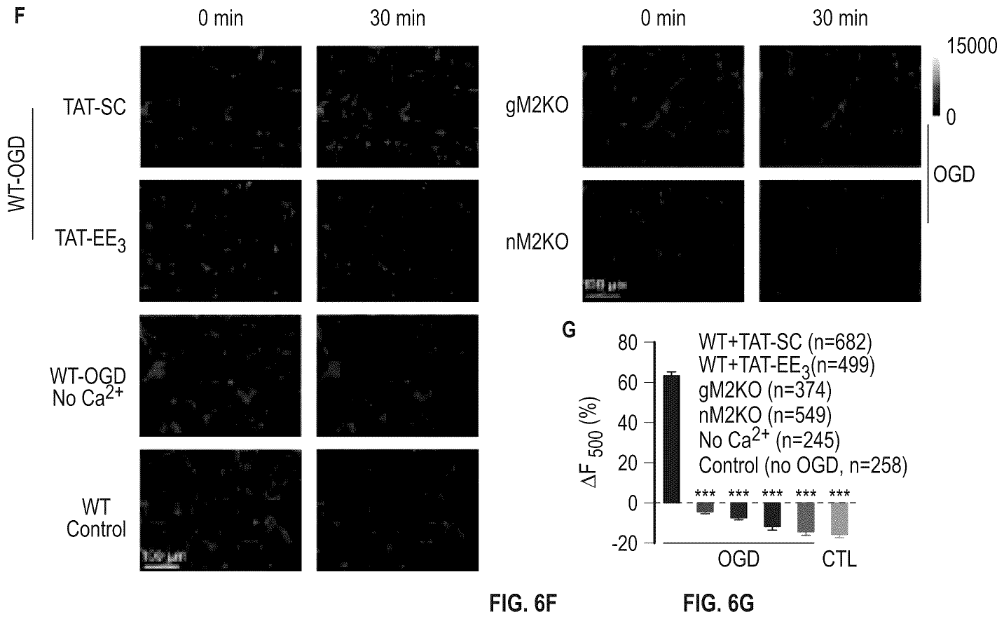

The functional uncoupling of TRPM2 from NMDARs by TAT-EE3 promoted us to investigate its significance during ischemic injury. Using cultured neurons, the disrupting peptide TAT-EE3 efficiently reduced NMDAR currents in neurons from WT mice but not from TRPM2-KO mice, whereas TAT-SC exhibited no influence on NMDAR currents from WT and TRPM2-KO neurons (FIGS. 6A and 6B), indicating the specificity of TAT-EE3. To mimic in vivo ischemia condition, OGD condition to treat cultured neurons was used. When neurons were exposed to OGD, the changes of intracellular Ca2+ were significantly reduced in WT but not in TRPM2-KO neurons by pretreatment with TAT-EE3 (FIGS. 6C and 6D). The OGD-mediated neuronal death was 10.6%, 47.6% and 70.5% at 30 min, 60 min and 90 min respectively in WT neurons, which was drastically reduced to 3.6%, 9.9% and 45.2% by TAT-EE3, similar to the percentage death rate in the global TRPM2-KO (gM2KO) neurons (FIG. 6E).

Next it was evaluated whether TAT-EE3 influences OGD-mediated mitochondrial dysfunction using the Rh123 florescence de-quenching assay (Nguyen, P. V., Marin, L., and Atwood, H. L. (1997). Synaptic physiology and mitochondrial function in crayfish tonic and phasic motor neurons. Journal of neurophysiology 78, 281-94). As shown in FIGS. 6F-6G, OGD caused mitochondria depolarization as reflected by the increase of Rh123 fluorescence in WT neurons treated with TAT-SC. In contrast, mitochondrial membrane depolarization induced by OGD was markedly inhibited in WT neurons treated with TAT-EE3, similar to the inhibited depolarization in neurons from global TRPM2-KO (gM2KO) or neuron-specific TRPM2 deletion (nM2KO) mice (FIGS. 6G-F). The mitochondria membrane depolarization was mediated by Ca2+ entry as evidenced when neurons were perfused with Ca2+ free OGD, the membrane depolarization was inhibited (FIG. 6F: micrographs at 2nd row from bottom, and FIG. 6G). These results indicate that the TAT-EE3 disrupting peptide abolishes the exacerbation of NMDARs' excitotoxicity resulted from functional coupling of TRPM2 and NMDARs and protects neurons from ischemic injury in vitro.

### Disruption of TRPM2 and NMDAR Interaction Protects Mice Against Ischemic Stroke

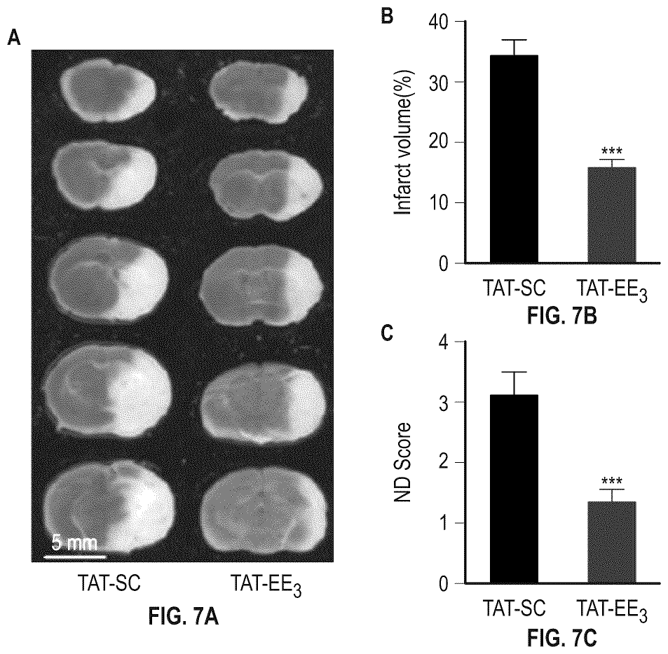

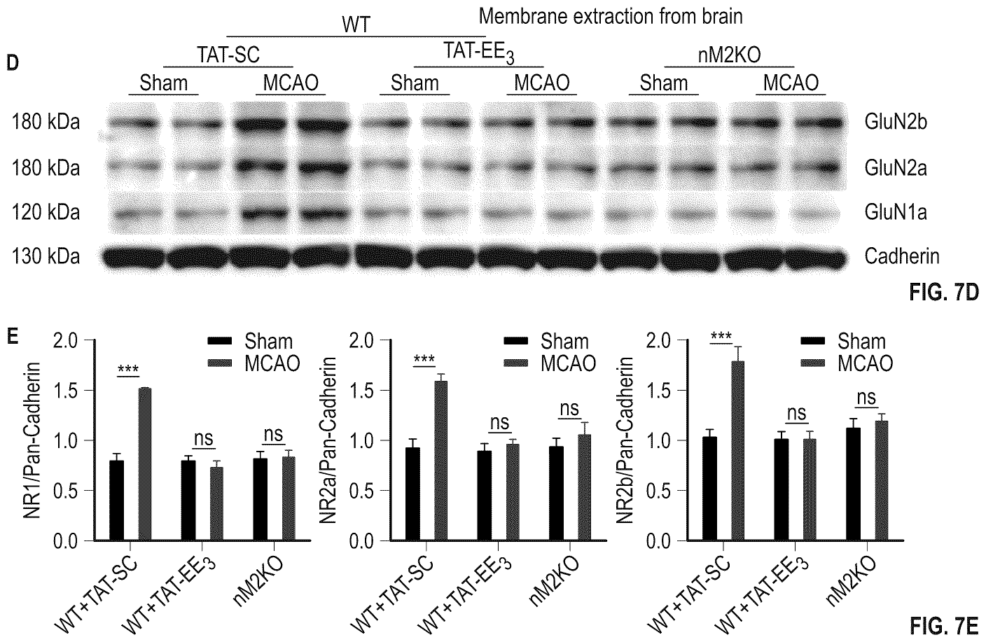

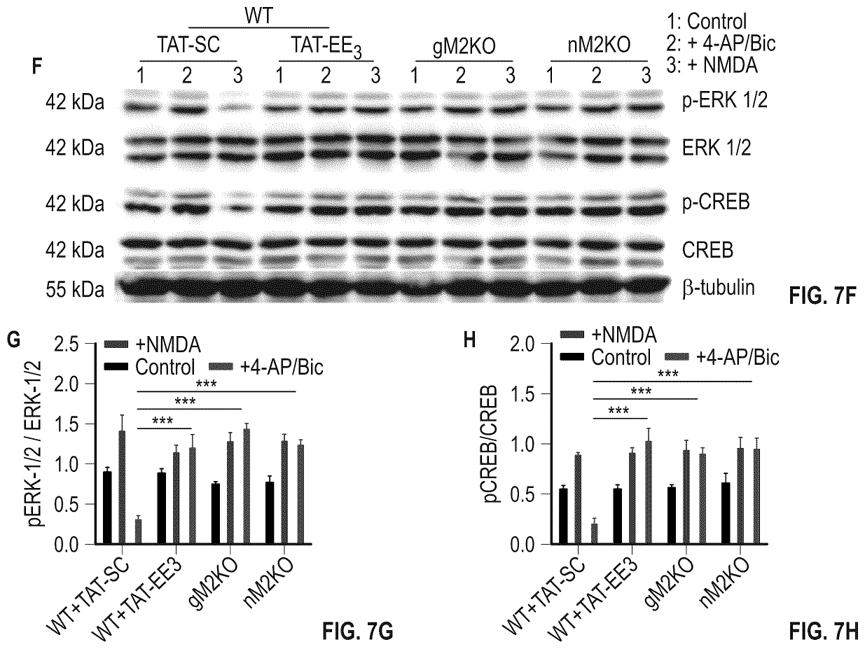

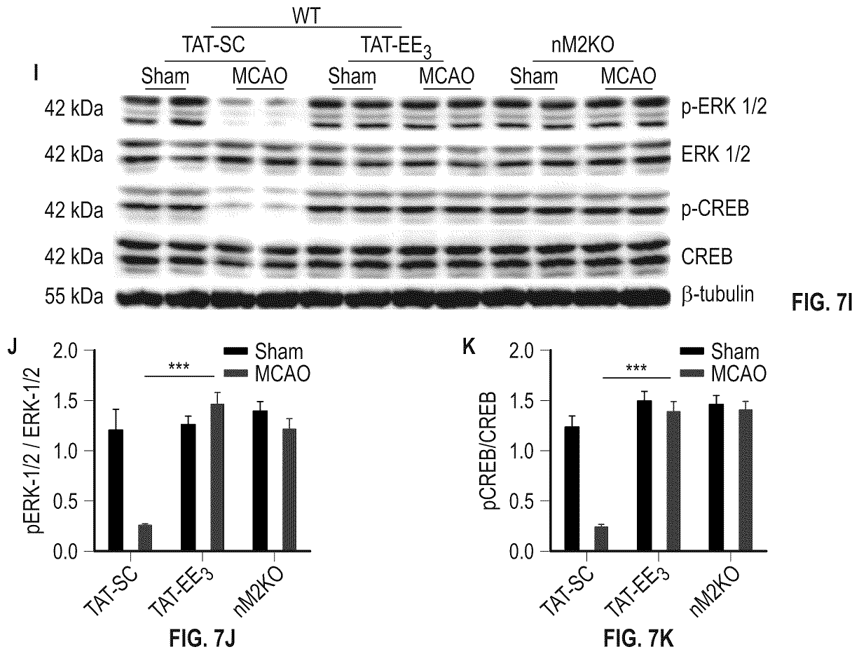

To determine if TAT-EE3 inhibits ischemic injury in vivo, TAT-EE3 or TAT-SC (100 nmol/kg) was administered 15 mins before MCAO or sham surgery as previously reported (Weilinger et al., 2016). Infarct volume and neurological deficient scores were evaluated 24 hrs after MCAO (see description for FIG. 1). As shown in FIG. 7A-7C, TAT-TEE3 treated mice exhibited significantly reduced infarct volume and improved neurological behavior scores in comparison with TAT-SC-treated mice. Like those in the global (gM2KO) (FIG. 1W) and neuronal (nM2KO) TRPM2-KO mice (FIG. 7D), the enhanced surface expression of NMDARs was abolished by TAT-EE3 (FIGS. 7D-7E) in MCAO mice, indicating that disruption of NMDARs and TRPM2 coupling is an effective approach to protect the brain against ischemia injury.

As synaptic NMDARs promote pro-survival signals whereas extrasynaptic NMDARs promote pro-death signal (Hardingham, G. E., and Bading, H. (2010). Synaptic versus extrasynaptic NMDA receptor signalling: implications for neurodegenerative disorders. Nature reviews Neuroscience 11, 682-96; Hardingham, G. E., Fukunaga, Y., and Bading, H. (2002). Extrasynaptic NMDARs oppose synaptic NMDARs by triggering CREB shut-off and cell death pathways. Nature neuroscience 5, 405-14), it was sought to determine whether the enhanced NMDAR surface expression by MCAO in WT mice is pro-survival or pro-death. Cultured neurons from global TRPM2-KO, neuronal TRPM2-KO (Cre+), or Cre-littermate WT littermates (WT) were treated for 1 h with NMDA to activate both synaptic and extra-synaptic NMDARs, or 4-AP (2.5 mM) plus 50 μM bicuculline (Bic) to activate only synaptic NMDARs (Hardingham et al., 2002; Nicolai et al., 2010). WT neurons were pre-incubated with TAT-SC or TAT-EE3 overnight. After induction of NMDAR activation by NMDA or 4-AP/Bic, neurons were harvested for quantitative analysis of CREB and ERK1/2 activation. In neurons pre-incubated with TAT-SC, treatment with 4-AP/Bic significantly increased neuronal survival signal, as indicated by the phosphorylated ERK1/2 (PERK 1/2) and phosphorylated CREK (pCREB) in comparison with control group, whereas NMDA treatment drastically reduced the pro-survival signal pERK1/2 and pCREB in WT neurons treated with TAT-SC group (FIGS. 7F-7H), similar to previous finding in neurons without any pre-incubation (Hardingham, G. E., Fukunaga, Y., and Bading, H. (2002). Extrasynaptic NMDARs oppose synaptic NMDARs by triggering CREB shut-off and cell death pathways. Nature neuroscience 5, 405-14; Nicolai, J., Burbassi, S., Rubin, J., and Meucci, O. (2010). CXCL12 inhibits expression of the NMDA receptor's NR2B subunit through a histone deacetylase-dependent pathway contributing to neuronal survival. Cell death & disease 1, e33). In stark contrast, both 4-AP/Bic and NMDA treatments increased pro-survival signal (PERK1/2 and pCREB) levels in WT neurons pre-incubated with TAT-EE3, as well as in neurons from mice with global or neuronal-specific TRPM2 deletion, indicating that disruption in the TRPM2-NMDAR interaction by TAT-EE3 or by deletion of TRPM2 largely eliminated extrasynaptic activation of NMDARs by NMDA.

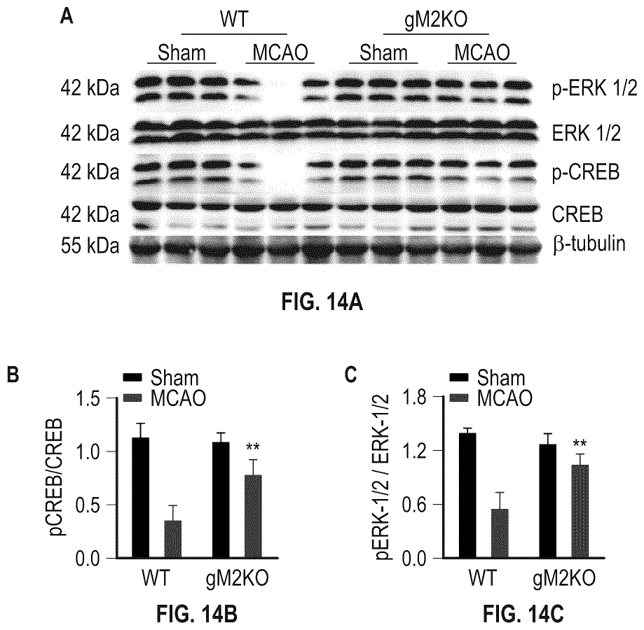

To determine whether TRPM2 exacerbates NMDARs extrasynaptic excitotoxicity in WT MCAO mice, CREB and ERK1/2 activities in the brain tissues from MCAO mice treated with TAT-SC or TAT-EE3 were further analyzed. In TAT-SC treated mice, pERK1/2 and pCREB levels were significantly lower in MCAO brains than sham control brains, whereas MCAO induced no reduction in pERK1/2 and pCREB levels in TAT-EE3 treated brains (FIGS. 7I-7K). Similarly, neuronal TRPM2-KO (FIG. 7I-K) and global TRPM2-KO (FIGS. 14A-14C) prevented reduction of pERK1/2 and pCREB levels after MCAO, indicating that in vivo disruption of TRPM2-NMDAR interaction, as well as deletion of TRPM2, protects brain against ischemia injury likely through inhibiting extrasynaptic NMDAR-induced large reduction in pro-survival signals.

TAT-EE3-treated mice exhibited reduced infarct volume and improved neurological deficit (ND) score (FIGS. 18C-18E). For the post-MCAO treatment, The protective effects of TAT-EE3 were evaluated at 24 h, 3 days, and 7 days. As shown in FIGS. 18C-18E, post-MCAO TAT-EE3 treatment reduced infarct volume and improved ND score in WT mice but did not produce further protective effects in the nM2KO mice, indicating that TAT-EE3 specifically targets TRPM2. For the long-term MCAO (7 days), ND score as well as behavioral changes were evaluated using rotarod test at day 1, day 3, and day 7. TAT-EE3 improved ND scores at day 1, day 3, and day 7 (FIG. 18F) and inhibited the reduction of “latency to fall” time (FIG. 18G). These results suggest that TAT-EE3 disrupts TRPM2-NMDAR coupling (FIG. 8A), thereby protecting mice against ischemic stroke.

### Discussion

Herein, a previously unknown mechanism underlying ischemic brain stroke is shown: the up-regulated TRPM2 during ischemic stroke is physically and functionally coupled to NMDARs, which results in enhanced extrasynaptic NMDARs activity and thereby leads to increased excitotoxicity. It is revealed that the mechanism by which TRPM2 increases extrasynaptic NMDRRs excitotoxicity is through interaction between TRPM2 and PKCγ, which leads to an increased NMDARs protein trafficking and surface expression. Moreover, we identified the specific binding domain in the N-terminal of TRPM2 for coupling of TRPM2 with NMDARs and designed a membrane permeable disrupting peptide to uncouple TRPM2 from NMDARs. By disrupting the TRPM2-NMDAR interaction, the peptide TAT-EE3 not only inhibits OGD-induced excitotoxicity in vitro, but also efficiently protects mice against ischemic stroke injury in vivo. These results indicate that by exacerbating the excitotoxicity of NMDARs, TRPM2 converges the excitotoxic and non-excitotoxic Ca2+ signaling pathways in mediating neuronal death in ischemic stroke. This new mechanism may serve as a foundation for designing and developing effective strategies of future ischemic stroke therapies.

### TRPM2 in Neurons Plays a Key Role in Ischemic Injury by Exacerbating NMDARs Excitotoxicity

The Ca2+-permeable TRPM2 was discovered as an oxidative stress-activated non-selective cation channel (Hara, Y., Wakamori, M., Ishii, M., Macno, E., Nishida, M., Yoshida, T., Yamada, H., Shimizu, S., Mori, E., Kudoh, J., et al. (2002). LTRPC2 Ca2+-permeable channel activated by changes in redox status confers susceptibility to cell death. Molecular cell 9, 163-73; Perraud, A. L., Fleig, A., Dunn, C. A., Bagley, L. A., Launay, P., Schmitz, C., Stokes, A. J., Zhu, Q., Bessman, M. J., Penner, R., et al. (2001). ADP-ribose gating of the calcium-permeable LTRPC2 channel revealed by Nudix motif homology. Nature 411, 595-99; Sano, Y., Inamura, K., Miyake, A., Mochizuki, S., Yokoi, H., Matsushime, H., and Furuichi, K. (2001). Immunocyte Ca2+ influx system mediated by LTRPC2. Science 293, 1327-30), and has been recently considered as one of the few potential candidates of non-excitotoxic targets for ischemic stroke (Belrose, J. C., and Jackson, M. F. (2018). TRPM2: a candidate therapeutic target for treating neurological diseases. Acta pharmacologica Sinica 39, 722-32). Activation of TRPM2 requires a rise of intracellular Ca2+ and binding of ADPR to TRPM2 (Huang, Y., Winkler, P. A., Sun, W., Lu, W., and Du, J. (2018). Architecture of the TRPM2 channel and its activation mechanism by ADP-ribose and calcium. Nature 562, 145-49; Wang, L., Fu, T. M., Zhou, Y., Xia, S., Greka, A., and Wu, H. (2018). Structures and gating mechanism of human TRPM2. Science 362; Zhang, Z., Toth, B., Szollosi, A., Chen, J., and Csanady, L. (2018b). Structure of a TRPM2 channel in complex with Ca (2+) explains unique gating regulation. eLife 7). TRPM2 has been shown to play a role in ischemic stroke. However, the mechanisms by which TRPM2 mediates ischemic injury has been controversial. Some studies suggest that neuronal TRPM2 (Alim, I., Teves, L., Li, R., Mori, Y., and Tymianski, M. (2013). Modulation of NMDAR subunit expression by TRPM2 channels regulates neuronal vulnerability to ischemic cell death. The Journal of neuroscience: the official journal of the Society for Neuroscience 33, 17264-77; Jia et al., 2011), whereas others reported that TRPM2 expressed in immune cells (Gelderblom, M., Melzer, N., Schattling, B., Gob, E., Hicking, G., Arunachalam, P., Bittner, S., Ufer, F., Herrmann, A. M., Bernreuther, C., et al. (2014). Transient receptor potential melastatin subfamily member 2 cation channel regulates detrimental immune cell invasion in ischemic stroke. Stroke; a journal of cerebral circulation 45, 3395-02) contributes to ischemic injury. As all the previous studies used global TRPM2-KO (gM2KO), it is shown that neuron-specific TRPM2 knockout (nM2KO) and found that neuronal TRPM2 plays a key role in mediating ischemic brain injury. These results not only establish that TRPM2 expressed in neurons is critical in mediating ischemic injury, but also reveal a previously unknown mechanism that TRPM2 exacerbates NMDAR's excitotoxicity in mediating neuronal death during ischemic injury.

A Specific Domain Mediates Physical and Functional Coupling of TRPM2 with NMDARs and Exacerbation of NMDARs' Excitotoxicity

Cerebral ischemic injury is characterized by excitotoxicity caused by overactivation of NMDARs which leads to mitochondria dysfunction (Schinder, A. F., Olson, E. C., Spitzer, N.C., and Montal, M. (1996). Mitochondrial dysfunction is a primary event in glutamate neurotoxicity. The Journal of neuroscience: the official journal of the Society for Neuroscience 16, 6125-33), and Ca2+ overload resulted from both excitotoxic and non-excitotoxic Ca2+ signaling pathways (Choi, D. W. (2020). Excitotoxicity: Still Hammering the Ischemic Brain in 2020. Frontiers in neuroscience 14, 579953). The excitotoxic glutamate-dependent NMDAR channels have been the targets for stroke intervention for over 50 years (Choi, D. W. (2020). Excitotoxicity: Still Hammering the Ischemic Brain in 2020. Frontiers in neuroscience 14, 579953). TRPM2 is one of the glutamate-independent, non-excitotoxic Ca2+ channels which have emerged as potential targets for ischemic stroke in recent years (Tymianski, M. (2011). Emerging mechanisms of disrupted cellular signaling in brain ischemia. Nature neuroscience 14, 1369-73). The intriguing result in this study is that, as a non-excitotoxic Ca2+ permeable channel, TRPM2 exacerbates NMDARs' excitotoxicity, implying that intervention of TRPM2 may attenuates both non-excitotoxicity as well as excitotoxicity during ischemic stroke.

How could TRPM2 converge the non-excitotoxic Ca2+ signaling and NMDAR-dependent excitotoxic Ca2+ signaling pathways to mediate neuronal death during MCAO? It is shown herein that TRPM2 physically interacts with GluN2a and GluN2b in both heterologous expressing HEK293T cells and in mouse brains. The physical interaction leads to enhanced surface expression and increased current amplitude of NMDARs. More importantly, it is uncovered that the C-termini of GluN2a and GluN2b bind to a binding domain of TRPM2 at the N-terminus, the EE3 domain, a stretch of 16 residues containing three “EE” repeats separated by five residues. When the physical interaction is disrupted by mutations or truncation of the EE3, or by the disrupting peptide TAT-EE3, interaction of TRPM2 and NMDARs in HEK-293T cells and in neurons is abolished, leading to elimination of both the enhanced surface expression of NMDARs and the increased NMDARs channels activity (FIGS. 2A-2L-3A-3P).

TRPM2 Controls NMDAR Surface Expression Via Interacting with PKCγ During Ischemic Stroke

NMDARs have been shown to be regulated by PKC through modulating intrinsic channel properties and NMDARs trafficking (Carroll, R. C., and Zukin, R. S. (2002). NMDA-receptor trafficking and targeting: implications for synaptic transmission and plasticity. Trends in neurosciences 25, 571-77; Lan, J. Y., Skeberdis, V. A., Jover, T., Grooms, S. Y., Lin, Y., Araneda, R. C., Zheng, X., Bennett, M. V., and Zukin, R. S. (2001). Protein kinase C modulates NMDA receptor trafficking and gating. Nature neuroscience 4, 382-90; Xiong, Z. G., Raouf, R., Lu, W. Y., Wang, L. Y., Orser, B. A., Dudek, E. M., Browning, M. D., and MacDonald, J. F. (1998). Regulation of N-methyl-D-aspartate receptor function by constitutively active protein kinase C. Molecular pharmacology 54, 1055-63). Interestingly, it is found that the neuron specific isoform of PKC, PKCγ, interacts with TRPM2 through the N-terminal domain of TRPM2. More importantly, the interaction of TRPM2 and PKCγ was significantly increased in MCAO brains in vivo, and by H2O2 in the heterologous expressing HEK293T cells in vitro, indicating that oxidative stress and ischemic injury conditions promote TRPM2 and PKCγ association (FIGS. 4A-4R). Consistent with our results, oxidative stress induced interaction of PKCα with a non-functional shorter isoform of TRPM2 alternative slice variant, TRPM2-S, was reported in a previous study, albeit full length TRPM2 was found not to interact with PKCa (Hecquet, C. M., Zhang, M., Mittal, M., Vogel, S. M., Di, A., Gao, X., Bonini, M. G., and Malik, A. B. (2014). Cooperative interaction of trp melastatin channel transient receptor potential (TRPM2) with its splice variant TRPM2 short variant is essential for endothelial cell apoptosis. Circulation research 114, 469-79). As PKC modulates NMDARs trafficking, it is conceivable that increased interaction of PKCγ and TRPM2 under oxidative stress conditions underlies the mechanism of elevated surface expression of NMDARs in MCAO mouse brains.

PKC-mediated NMDAR trafficking to cell surface has been proposed with different mechanisms. Some studies demonstrated that PKC mediates NMDARs surface trafficking through phosphorylation of serine residues (Ser896 and Ser897) located at the close proximity to the ER-retention motifs of GluN1 (Horak, M., and Wenthold, R. J. (2009). Different roles of C-terminal cassettes in the trafficking of full-length NR1 subunits to the cell surface. The Journal of biological chemistry 284, 9683-91; Scott, D. B., Blanpied, T. A., Swanson, G. T., Zhang, C., and Ehlers, M. D. (2001). An NMDA receptor ER retention signal regulated by phosphorylation and alternative splicing. The Journal of neuroscience: the official journal of the Society for Neuroscience 21, 3063-72; Standley, S., Roche, K. W., McCallum, J., Sans, N., and Wenthold, R. J. (2000). PDZ domain suppression of an ER retention signal in NMDA receptor NR1 splice variants. Neuron 28, 887-98), whereas others showed that PKC-induced increase of NMDAR activity is not mediated by phosphorylation of NMDARs (Zheng, X., Zhang, L., Wang, A. P., Bennett, M. V., and Zukin, R. S. (1999). Protein kinase C potentiation of N-methyl-D-aspartate receptor activity is not mediated by phosphorylation of N-methyl-D-aspartate receptor subunits. Proceedings of the National Academy of Sciences of the United States of America 96, 15262-67). The latter is supported by another study showing that PKC-induced NMDAR trafficking is mediated by triggering auto-phosphorylation of CaMKII, which is associated with NMDARs (Yan, J. Z., Xu, Z., Ren, S. Q., Hu, B., Yao, W., Wang, S. H., Liu, S. Y., and Lu, W. (2011). Protein kinase C promotes N-methyl-D-aspartate (NMDA) receptor trafficking by indirectly triggering calcium/calmodulin-dependent protein kinase II (CaMKII) autophosphorylation. The Journal of biological chemistry 286, 25187-200). Nonetheless, it seems that phosphorylation function is important for PKC to mediate NMDAR trafficking regardless whether it directly phosphorylates NMDARs or indirectly phosphorylates associating partners of NMDARs. Indeed, whereas PKCγ co-expressed with TRPM2 and NMDARs caused enhancement of NMDARs surface expression, the dominate-negative PKCγ failed to do so (FIGS. 4A-4R). Moreover, PKC activator PMA induced higher NMDARs surface expression, whereas PKC inhibitor staurosporine blocked PKC induced effects (FIGS. 4A-4R). The effects of PKCγ in the absence of PMA could be attributed to the basal activity under cell culture conditions when cells are surrounded with various growth factors. As both PKCγ and GluN2a/GluN2b interact with the N-terminus of TRPM2 (FIGS. 3A-3P and 4A-4R), it is conceivable that under oxidative stress conditions, the increased binding of PKCγ to TRPM2 (FIGS. 5E and 5F) may bring PKCγ to a closer proximity of NMDARs or their interacting protein partners, therefore increases PKCγ induced phosphorylation and subsequently surface trafficking of NMDARs.

Another evidence supporting the notion of PKCγ-mediated TRPM2-NMDAR complex trafficking to cell surface is the inhibitory effects produced by endosidine2, an inhibitor of one component of the exocyst complex (Sans, N., Prybylowski, K., Petralia, R. S., Chang, K., Wang, Y. X., Racca, C., Vicini, S., and Wenthold, R. J. (2003). NMDA receptor trafficking through an interaction between PDZ proteins and the exocyst complex. Nature cell biology 5, 520-30), EXO70 (Zhang et al., 2016). NMDARs interact with exocyst (Sans, N., Prybylowski, K., Petralia, R. S., Chang, K., Wang, Y. X., Racca, C., Vicini, S., and Wenthold, R. J. (2003). NMDA receptor trafficking through an interaction between PDZ proteins and the exocyst complex. Nature cell biology 5, 520-30) for PKC-induced surface delivery via secretory pathway (Hirschberg, K., Miller, C. M., Ellenberg, J., Presley, J. F., Siggia, E. D., Phair, R. D., and Lippincott-Schwartz, J. (1998). Kinetic analysis of secretory protein traffic and characterization of golgi to plasma membrane transport intermediates in living cells. The Journal of cell biology 143, 1485-503). It was found herein that endosidine2 abolished TRPM2-induced enhancement of NMDARs surface expression, and inhibited NMDAR currents in the neurons from WT mice (FIGS. 1Q-1R and 13A-B), suggesting that TRPM2 induced NMDAR surface trafficking involves PKC activation-induced secretory pathway. Furthermore, disruption of the TRPM2-NMDAR interaction largely eliminates increases in surface expression of NMDARs as well as functional NMDAR currents elicited by PMA (FIG. 5A-5J), strongly supporting that the PKCγ/TRPM2/NMDARs complex is critical for NMDARs surface trafficking. Although further studies are needed to fully understand the exact mechanism by which PKCγ mediates trafficking of the TRPM2/NMDAR complex, we propose the following working model based on our results. Interactions of TRPM2 with NMDARs and PKCγ create a close proximity environment where releasing of NMDARs from ER and trafficking of NMDARs to cell surface are significantly increased under oxidative stress condition, conferring enhanced excitotoxicity during cerebral ischemic injury.

### Functional Coupling of TRPM2 to NMDARs Exacerbates Extrasynaptic Excitotoxicity During Ischemic Injury

These data show that functional coupling between TRPM2 and NMDARs appears to only exacerbate extrasynaptic NMDAR's excitotoxicity. Using the disrupting peptide TAT-EE3, it was found that TAT-EE3 not only effectively eliminated the increase of NMDAR surface expression and functional increase of NMDAR currents induced by PKC activation (FIGS. 5A-5J and 6A-6G), but also sufficiently eliminated OGD-induced excitotoxicity in vitro (FIG. 6A-6G), and significantly attenuated MCAO-induced neuron death in vivo (FIGS. 7A-7K). Intriguingly, TAT-EE3 prevented the decreases in PERK1/2 and pCREB levels in OGD-treated neurons and MCAO brains, a hallmark of pro-survival signaling pathway which can be shut off by extrasynaptic NMDAR activation during ischemic injury (Hardingham, G. E., and Bading, H. (2010). Synaptic versus extrasynaptic NMDA receptor signalling: implications for neurodegenerative disorders. Nature reviews Neuroscience 11, 682-96; Hardingham, G. E., Fukunaga, Y., and Bading, H. (2002). Extrasynaptic NMDARs oppose synaptic NMDARs by triggering CREB shut-off and cell death pathways. Nature neuroscience 5, 405-14). Similar to what we demonstrated, the pCREB level can be reduced by more than 50% 24 hrs after MCAO (Zhang, D., Jin, W., Liu, H., Liang, T., Peng, Y., Zhang, J., and Zhang, Y. (2020). ENT1 inhibition attenuates apoptosis by activation of cAMP/pCREB/Bcl2 pathway after MCAO in rats. Experimental neurology 331, 113362). The ability for TAT-EE3 and TRPM2 knockout to prevent the decreases in pCREB and pERK1/2 levels in MCAO mice strongly indicates that disruption of TRPM2 and NMDAR coupling largely eliminates extrasynaptic excitotoxicity during ischemic brain injury.

It is shown herein that the TRPM2-NMDAR coupling only exacerbates extrasynaptic NMDAR functions (FIGS. 7A-7K), as TRPM2 is absent from the synapse based on the synaptome databases (Bayes, A., Collins, M. O., Croning, M. D., van de Lagemaat, L. N., Choudhary, J. S., and Grant, S. G. (2012). Comparative study of human and mouse postsynaptic protcomes finds high compositional conservation and abundance differences for key synaptic proteins. PloS one 7, e46683). Other studies have also demonstrated a predominantly extrasynaptic distribution of TRPM2 in cultured hippocampal neurons (Olah, M. E., Jackson, M. F., Li, H., Perez, Y., Sun, H. S., Kiyonaka, S., Mori, Y., Tymianski, M., and MacDonald, J. F. (2009). Ca2+-dependent induction of TRPM2 currents in hippocampal neurons. The Journal of physiology 587, 965-979). Moreover, a previous study demonstrated that NMDAR expression in hippocampal slices from mice not subjected to MCAO was not influenced by TRPM2-KO (Xic, Y.-F., Belrose, J. C., Lei, G., Tymianski, M., Mori, Y., MacDonald, J. F., and Jackson, M. F. (2011). Dependence of NMDA/GSK-3B Mediated Metaplasticity on TRPM2 Channels at Hippocampal CA3-CA1 Synapses. Molecular Brain 4, 44), which is consistent with our results showing that NMDARs surface expression level in sham mice is indifferent between WT and TRPM2-KO (FIG. 1W). Thus, it is unlikely that TRPM2-KO can cause abnormal functions of NMDARs under normal physiological conditions. Indeed, TRPM2-KO mice were behaviorally indistinguishable from WT littermates (Yamamoto, S., Shimizu, S., Kiyonaka, S., Takahashi, N., Wajima, T., Hara, Y., Negoro, T., Hiroi, T., Kiuchi, Y., Okada, T., et al. (2008). TRPM2-mediated Ca2+ influx induces chemokine production in monocytes that aggravates inflammatory neutrophil infiltration. Nature medicine 14, 738-47), which is consistent with our observation that global TRPM2-KO as well as neuron-specific TRPM2-KO mice behave as same as their WT littermates. Furthermore, TRPM2 only enhanced NMDAR function under ischemic injury conditions, which promote PKCγ to interact with TRPM2 and subsequently NMDARs trafficking to cell surface. Therefore, disrupting the interaction between TRPM2 and NMDARs to specifically target extrasynaptic NMDAs and to mitigate ischemic stroke will unlikely generate side effects as caused by directly antagonizing NMDARs.

### Disrupting Peptide TAT-EE3 Protects Neurons Against Ischemic Injury In Vitro and In Vivo

A most exciting result in this study is the effectiveness of the disruption of NMDARs-TRPM2 coupling in protecting mice against ischemic brain stroke. Membrane permeable peptides such as TAT-fused peptides have been well characterized and are considered as powerful tools for both medical applications and fundamental basic research (Aarts et al., 2002; Kilic et al., 2006; Schulien et al., 2020; Shimizu et al., 2016; Xic et al., 2020). By disrupting the TRPM2-NMDAR interaction, TAT-EE3 effectively eliminated the enhanced extrasynaptic toxicity of NMDARs induced by TRPM2. In neurons treated with 10 μM TAT-EE3 and in mice administrated with 100 nmol/Kg TAT-EE3, it was found that TAT-EE3 significantly reduced excitotoxicity induced by OGD, prevented the reduction of pCREB and pERK1/2 level in OGD-treated neurons and MCAO mice, and reduced infarct volume as well as neurological deficits in MCAO mice (FIGS. 7A-7K and 14A-14C). These results indicate that disrupting TRPM2 and NMDAR coupling is a promising therapeutic strategy for ischemic stroke.

NMDARs interact various different proteins, including receptors and ion channels in addition to the interacting proteins for NMDAR trafficking and stabilization (Petit-Pedrol, M., and Groc, L. (2021). Regulation of membrane NMDA receptors by dynamics and protein interactions. The Journal of cell biology 220). In comparison with those of other NMDAR interacting ion channels, including ASIC1a (Gao et al., 2005), pannexin (Bargiotas, P., Krenz, A., Hormuzdi, S. G., Ridder, D. A., Herb, A., Barakat, W., Penucla, S., von Engelhardt, J., Monyer, H., and Schwaninger, M. (2011). Pannexins in ischemia-induced neurodegeneration. Proceedings of the National Academy of Sciences of the United States of America 108, 20772-77; Weilinger, N. L., Lohman, A. W., Rakai, B. D., Ma, E. M., Bialecki, J., Maslicieva, V., Rilea, T., Bandet, M. V., Ikuta, N. T., Scott, L., et al. (2016). Metabotropic NMDA receptor signaling couples Src family kinases to pannexin-1 during excitotoxicity. Nature neuroscience 19, 432-42), BKCa (Zhang, J., Guan, X., Li, Q., Meredith, A. L., Pan, H. L., and Yan, J. (2018a). Glutamate-activated BK channel complexes formed with NMDA receptors. Proceedings of the National Academy of Sciences of the United States of America 115, E9006-14) and TRPM4 (Yan, J., Bengtson, C. P., Buchthal, B., Hagenston, A. M., and Bading, H. (2020). Coupling of NMDA receptors and TRPM4 guides discovery of unconventional neuroprotectants. Science 370), there are several unique aspects about the TRPM2-NMDAR interaction. First, different from other interacting ion channels, TRPM2 is an oxidative stress-activated Ca2+-permeable channel; second, as an ion channel interacting with NMDARs, TRPM2 alters NMDAR surface expression by enhancing NMDAR surface trafficking; third, TRPM2 orchestrates PLCγ to NMDAR interacting complex to enhance NMDAR surface trafficking; fourth, TRPM2 exacerbates extrasynaptic NMDAR function to increase neuronal death. Since TRPM2 is a non-excitotoxic Ca2+ permeable channel, the TRPM2-NMDAR interaction-induced exacerbated NMDARs' excitotoxicity makes TRPM2 a molecule that converges non-excitotoxic and excitotoxic Ca2+ signaling pathways, which may serve as an effective target for ischemic stroke.

### Translational Implications

The data herein provide a new strategy of targeting excitotoxicity without directly antagonizing NMDARs. Excitotoxicity by glutamate acting on NMDARs has long been established as the dominant conceptual model underlying neuronal cell death associated with ischemic stroke. However, the outcome from clinical trials of directly antagonizing NMDARs by NMDAR antagonists has been disappointing (Choi, D. W. (2020). Excitotoxicity: Still Hammering the Ischemic Brain in 2020. Frontiers in neuroscience 14, 579953). Adverse effect of inhibiting NMDARs' physiological functions is one of many factors contributing to the failed NMDARs antagonist in translational applications. Herein it is shown that disrupting the physical and functional coupling of NMDARs and TRPM2 can largely eliminate the extrasynaptic toxicity of NMDARs during ischemic injury while preserving the pro-survival synaptic NMDAR function. This may represent an effective strategy for future designing and developing of treatments for ischemic stroke.

The results herein also provide novel insights about the mechanisms by which non-excitotoxic Ca2+ permeable channels contribute to ischemic stroke. The unsuccessful clinical trials of NMDAR antagonists for ischemic stroke have promoted research focusing on the non-excitotoxic Ca2+ permeable channels such as TRP channels, ASICs channels, and P2X4 in recent years (Tymianski, M. (2011). Emerging mechanisms of disrupted cellular signaling in brain ischemia. Nature neuroscience 14, 1369-73). These results indicate that, in addition to Ca2+ permeating function, TRPM2 mediates excitotoxic effects by interacting with NMDARs during ischemic stroke. Thus, targeting TRPM2 provides a convergent inhibitory strategy to eliminate both non-excitotoxic and excitotoxic Ca2+ signal-mediated neuronal toxicity in ischemic stroke.

These results highlight an overlooked factor that might have contributed to the gap between the effectiveness in preclinical research and non-effective outcome in clinical trials. Gene knockout is commonly used in determining the role of a specific gene in ischemic stroke animal models. Whereas deletion of TRPM2 is an effective way to abolish the deleterious effects of TRPM2 in MCAO mice, the protective effects of TRPM2-KO in MCAO mice are attributed to both abolishing TRPM2 channel ion-conducting function and eliminating the TRPM2-NMDAR interaction-associated deleterious effects. These protective effects will unlikely be recapitulated in patients with ischemic brain stroke if TRPM2 channel blockers were used for treatment, as the TRPM2-NMDAR coupling mediated deleterious effects will unlikely be influenced by TRPM2 channel inhibitors. Such “hidden” effects similar to the TRPM2-NMDAR interaction-mediated ones may also exist in other channels (Yan, J., Bengtson, C. P., Buchthal, B., Hagenston, A. M., and Bading, H. (2020). Coupling of NMDA receptors and TRPM4 guides discovery of unconventional neuroprotectants. Science 370). Thus, identification of the “hidden” effects, the functional coupling of non-excitotoxic Ca2+ permeable channels with NMDARs and the associated excitotoxicity may help uncover better targets and therefore promise better outcome from clinical trials in the future.

In summary, the inventors of the present invention discovered that TRPM2 in neurons plays a key role in mediating the deleterious effects of TRPM2 in ischemic brain stroke. The inventors uncovered that physical and functional coupling of TRPM2 with NMDARs leads to enhanced extrasynaptic NMDAR toxicity under oxidative stress condition. Interaction of TRPM2 with PKCγ underlies the mechanism by which TRPM2 mediates enhancement of NMDAR surface expression and functional increase of NMDAR currents. In addition, the inventors identified a specific NMDAR-binding domain of TRPM2, and designed a membrane permeable disrupting peptide TAT-EE3. It was demonstrated that uncoupling TRPM2 from NMDARs leads to protective effects in vitro and in vivo. As TRPM2 is a non-excitotoxic Ca2+ permeable channel, these results provide a new strategy that targeting TRPM2 can eliminate ischemic neuronal toxicity mediated by both non-excitotoxic and excitotoxic Ca2+ signaling pathways and protect animals against ischemic stroke.

### Methods and Materials:

TABLE 1
KEY RESOURCES TABLE

| | | |
|---|---|---|
| REAGENT or RESOURCE | SOURCE | IDENTIFIER |

Antibodies

| | | |
|---|---|---|
| Rabbit polyclonal antibodies to TRPM2 | Novus | NB110-81601 |
| Rabbit polyclonal antibodies to TRPM2 N | Abmart | 634-1-1-R1 |
| terminal part | | |
| Rabbit polyclonal antibodies to TRPM2 C | Abmart | 634-2-1-R1 |
| terminal part | | |
| Rabbit polyclonal antibodies to GluN1 | Cell Signaling | 5704S |
| | Technology | |
| Rabbit polyclonal antibodies to GluN2A | Cell Signaling | 4205S |
| | Technology | |
| Rabbit polyclonal antibodies to GluN2B | Cell Signaling | 4207S |
| | Technology | |
| Mouse monoclonal antibodies to flag | Sigma- | F3165 |
| | Aldrich | |
| Rabbit polyclonal antibodies to phosphor- | Cell Signaling | 9198S |
| CREB(Ser133) | Technology | |
| Rabbit polyclonal antibodies to p44/42 | Cell Signaling | 9102S |
| MAPK (ERK1/2) | Technology | |
| Rabbit polyclonal antibodies to phospho- | Cell Signaling | 4377T |
| p44/42 MAPK | Technology | |
| (ERK1/2) (Tyr202/204) | | |
| Rabbit polyclonal antibodies to PKC-γ | Cell Signaling | 59090S |
| | Technology | |
| Rabbit polyclonal antibodies to Pan-cadherin | Cell Signaling | 4068S |
| | Technology | |
| Rabbit polyclonal antibodies to GAPDH | Cell Signaling | 2118S |
| | Technology | |
| Rabbit polyclonal antibodies to β-tubulin | Cell Signaling | 4820S |
| | Technology | |
| Rabbit polyclonal antibodies to NeuN | Abcam | ab187477 |
| Goat anti-rabbit IgG-FITC | Santa Cruz | sc-2012 |
| | Biotechnology | |
| Goat anti-mouse IgG-rhodamine | Thermal | 31160 |
| | Fisher | |
| | Scientific | |

Chemicals and peptides

| | | |
|---|---|---|
| 2,3,5-Triphenyltetrazolium chloride | Sigma- | T-8877 |
| | Aldrich | |
| N-Methyl-D-aspartic acid | Tocris | 0114 |
| Glutamate | Sigma- | 49621 |
| | Aldrich | |
| Glycine | Sigma- | 50046 |
| | Aldrich | |
| Bicuculine | TCI | B1890 |
| 4-Aminopyridine | Sigma- | A-0152 |
| | Aldrich | |
| MK-801 | Sigma- | M107 |
| | Aldrich | |
| Phorbol-12-myristate-13-acetate | Sigma- | 524400 |
| | Aldrich | |
| 30% Hydrogen Peroxide | Thermal | 200745 |
| | Fisher | |
| | Scientific | |
| NP40 | Thermal | 28324 |
| | Fisher | |
| | Scientific | |
| Triton ™ X-100 | Sigma- | T-9284 |
| | Aldrich | |
| Bovine Serum Albumin | Sigma- | 9048-46-8 |
| | Aldrich | |
| Goat Serum | Thermal | 16210-064 |
| | Fisher | |
| | Scientific | |
| Rhodamine-123 | Thermal | R302 |
| | Fisher | |
| | Scientific | |
| Fura-2 AM | Thermal | F1221 |
| | Fisher | |
| | Scientific | |
| Ionomycin | Sigma- | I0634 |
| | Aldrich | |
| Pluronic ™ F-127 | Thermal | P3000MP |
| | Fisher | |
| | Scientific | |
| Proteinase inhibitors | Sigma- | 539131-10VL |
| | Aldrich | |
| Phosphatase inhibitors | Thermal | 78428 |
| | Fisher | |
| | Scientific | |
| Laemmli Sample Buffer | BIO-RAD | 1610737 |
| Protein A/G PLUS-Agarose | Santa Cruz | sc-2003 |
| | Biotechnology | |
| TAT-SC | Genescript | N/A |
| TAT-EE₃ | Genescript | N/A |

Chemicals and peptides
All chemicals for making artificial cerebrospinal fluid (aCSF; see below)
and recording solution (see below) were purchased from Sigma-Aldrich.
Plasmids and enzymes

| GluN1a | Addgene | 17928 |
|---|---|---|
| GluN2A | Addgene | 17924 |
| GluN2B | Addgene | 17925 |
| PKC-γ | Addgene | 112266 |
| PKC-γ-DN | Addgene | 21239 |
| pcDNA4/TO-FLAG-hTRPM2 | Dr. Sharenberg AM (University | |
| | of Washington, Seattle) | |
| | (Perraud et al., 2003) | |
| Xbal | BioLabs | R0145S |
| Xhol | BioLabs | R0146S |
| BamHI | BioLabs | R3136S |
| DpnI | BioLabs | R0176S |
| EcoRI | BioLabs | R3101S |
| T4 DNA ligase | Thermo Fisher | 2148085 |
| | Scientific | |
| PfuUltra HF | Agilent | 600380-51 |
| Q5 ® High Fidelity DNA Polymerase | BioLabs | M0491S |

Cell Culture

| | | |
|---|---|---|
| Dulbecco's Modified Eagle's medium | Thermo Fisher | 12100-038 |
| | Scientific | |
| Bovine Calf Serum | HyClone | SH30541.03 |
| Penicillin/streptomycin | Thermo Fisher | 15140-122 |
| | Scientific | |
| 2.5% trypsin | Thermo Fisher | 15090-046 |
| | Scientific | |
| Neurobasal ® medium | Thermo Fisher | 21103-049 |
| | Scientific | |
| B27 Medium | Thermo Fisher | 17504-044 |
| | Scientific | |
| Horse serum | Thermo Fisher | 16050114 |
| | Scientific | |
| l-glutamine | Thermo Fisher | 25030-081 |
| | Scientific | |
| Cytosine arabinoside | Sigma-Aldrich | C1768 |
| Poly-l-lysine | | P4707 |

Critical Commercial Assays

| | | |
|---|---|---|
| Lipofectamine ® 3000 Transfection Kit | Thermo Fisher | 2232162 |
| | Scientific | |
| Pierce ™ Rapid Gold BCA Protein Assay Kit | Thermo Fisher | A53225 |
| | Scientific | |
| Pierce ® Cell Surface Native Membrane | Thermo Fisher | 89881 |
| Protein Extraction Kit | Scientific | |
| ProteoExtract ™ Native Membrane Protein | Calbiochem | 444810 |
| Extraction Kit | | |
| Fisher Healthcare Tissue-Plus ™O.C.T. | Thermo Fisher | 23-730-571 |
| Compound | Scientific | |
| ProLong ™ Gold Antifade Mountant | Thermo Fisher | P10144 |
| | Scientific | |
| QIAprep ®Spin Miniprep Kit | QIAGEN | 27106 |
| QIAGEN Plasmid Maxi Kit | QIAGEN | 12163 |
| Qiaquick PCR Purification Kit | QIAGEN | 28104 |
| In Situ Cell Death Detection Kit | Millipore | 11684795910 |
| | Sigma | |
| Oligonucleotides | M | |
| Primers for TRPM2 subcloning and | This study | N/A |
| mutagenesis, see Table 2 | | |
| Primers for GluN2a and GluN2b subcloning | This study | N/A |
| and mutagenesis, see Table 2 | | |

Software

| | | |
|---|---|---|
| GraphPad Prism 6.0 | GraphPad | N/A |
| | Software | |
| Biorender | Biorender | N/A |
| Adobe Illustrator | Adobe | N/A |
| NIS Elements AR4 | Nikon | N/A |
| pClamp 9.2 | Molecular | N/A |
| | Devices | |

### Animal Models Animal Care

All the experimental mice bred and hosted in the animal facility building of University of Connecticut School of Medicine (UCONN Health) were fed with standard chow diet and water ad libitum. Standard housing conditions were maintained at a controlled temperature with a 12-h light/dark cycle. All experimental procedures and protocols were approved by the Institutional Animal Care and Use Committee (IACUC) of University of Connecticut School of Medicine (animal protocol: AP-200135-0723), and were conducted in accordance with the U.S. National Institutes of Health Guidelines for the Care and Use of Laboratory Animals.

### Knockout of TRPM2 (TRPM2-KO)

The global TRPM2 knockout (TRPM2-KO, or gM2KO) mice were generated by Dr. Yasuo Mori's lab at Kyoto University Japan. The deletion of Trpm2 was developed in C57B6J mouse by replacing the third exon (S5-S6 linker in the pore domain) with a neomycin coding region. The knockout mice exhibited no differences in behavior or impairment in breeding, compared to wild type (WT) C57BJ6 mice (Yamamoto, S., Shimizu, S., Kiyonaka, S., Takahashi, N., Wajima, T., Hara, Y., Negoro, T., Hiroi, T., Kiuchi, Y., Okada, T., et al. (2008). TRPM2-mediated Ca2+ influx induces chemokine production in monocytes that aggravates inflammatory neutrophil infiltration. Nature medicine 14, 738-47). TRPM2-KO mice were back-crossed to C57BL/6 mice for ≥10 generations before being used for experiments.

The neuron specific knockout of TRPM2 (nM2KO) was generated by breeding TRPM2fl/fl mice with Nestin-Cre ((B6.Cg-Tg) Nes-cre)1kln/J: 003771; JAX laboratory). TRPM2fl/fl mice were generated by Dr. Barbara Miller (Miller, B. A., Wang, J., Hirschler-Laszkiewicz, I., Gao, E., Song, J., Zhang, X. Q., Koch, W. J., Madesh, M., Mallilankaraman, K., Gu, T., et al. (2013). The second member of transient receptor potential-melastatin channel family protects hearts from ischemia-reperfusion injury. American journal of physiology Heart and circulatory physiology 304, H1010-22) (Penn State University, Pennsylvania). The exons 21 and 22 encoding transmembrane domain 5 and 6 and pore loop were flanked by loxp recombination sites and will be deleted by Cre recombinase (Miller, B. A., Wang, J., Hirschler-Laszkiewicz, I., Gao, E., Song, J., Zhang, X. Q., Koch, W. J., Madesh, M., Mallilankaraman, K., Gu, T., et al. (2013). The second member of transient receptor potential-melastatin channel family protects hearts from ischemia-reperfusion injury. American journal of physiology Heart and circulatory physiology 304, H1010-22). The mice were back-crossed with C57BL/6 mice for ≥10 generations before being used for experiments. The TRPM2flox/flox (TRPM2fl/fl) with Cre+ mice and TRPM2fl/fl with Cre-mice from the same litters were paired for experiments throughout the manuscript.

The inducible global knockout was also generated by using TRPM2fl/fl mice breeding with global Cre, Rosa26-CreERT2 (B6.129-Gt (ROSA) 26Sortm1(crc/ERT2)Tyj/J: 008463; JAX laboratory). Knockout was induced by Tamoxifen treatment and confirmed by genotyping. The mice were backcrossed with C57BL/6 mice for ≥10 generations before being used for experiments.

### Middle Cerebral Artery Occlusion (MCAO)

Eight-to nine-week-old male mice (˜25 g) were subjected to right middle cerebral artery occlusion (MCAO) for 120 min followed by 24 hours of reperfusion. The genotype information was blinded to the surgeon who conduct the surgeries. MCAO surgery was performed as previously described (Liu, F., and Mccullough, L. D. (2014). The middle cerebral artery occlusion model of transient focal cerebral ischemia. Methods in molecular biology 1135, 81-93; Wu, L. J., Wu, G., Akhavan Sharif, M. R., Baker, A., Jia, Y., Fahey, F. H., Luo, H. R., Feener, E. P., and Clapham, D. E. (2012). The voltage-gated proton channel Hvl enhances brain damage from ischemic stroke. Nature neuroscience 15, 565-73). In brief, mice were anesthetized with 2% isoflurane (vol/vol) in 100% oxygen and the anesthesia was maintained with 1.5% isoflurane during surgery through nose cone (Harvard Apparatus). The unilateral right middle cerebral artery (MCA) occlusion was carried out by advancing a silicone-coated 6-0 monofilament (Doccol Corporation, Sharon, MA) 10 to 11 mm from internal carotid artery bifurcation via an external carotid artery incision (Chiang, T., Messing, R. O., and Chou, W. H. (2011). Mouse model of middle cerebral artery occlusion. Journal of visualized experiments: JoVE). Mouse body temperature was monitored by a rectal temperature probe and maintained at ˜ 37° C. with an automatic temperature-regulating heating pad connected to animal temperature controller (TCA T-2DF, Physitemp). Cerebral blood flow was monitored after occlusion as well as after reperfusion. The bregma was exposed and the skull bone countersunk at two 3×3-mm areas over both MCA supply territories for bilateral monitoring of local cortical blood flow. Successful occlusion was confirmed by 85% reduction of cerebral blood flow monitored by Laser Doppler Flowmetry (LDF) with laser Doppler blood FlowMeter (Moor-VMS-LDF1, Moor Instrument, Dever, UK). Sham control mice underwent the same procedure but without insertion of filament to occlude the MCA.

### Neurological Deficit Score Evaluation

After 24 hours of poststroke, neurological deficit was scored based on previously reported criteria (Longa, E. Z., Weinstein, P. R., Carlson, S., and Cummins, R. (1989). Reversible middle cerebral artery occlusion without craniectomy in rats. Stroke; a journal of cerebral circulation 20, 84-91). In brief, score 0 represents no neurological deficit; score 1 represents failure to extend left paw; Score 2 represents circling to the left; score 3 represents falling to the left; score 4 represents inability of spontaneously walking and decreased level of consciousness; and score 5 represents death due to brain ischemia. The observer to score the neurological deficit was an experienced observer and blinded by the group assignment and genotype information. If the animal score was 0 or 5, it was removed from the study.

### Infarct Volume Assessment by Triphenyl Tetrazolium Chloride (TTC) Staining

Tetrazolium chloride (Sigma-Aldrich, T-8877) was dissolved in PBS at a concentration of 20 mg/ml 30 min prior to use. Post-stroke mice were euthanized and brains were frozen at −80° C. for 5 min, cut into coronary slices at a thickness of 1 mm. Brain slices were stained with 2% TTC (vol/vol) for 20 min, and then washed using PBS for 3 times, and fixed in 10% Neutral buffered formalin for later scanning. TTC labels non-injured tissue, leaving the infarct area white. The stained slices were scanned for data analysis using ImageJ software. The infarct volume was calculated and presented as a percentage of total brain volume (Ren, M., Senatorov, V. V., Chen, R. W., and Chuang, D. M. (2003). Postinsult treatment with lithium reduces brain damage and facilitates neurological recovery in a rat ischemia/reperfusion model. Proceedings of the National Academy of Sciences of the United States of America 100, 6210-15).

### Antibodies, Chemicals and Reagents

Rabbit polyclonal antibodies to TRPM2 (1:500 in 5% BSA for WB, 1:50 in protein extraction for IP); Rabbit polyclonal antibodies to GluN1 (1:1000 in 5% BSA for WB, 1:50 in protein extraction for IP); Rabbit polyclonal antibodies to GluN2A (1:1000 in 5% BSA for WB, 1:50 in protein extraction for IP). Rabbit polyclonal antibodies to GluN2B (1:1000 in 5% BSA for WB, 1:50 in protein extraction for IP); Mouse polyclonal antibodies to flag (1:5000 in 5% BSA for WB): Rabbit polyclonal antibodies to GFP (1:2000 in 5% BSA for WB); Rabbit polyclonal antibodies to CREB (1:2000 in 5% BSA for WB); Rabbit polyclonal antibodies to phosphor-CREB (Ser133) (1:2000 in 5% BSA for WB); Rabbit polyclonal antibodies to p44/42 MAPK (ERK1/2) (1:2000 in 5% BSA for WB); Rabbit polyclonal antibodies to phospho-p44/42 MAPK (ERK1/2) (Tyr202/204) (1:2000 in 5% BSA for WB); Rabbit polyclonal antibodies to PKC-γ (1:5000 in 5% BSA for WB); Rabbit polyclonal antibodies to Pan-cadherin (1:5000 in 5% BSA for WB); Rabbit polyclonal antibodies to GAPDH (1:5000 in 5% BSA for WB); Rabbit polyclonal antibodies to β-tubulin (1:5000 in 5% BSA for WB; Rabbit polyclonal antibodies to NeuN (1:50 in 5% BSA and 15% goat serum for immunofluorescence staining); Prolong® Gold antifade reagent with DAPI (Life technologies, P36935), HRP-linked anti-rabbit IgG (1:10000 in 5% BSA for WB); HRP-linked anti-mouse-IgG (1:10000 in 5% BSA for WB). Tetrazolium chloride (Sigma-Aldrich, T-8877); NMDA (Tocris, 0114); Glutamate (Sigma-Aldrich, 49621); Glycine (Sigma-Aldrich, 50046); Bicuculine (TCI, B1890); 4-AP (Sigma-Aldrich, A-0152); MK-801 (Sigma-Aldrich, M107); PMA (Sigma-Aldrich, 524400); 4-α-PMA (Sigma-Aldrich, P128); H2O2 (Thermal Fisher Scientific, 200745); NP40 (Thermal Fisher Scientific, 28324), Triton™ X-100 (T-9284), Bovine Serum Albumin (Sigma-Aldrich, 9048-46-8), Goat Serum (Thermal Fisher Scientific, 16210-064). All chemicals for making artificial cerebrospinal fluid (aCSF; see below) and recording solution (see below) were purchased from Sigma-Aldrich.

### Membrane Permeable Peptide TAT-EE3 for Disrupting TRPM2 and NMDARs Coupling and Scramble Control TAT-SC Peptides

TAT-SC (sequence: YGRKKRRQRRR VILLKDHTLEYPVF
(SEQ ID NO: 11))
and
TAT-EE3 (sequence: YGRKKRRQRRREEDTDSSEEMLALAEE
(SEQ ID NO: 4))

were ordered from GenScript Biotech, and dissolved in PBS to make a stock concentration at 10 mM. HEK-293T cells or isolated neurons were treated with TAT-SC or TAT-EE3 at a concentration of 10 μM for at least 8 h prior to use. Mice were injected with TAT-SC or TAT-EE3 intraperitoneally (ip) at a dose of 100 nmol/kg.

### Plasmids and Enzymes

GluN1a (Addgene, 17928), GluN2A (Addgene, 17924), GluN2B (Addgene, 17925), PKC-γ (Addgene, 112266), PKC-γ-DN (Addgene, 21239). The pcDNA4/TO-FLAG-hTRPM2 construct was a kind gift from Dr. Sharenberg A M (University of Washington, Seattle (Perraud, A. L., Schmitz, C., and Scharenberg, A. M. (2003). TRPM2 Ca2+ permeable cation channels: from gene to biological function. Cell calcium 33, 519-31).

XbaI (BioLabs, R0145S), BamHI (BioLabs, R3136S), XhoI (BioLabs, R0146S), DpnI (BioLabs, R0176S), EcoRI (BioLabs, R3101S) and T4 DNA ligase (Thermal Fisher Scientific, 2148085), PfuUltra HF (Agilent, 600380-51), and Q5® High-Fidelity DNA Polymerase (Biolabs, M0491S) were used to generating different deletion or mutation constructs.

### Subcloning

For TRPM2, subcloning of N terminus (1-727) was achieved by introducing a stop codon (A2282T) by PCR using PfuUltra HF. C terminal of TRPM2 was amplified by PCR using Q5® High-Fidelity DNA Polymerase, cut by EcoRI and XbaI, and inserted into EGFP-C3 vector. To look for the binding part in N terminus of TRPM2, a series of stop codons were introduced by PCR using PfuUltra HF (C1831A, C1994T, G2138T, A2090T). EE3 motif was deleted by PCR using Q5® High-Fidelity DNA Polymerase. EE was mutated to QQ by PCR using PfuUltra HF (G2093C, G2096C; G2117C, G2120C; G2138C, G2141C). For GluN2A and GluN2B, C terminal was amplified by PCR using Q5® High-Fidelity DNA Polymerase, cut by EcoRI and XbaI, and inserted into EGFP-C3 vector. Deletion of C terminus of GluN2A and GluN2B was achieved by introducing a stop codon by PCR using PfuUltra HF (G4371T for GluN2B and G4518T for GluN2B). Deletion of the KKR motif in GluN2a and GluN2b were achieved by PCR using Q5 Site-Directed Mutagenesis Kit based on the instructions.

For E. coli expression system, EE3 containing segment and EQE containing segment in TRPM2 were amplified by PCR using Q5High-Fidelity DNA Polymerase, cut by KpnI and NotI, and inserted into a homemade His6-tagged vector (SEQ ID NO: 10); and KKR containing segment in GluN2a and GluN2b were amplified by PCR using Q5 High-Fidelity DNA Polymerase, cut by KpnI and NotI, and inserted into a homemade GST-tagged vector.

The information of all the primers is listed in the Table 2.

E. coli Expression and Purification of Proteins

The human GluN2a and GluN2b genes were cloned into a modified pGEX vector containing a removable tobacco etch virus (TEV) protease recognition site. The GluN2a (residues 1208-1317; MW: 16.7 kDa) and GluN2b (residues 1212-1322; MW: 16.7 kDa) proteins were expressed in Escherichia coli BL21 (DE3) cells grown to an OD600 (optical density at 600 nm) between 0.8-1.0 at 37° C. followed by induction of protein expression at 21° C. overnight using 0.5 mM isopropyl-D-1-thiogalactopyranoside (IPTG). Cells were harvested by centrifugation, resuspended by lysis buffer containing 25 mM Tris-HCl (pH 8.0), 200 mM NaCl, 1% phenylmethylsulfonyl fuoride (PMSF) and 2 mM dithiothreitol (DTT), and lysed by using high-pressure homogenization (Avestin EmulsiFlex C3). The lysates were clarifed by centrifugation at 30,000 rpm at 4° C. for 30 min, and the supernants were applied to a Glutathione Sepharose 4B column (GE Healthcare). After being extensively washed with lysis buffer, the GST-tagged GluN2a and GluN2b fusion proteins were eluted with a buffer containing 25 mM Tris-HCl (pH 8.0), 200 mM NaCl, 15 mM reduced glutathione and 2 mM DTT. The proteins were concentrated using an Amicon stirred ultrafitration cell unit with a 10-kDa cutoff membrane (EMD Millipore) and stored at −80° C. until use.

The human wild-type TRPM2 (residues 644-760; ˜ 16.7 kDa) and its EQE mutant were cloned into a modifie pET15b vector containing a removable TEV protease recognition site. The proteins were expressed in E. coli BL21 (DE3) as described above. Cell pellets were resuspended in denaturing buffer containing 25 mM Tris-HCl (pH 8.0), 300 mM NaCl, 1% PMSF and 6 M Urea and lysed by high-pressure homogenization. The lysates were clarified by centrifugation at 30,000 rpm at 4° C. for 30 min, and the supernants were applied to a Ni2+-nitrilotriacetic acid (NTA) column (GE Healthcare). After being extensively washed with 25 mM imidazole in the denaturing buffer, the His6-tagged (SEQ ID NO: 10) TRPM2 fusion proteins were eluted with a buffer containing 25 mM Tris-HCl (pH 8.0), 300 mM NaCl and 250 mM imidazole. To refold the TRPM2 proteins, the denatured samples were dialyzed at 4° C. overnight against two changes of a buffer without urea [25 mM Tris-HCl (pH 8.0), 200 mM NaCl and 2 mM DTT]. The proteins were concentrated using an Amicon stirred ultrafitration cell unit with a 10-kDa cutoff membrane (EMD Millipore) and stored at −80° C. until use.

### In Vitro Protein-Protein Direct Binding Assay

About 1-10 μg of purified proteins were added into 1 mL freshly prepared binding buffer (25 mM HEPES, 100 mM NaCl, 0.01% Triton X-100 and 5% glycerol) for in vitro direct binding at 4° C. for overnight. Then Co-immunoprecipitation assay was performed using either anti-GST or anti-His antibodies at 1:50 dilution as detailed described in the Co-immunoprecipitation section. The precipitated proteins were detected by coommassie blue staining and western blot.

### Cell Culture and Transfection

HEK293T cells were cultured in Dulbecco's Modified Eagle's medium (DMEM) (Thermal Fisher Scientific, 12100-038) supplemented with 10% BGS (HyClone, SH30541.03) and 0.5% penicillin/streptomycin (Thermal Fisher Scientific, 15140-122) at 37° C. and 5% CO2. 8 h prior to transfection, culture medium was replaced with DMEM supplemented only with 2.5% BGS. Cells were transfected when at a confluence about 80-90% using Lipofectamine® 3000 Transfection Kit (Thermal Fisher Scientific, 2232162) based on instruction.

### Neuron Isolation and Culture

Mice pups at P0 were euthanized based on animal protocol. Whole brain was dissected out immediately and immersed in ice-cold Hank's Balanced Salt Solution (HBSS). Meninges were removed thoroughly, and tissue of different brain areas was taken based on purposes. Brain tissue was cut into small pieces and digested with 0.25% trypsin (Thermal Fisher Scientific, 15090-046) in HBSS at 37° C. for 20 min. Digestion solution was quickly removed, and tissue pellets are washed with Neurobasal® Medium (Thermal Fisher Scientific, 21103-049) for 3 times. Cells were resuspended with appropriate amount of Neurobasal® Medium supplemented with 2% B27® supplement (Thermal Fisher Scientific, 17504-044), 3% horse serum (Thermal Fisher Scientific, 16050114), 0.25% L-glutamine (Thermal Fisher Scientific, 25030-081) and 1% penicillin/streptomycin (Thermal Fisher Scientific, 15140-122). Isolated cells were counted and plated on coverslips pre-coated with poly-L-lysine (Sigma-Aldrich, P4707) at a density about 500×103 cells/cm2 for OGD and H2O2 treatment, and 100×103 cells/cm2 for current recording. Cytosine arabinoside (Sigma-Aldrich, C1768) was added to maintain a concentration at 1 μM to inhibit the proliferation of non-neuronal cells. 24 h after plating, culture medium was changed to Neurobasal® Medium supplemented with 2% B27® supplement, 0.25% L-glutamine and 1% penicillin/streptomycin. The concentration of Cytosine arabinoside (araC) was increased to 2 μM. Medium was changed every 3 days. OGD and H2O2 treatment was conducted at 7th day of culture, and current recording was conducted at 7th, 10th and 14th day of culture.

### Oxygen-Glucose Deprivation

Oxygen-glucose deprivation (OGD) was achieved by replace the glucose in aCSF with sucrose, and 95% N2 and 5% CO2 was used to equilibrate sucrose-aCSF to displace oxygen. This condition typically yielded a pO2 of <5 mm Hg in the imaging chamber (Thompson, R. J., Zhou, N., and Mac Vicar, B. A. (2006). Ischemia opens neuronal gap junction hemichannels. Science 312, 924-27). At least 10 min was allowed for neurons to adapt to the change from culture medium to aCSF before OGD was applied.

### Real-Time Monitoring of Mitochondrial Function

Mitochondria function was evaluated using Rhodamine-123 dequenching as previously reported. Rhodamine-123 (Rh123, Thermal Fisher Scientific, R302) was dissolved in DMSO to make a stock concentration at 10 mg/mL. Pre-warmed Neurobasal® Medium was used to dilute Rhodamine-123 to 5 μg/mL as working concentration. Culture medium was removed and cultured neurons on the 25 mm coverslip were washed using prewarmed PBS for 3 times, then 2 ml of Rh123 working solution was added. Cells were incubated with Rh123 at 37° C. for 5 min. Then Rh123 working solution was replaced with culture medium. At least 10 min were allowed to achieve Rh123 equilibration after the transition of culture medium to aCSF before experiments.

Fluorescence intensities at 509 nm with excitation at 488 nm was collected every 15 s for 30 min using CoolSNAP HQ2 (Photometrics) and data were analyzed using NIS-Elements (Nikon).

### Ratio Calcium Imaging Experiments

Changes of intracellular Ca2+ was measured using ratio Ca2+ imaging as we describe previously (Du, J., Xie, J., Zhang, Z., Tsujikawa, H., Fusco, D., Silverman, D., Liang, B., and Yue, L. (2010). TRPM7-mediated Ca2+ signals confer fibrogenesis in human atrial fibrillation. Circulation research 106, 992-1003). In brief, Fura-2 AM (Thermal Fisher Scientific, F1221) was dissolved in DMSO to make a stock concentration at 1 mM. Pre-warmed Neurobasal® Medium (Thermal Fisher Scientific, 21103-049) was used to dilute Fura-2 AM to a working concentration at 2.5 μM, and 0.02% Pluronic™ F-127 (Thermal Fisher Scientific, P3000MP) was added to facilitate loading of Fura-2 AM. Cells plated on 25 mm glass coverslips were washed using pre-warmed PBS for 3 times, and then incubated with 2 ml of Fura-2 AM working solution for 30˜45 min at 37° C. Non-incorporated dye was washed away using HEPES-buffered Saline Solution (HBSS) containing (in mM): 20 HEPES, 10 glucose, 1.2 MgCl2, 1.2 KH2PO4, 4.7 KCl, 140 NaCl, 1.3 Ca2+ (pH 7.4).

Ca2+ influx was measured by perfusing the cells with Tyrode's solution for transfected HEK293T cells or aCSF for neurons under different conditions. Ionomycin (Iono) at 1 μM was applied at the end of the experiment as an internal control. Fluorescence intensities at 510 nm with 340 nm and 380 nm excitation were collected at a rate of 1 Hz using CoolSNAP HQ2 (Photometrics) and data were analyzed using NIS-Elements (Nikon). The 340:380 nm ratio in the presence of different treatments was normalized to that of maximal Ca2+ signal elicited by 1 μM Ionomycin (Iono) as we previously reported (Du, J., Xie, J., Zhang, Z., Tsujikawa, H., Fusco, D., Silverman, D., Liang, B., and Yue, L. (2010). TRPM7-mediated Ca2+ signals confer fibrogenesis in human atrial fibrillation. Circulation research 106, 992-1003).

### Co-Immunoprecipitation

NP-40 lysis buffer (10% NP40, 150 mM NaCl, 1 mM EDTA, 50 mM Tris, pH=8.0) containing proteinase inhibitors (Sigma-Aldrich, 539131-10VL) and phosphatase inhibitors (Thermal Fisher Scientific, 78428) was used to lyse both cultured cells and frozen brain tissue. For transfected cells, proteins were extracted 36 hours after transfection. Cell and tissue lysate were lysed by ultrasound using an ultrasonic cleaner (Thermal Fisher Scientific) filled with ice-cold water for 30 min. After incubated on ice for 1 h, lysate was centrifuged at 13000 g for 30 min and supernatant was collected. Protein concentration was measured using Pierce™ Rapid Gold BCA Protein Assay Kit (Thermal Fisher Scientific, A53225). 300 μg of protein was taken and diluted using NP-40 lysis buffer to make a total volume of 500 μl. Unused protein was allocated and frozen at −80° C. for future use. Appropriate amount of antibody was added based on instruction. After protein-antibody mixture was incubated on ice for 2 h, 25 μL of pre-washed Protein A/G PLUS-Agarose (Santa Cruz Biotechnology, sc-2003) was added, and the whole mixture was incubated at 4° C. for overnight. Then the mixture was centrifuged at 2500 g for Imin to get agarose beads. Agarose beads was washed using NP-40 lysis buffer for 7 times, mixed with same amount of 2× Laemmli Sample Buffer (BIO-RAD, 1610737), and boiled at 95° C. for 5 min. Then samples were ready for western blotting analysis.

### Western Blotting

NP-40/Triton lysis buffer (10% NP40, 1% Triton™ X-100, 150 mM NaCl, 1 mM EDTA, 50 mM Tris, pH=8.0) containing proteinase inhibitors and phosphatase inhibitors was used to lyse both cultured cells and frozen brain tissue. Surface protein was extracted using Pierce® Cell Surface Protein Isolation Kit (Thermal Fisher Scientific, 89881) in transfected HEK-293T cells, and using ProteoExtract™ Native Membrane Protein Extraction Kit (Calbiochem, 444810) in brain tissue based on instructions. For transfected cells, proteins were extracted 36 hours after transfection. Cell and tissue lysate were lysed by ultrasound using an ultrasonic cleaner filled with ice-cold water for 30 min. After incubated on ice for 1 h, lysate was centrifuged at 13000 g for 30 min and supernatant was collected. Protein concentration was measured using Pierce™ Rapid Gold BCA Protein Assay Kit.

30-50 μg of total protein was loaded and separated proteins were transferred to Nitrocellulose membranes. Membranes were blocked with 5% BSA and 2.5% goat serum in Tris buffered saline (TBS, pH=7.4) at room temperature for 2 h, and incubated with primary antibodies in TBS with 0.05% Tween (TBS-T) at room temperature for 2 h. Then membranes were incubated with secondary antibodies in TBS-T for 1 h at room temperature for 1 h for detection. Blots were developed with ImageQuant LAS 4000 imaging system. Band intensity was quantified using ImageJ software and normalized with appropriate loading controls.

### Electrophysiology

Whole cell currents were recorded using an Axopatch 200B amplifier. Data were digitized at 10 or 20 kHz and digitally filtered offline at 1 kHz. Patch electrodes were pulled from borosilicate glass and fire-polished to a resistance of ˜3 MΩ when filled with internal solutions. Series resistance (Rs) was compensated up to 90% to reduce series resistance errors to <5 mV. Cells in which Rs was >10 MΩ were discarded (Du, J., Xie, J., and Yue, L. (2009). Modulation of TRPM2 by acidic pH and the underlying mechanisms for pH sensitivity. J Gen Physiol 134, 471-88).

For heterologous expression, transfected HEK-293 cells were identified by GFP fluorescence. TRPM2 current recording in transfected HEK-293T cells was performed as we previously reported (Du, J., Xie, J., and Yue, L. (2009). Intracellular calcium activates TRPM2 and its alternative spliced isoforms. Proceedings of the National Academy of Sciences 107, 7239-44; Du, J., Xie, J., and Yue, L. (2009). Modulation of TRPM2 by acidic pH and the underlying mechanisms for pH sensitivity. J Gen Physiol 134, 471-88). TRPM2 and NMDAR currents recordings from cultured neurons were performed using sCSF as extracellular solution as we previously reported (Zeng, H., Guo, M., Martins-Taylor, K., Wang, X., Zhang, Z., Park, J. W., Zhan, S., Kronenberg, M. S., Lichtler, A., Liu, H. X., et al. (2010). Specification of region-specific neurons including forebrain glutamatergic neurons from human induced pluripotent stem cells. PloS one 5, e11853). In brief, for TRPM2 current recordings, voltage stimuli lasting 250 ms were delivered at 1-s intervals, with voltage ramps ranging from −100 to +100 mV at holding potential of 0 mV to elicited currents. For NMDA current recordings, a gap-free protocol at holding potential of −80 mV was applied to elicit NMDA currents upon agonist stimulation. A fast perfusion system was used to exchange extracellular solutions and to deliver agonists and antagonists to the cells, with a complete solution exchange achieved in about 1-3 s (Jiang, J., Li, M., and Yue, L. (2005). Potentiation of TRPM7 inward currents by protons. J Gen Physiol 126, 137-50).

Normal Tyrode solution contained (mM): 145 NaCl, 5 KCl, 2 CaCl2, 10 HEPES, 10 glucose, osmolarity=290-320 mOsm/Kg, and pH=7.4 was adjusted with NaOH. Extracellular solution for current recording in neuron, the aCSF, solution contained (mM): 124 NaCl, 2.5 KCl, 2 MgSO4, 2 CaCl2, 1.2 NaH2PO4, 24 NaHCO₃, 5 HEPES, 12.5 glucose, osmolarity=300-310 mOsm/Kg, with pH=7.4 adjusted with NaOH. For oxygen-glucose-deprivation (OGD) solution, glucose was eliminate from extracellular solution, and the solution was saturated with nitrogen (N2) bubbling for 30 min before the experiments.

The internal pipette solution for whole cell current recordings of TRPM2 contained (in mM): 135 Cs-methanesulfonate (CsSO3CH3), 8 NaCl, 0.5 CaCl2, 1 EGTA, and 10 HEPES, with pH adjusted to 7.2 with CsOH. Free [Ca2+]i buffered by EGTA was 100 nM calculated using Max chelator (Du, J., Xie, J., and Yue, L. (2009). Modulation of TRPM2 by acidic pH and the underlying mechanisms for pH sensitivity. J Gen Physiol 134, 471-88). ADPR 200 UM was included in the pipette solution for most experiments. The intracellular pipette solution to test the effects of OGD on TRPM2 currents in neuron was adjusted to sub-optimal condition, containing (in mM) 135 CsCH3SO4, 8 NaCl, 0.01 CaCl2, 1 MgCl2, 10 HEPES (pH 7.2) and 10 μM ADPR.

The intracellular solution for NMDAR current recording contained (mM): 110 K-ASP, 20 KCl, 1 MgSO4, 0.05 mM EGTA-K+, 0.1 GTP, 5 ATP-Mg2, 10 HEPES, osmolarity=275-285 mOsm/Kg, pH=7.2 adjusted with KOH. For the experiments using cells pretreated with the disrupting peptides TAT-SC and TAT-EE3, 10 μM TAT-SC or TAT-EE3 was included in the pipette solution, and at least 10 min was allowed for achieving intracellular equilibration of TAT-SC or TAT-EE3 before current recording.

For current recordings in neurons, tetrodotoxin (0.5 μM) was included in the external solution to block voltage-gated Na+ current, and 10 μM nifedipine was used to block voltage-gated Ca2+ currents for recording TRPM2 currents.

### Immunofluorescence Staining

Brains harvested from mice were frozen at −80° C. prior to use, and was mounted in Fisher Healthcare™ Tissue-Plus™ O.C.T. Compound (Thermal Fisher Scientific, 23-730-571) prior to cutting. Brains were cut into sagittal slices at a thickness of 6 μM to 8 μM, mounted to Superfrost® Plus Microscope Slides (Thermal Fisher Scientific, 12-550-15), and frozen at −80° C. for future use. Prior to staining, slides were taken to room temperature for at least 30 min allowing for dehydration. Slices were fixed in 10% formaldehyde for 15 min following washing using PBS for 3 times, and incubated in blocking solution containing 5% BSA, 15% goat serum and 1% Triton X-100 at room temperature for 2 h. Primary antibodies were diluted as described previously in TBS-T containing 15% goat serum. Slices were incubated with primary antibodies for at least 12 h at 4° C. following washing using PBS for 3 times, and incubated with secondary antibodies at room temperature for 2 h. Then slices were washed using PBS for 3 times, and mounted using Prolong® Gold antifade reagent with DAPI. Slices were kept at 4° C. before taking pictures. TUNEL staining was performed based on the instruction of kit.

### Data Analysis

All data are expressed as mean±SEM. For two groups' comparison, statistical significance was determined using Student's t-test. For multiple groups' comparison, statistical significance was determined using one-way or two-way analysis of variance (ANOVA) flowed by Bonferroni posttest. P<0.05 was regarded as significant.

TABLE 2
Primers for subcloning, mutagenesis and genotyping

| Application | Genes | Primers | Primer sequences (5′-3′) |
|---|---|---|---|
| Subcloning | Trpm2 C | F | CTCGAATTCTGAAGGAGAACTACCTCCA |
| | terminal part | | GAAC (SEQ ID NO: 12) |
| | | R | GATCTAGATTAGGTCTTGTGGTTCGCATA |
| | | | GAGTG (SEQ ID NO: 13) |
| | GluN2a C | F | CGGAATTCCGACACTCTTCTACTGGAAG |
| | terminal part | | (SEQ ID NO: 14) |
| | | R | TGCTCTAGAGCTTAAACATCAGATTCGA |
| | | | TACTAGG (SEQ ID NO: 15) |
| | GluN2b C | F | CGGAATTCCGTCATCACCTTCATCTGTGA |
| | terminal part | | G (SEQ ID NO: 16) |
| | | R | TGCTCTAGAGCACCTTAACCTCTCTCTCT |
| | | | TC (SEQ ID NO: 17) |
| Mutagenesis | Trpm2 1-727 | F | CAAGGACATGTAGTTTGTGTC (SEQ ID |
| | | | NO: 18) |
| | | R | GACACAAACTACATCATGTCCTTG (SEQ |
| | | | ID NO: 19) |
| | Trpm2 1-679 | F | TGGCGCTGGCGTAGTAGTATG (SEQ ID |
| | | | NO: 20) |
| | | R | TCATACTACTACGCCAGCGCC (SEQ ID |
| | | | NO: 21) |
| | Trpm2 1-631 | F | ATTTGGGCCATTGTCTAGAACCGT (SEQ |
| | | | ID NO: 22) |
| | | R | ACGGTTCTAGACAATGGCCCAAATG |
| | | | (SEQ ID NO: 23) |
| | Trpm2 1-570 | F | TGCTGGGGGAATTCACGCAG (SEQ ID |
| | | | NO: 24) |
| | | R | TGCGTGAATTCCCCCAGCAG (SEQ ID NO: |
| | | | 25) |
| | Trpm2 1-664 | F | TGAAGGAACTGTCCTAGGAGGAGGAG |
| | | | (SEQ ID NO: 26) |
| | | R | TCCTCCTCCTAGGACAGTTCCTTCAG |
| | | | (SEQ ID NO: 27) |
| | Trpm2 EE3 | F | AAGATCCTGAAGGAACTGTCCAAGTATG |
| | deletion | | AGCACAGAGCCATC (SEQ ID NO: 28) |
| | | R | GATGGCTCTGTGCTCATACTTGGACAGTT |
| | | | CCTTCAGGATCTT (SEQ ID NO: 29) |
| | Trpm2 QEE | F | AAGGAACTGTCCAAGCAGCAGGAGGAC |
| | | | ACGGAC (SEQ ID NO: 30) |
| | | R | TCCGTGTCCTCCTGCTGCTTGGACAGTTC |
| | | | CTTC (SEQ ID NO: 31) |
| | Trpm2 EQE | F | ACACGGACAGCTCGCAGCAGATGCTGGC |
| | | | G (SEQ ID NO: 32) |
| | | R | CGCCAGCATCTGCTGCGAGCTGTCCGTG |
| | | | TC (SEQ ID NO: 33) |
| | Trpm2 EEQ | F | TGGCGCTGGCGCAGCAGTATGAGCACAG |
| | | | AG (SEQ ID NO: 34) |
| | | R | TCTGTGCTCATACTGCTGCGCCAGCGCC |
| | | | AG (SEQ ID NO: 35) |
| | GluN2a 1- | F | ACCTTCATCTGGTAGCACCTCTTCTAC |
| | 1053 | | (SEQ ID NO: 36) |
| | | R | TAGAAGAGGTGCTACCAGATGAAGGTG |
| | | | (SEQ ID NO: 37) |
| | GluN2a 1- | F | ACCTTCATCTGTTAGCATCTGTTCTATTG |
| | 1047 | | (SEQ ID NO: 38) |
| | | R | AATAGAACAGATGCTAACAGATGAAGGT |
| | | | G (SEQ ID NO: 39) |
| | GluN2a KKR | F | TATGATAACATTCTGGACAAACCCAG |
| | deletion | | (SEQ ID NO: 40) |
| | | R | GAACTGGAGGGCGTTGTT |
| | | | (SEQ ID NO: 41) |
| | GluN2b KKR | F | TCCTACGACACCTTCGTG (SEQ ID NO: 42) |
| | deletion | | |
| | | R | CTGAGCCTTGGAATTAGTCGG (SEQ ID NO: 43) |
| E. coli | | | |
| expression | | | |
| | Trpm2 EE3 | F | ATATGCGGCCGCTCAGGCCATGTGACCT |
| | | | TCAC (SEQ ID NO: 44) |
| | | | ATATGGTACCTTACTTCATGTCCTTGGCC |
| | | R | TCCAG (SEQ ID NO: 45) |
| | | | ATATGCGGCCGCTCAGGCCATGTGACCT |
| | Trpm2 EQE | F | TCAC (SEQ ID NO: 44) |
| | | | ATATGGTACCTTACTTCATGTCCTTGGCC |
| | | R | TCCAG (SEQ ID NO: 45) |
| | | | ATATGCGGCCGCTCTCCTTTCAAGTGTGA |
| | GluN2a KKR | F | TGC (SEQ ID NO: 46) |
| | | | ATATGGTACCTTAGCT |
| | | R | TTTGTTCCCCAAGAGTTT (SEQ ID NO: 47) |
| | | | ATATGCGGCCGCGAGGCCTGTAAGAAGG |
| | Glu2b KK4 | F | CT (SEQ ID NO: 49) |
| | | R | ATATGGTACCTTATGAGGACTTGTTGGC |
| | | | AAA G (SEQ ID NO: 49) |
| Genotyping | Cre | F | GATATCTCACGTACTGACGG (SEQ ID NO: |
| | | | 50) |
| | | R | TGACCAGAGTCATGGTTAGC (SEQ ID NO: |
| | | | 51) |
| | Trpm2 loxp | F | GGCTCTGCCTCATCCCCAGAATC (SEQ ID |
| | | | NO: 52) |
| | | R | CCGGATACAGATGCAGGATGCTG (SEQ |
| | | | ID NO: 53) |
| | | R | CTGAAGGTCCTGAGTTTGAATCCCA (SEQ |
| | | | ID NO: 54) |
| | TRPM2-KO | F | CTTGGGTTGCAGTCATATGCAGGC (SEQ |
| | | | ID NO: 55) |
| | | R | GCCCTCACCATCCGCTTCACGATG (SEQ |
| | | | ID NO: 56) |
| | | R | GCCACACGCGTCACCTTAATATGC (SEQ |
| | | | ID NO: 112) |

1. A method for treating or preventing neurological injury or neurological disorder in a subject comprising administering to the subject an agent that inhibits the interaction between N-methyl-D-aspartate receptor (NMDAR) and transient receptor potential cation channel subfamily M member 2 (TRPM2).

2. The method of claim 1, (a) wherein the agent targets a NMDAR-binding site on TRPM2; or (b) wherein the agent targets a TRPM2-binding site on NMDAR.

3. The method of claim 2,
(a) wherein the NMDAR-binding site comprises residues 631 to 679 or residues 665-681 of TRPM2; or an amino acid sequence of EEEDTDSSEEMLALAEE (SEQ ID NO:3); and/or
(b) wherein the TRPM2-binding site comprises an amino acid sequence of QFQKNKLRINRQHSYD (SEQ ID NO:5), an amino acid sequence of QKNKLRINRQHS (SEQ ID NO:6), an amino acid sequence of AQKKNRNKLRRQHSY (SEQ ID NO:7), or an amino acid sequence of KKNRNKLRROH (SEQ ID NO:8).

4. (canceled)

5. The method of claim 1 any one of claims 1-4,
(a) wherein the agent comprises a peptide comprising an amino acid sequence with at least 80%, 85%, 90% or 95% sequence identity to the amino acid sequence of EEEDTDSSEEMLALAEE (SEQ ID NO:3) or the amino acid sequence of YGRKKRRORRREEDTDSSEEMLALAEE (SEQ ID NO:4);
(b) wherein the peptide comprises an amino acid sequence differing by 1, 2, 3, 4, or 5 residues from the amino acid sequence of EEEDTDSSEEMLALAEE (SEQ ID NO:3) or the amino acid sequence of YGRKKRRQRRREEDTDSSEEMLALAEE (SEQ ID NO:4);
(c) wherein the peptide comprises the amino acid sequence of
(SEQ ID NO: 4)
YGRKKRRQRRREEDTDSSEEMLALAEE;
(d) wherein the peptide does not comprise the amino acid sequence of
(SEQ ID NO: 3)
DTDSSEEMLALAEE;
(e) wherein the agent comprises a small molecule that binds to the NMDAR-binding site;
(f) wherein the agent comprises an antagonist anti-TRPM2 antibody that binds to the NMDAR-binding site;
(g) wherein the agent comprises a mutant TRPM2 protein, wherein the mutant TRPM2 protein comprises a deletion of the NMDAR-binding site; and/or
(h) wherein the mutant TRPM2 protein comprises a substitution mutation at residue 674, a substitution mutation at residue 675, or substitution mutations at both residues 674 and 675 of TRPM2 protein.

6-15. (canceled)

16. The method of claim 1,
(a) wherein the agent comprises a peptide comprising an amino acid sequence with at least 80%, 85%, 90% or 95% sequence identity to the amino acid sequence of QFQKNKLRINRQHSYD (SEQ ID NO:5), or the amino acid sequence of QKNKLRINRQHS (SEQ ID NO:6);
(b) wherein the peptide comprises an amino acid sequence differing by 1, 2, 3, 4, or 5 residues from the amino acid sequence of QFQKNKLRINRQHSYD (SEQ ID NO:5), or the amino acid sequence of QKNKLRINRQHS (SEQ ID NO:6);
(c) wherein the peptide does not comprise the amino acid sequence of QFQKNKLRINRQHSYD (SEQ ID NO:5), or the amino acid sequence of QKNKLRINRQHS (SEQ ID NO:6);
(d) wherein the agent comprises a peptide comprising an amino acid sequence with at least 80%, 85%, 90% or 95% sequence identity to the amino acid sequence of AQKKNRNKLRRQHSY (SEQ ID NO:7), or the amino acid sequence of KKNRNKLRROH (SEQ ID NO:8);
(e) wherein the peptide comprises an amino acid sequence differing by 1, 2, 3, 4, or 5 residues from the amino acid sequence of AQKKNRNKLRRQHSY (SEQ ID NO:7), or the amino acid sequence of KKNRNKLRROH (SEQ ID NO:8);
(f) wherein the peptide does not comprise the amino acid sequence of AQKKNRNKLRRQHSY (SEQ ID NO:7), or the amino acid sequence of KKNRNKLRRQH (SEQ ID NO:8);
(g) wherein the agent comprises a small molecule that binds to the TRPM2-binding site;
(h) wherein the agent comprises an antagonist anti-NMDAR antibody that binds to the TRPM2-binding site; and/or
(i) wherein the agent comprises a mutant NMDAR protein, wherein the mutant NMDAR protein comprises a deletion of the TRPM2-binding site.

17-24. (canceled)

25. The method of claim 1,
(a) wherein the neurological injury results from a brain injury;
(b) wherein the brain injury is selected from the group consisting of stroke, traumatic brain injury, cerebral palsy, acquired brain injury, anoxic brain injury, diffuse axonal brain injury, focal brain injury, subdural hematoma, brain aneurysm, and coma;
(c) wherein the brain injury is ischemic stroke, hemorrhagic stroke, or transient ischemic attack;
(d) wherein the neurological disorder is a neurodegenerative disease;
(e) wherein the neurodegenerative disease is selected from the group consisting of Alzheimer's Disease, multiple sclerosis, HIV-associated dementia, Huntington's Disease, Parkinson's Disease, and amyotrophic lateral sclerosis; and/or
(f) wherein the subject is a human subject.

26-29. (canceled)

30. The method of claim 1
(a) wherein administering to the subject the agent inhibits the non-excitotoxic calcium signaling pathway mediated by TRPM2;
(b) wherein administering to the subject the agent inhibits the excitotoxic calcium signaling pathway mediated by NMDAR;
(c) wherein administering to the subject the agent decreases the NMDAR surface expression in neurons;
(d) wherein administering to the subject the agent decreases the NMDAR current in neurons;
(e) wherein administering to the subject the agent reduces mitochondrial membrane depolarization induced by ischemic injury in neurons;
(f) wherein administering to the subject the agent prevents neuronal death induced by ischemic injury; and/or
(g) wherein administering to the subject the agent reduces infarct volume and/or improves neurological behavior score.

31. A peptide comprising an amino acid sequence with at least 80% or 85% sequence identity to
(a) the amino acid sequence of YGRKKRRQRRREEDTDSSEEMLALAEE (SEQ ID NO: 4), or
(b) the amino acid sequence of EEEDTDSSEEMLALAEE (SEQ ID NO:3), or
(c) the amino acid sequence of QFQKNKLRINRQHSYD (SEQ ID NO:5), or
(d) the amino acid sequence of QKNKLRINRQHS (SEQ ID NO:6), or
(e) the amino acid sequence of AQKKNRNKLRRQHSY (SEQ ID NO:7), or
(f) the amino acid sequence of KKNRNKLRROH (SEQ ID NO:8), or a multimer, derivative, or variant thereof.

32. (canceled)

33. (canceled)

34. The peptide of claim 31,
(a) wherein the peptide comprises an amino acid sequence differing by 1, 2, 3, 4, or 5 residues from the amino acid sequence of YGRKKRRQRRREEDTDSSEEMLALAEE (SEQ ID NO: 4); the amino acid sequence of EEEDTDSSEEMLALAEE (SEQ ID NO:3); the amino acid sequence of QFQKNKLRINRQHSYD (SEQ ID NO:5), the amino acid sequence of QKNKLRINRQHS (SEQ ID NO:6), the amino acid sequence of AQKKNRNKLRRQHSY (SEQ ID NO:7); or the amino acid sequence of KKNRNKLRROH (SEQ ID NO:8),
(b) wherein the peptide does not comprise the amino acid sequence of EEEDTDSSEEMLALAEE (SEQ ID NO:3), the amino acid sequence of QFQKNKLRINRQHSYD (SEQ ID NO:5), the amino acid sequence of QKNKLRINRQHS (SEQ ID NO:6), the amino acid sequence of AQKKNRNKLRRQHSY (SEQ ID NO:7); or the amino acid sequence of KKNRNKLRRQH (SEQ ID NO:8), and/or
(c) wherein the peptide comprises the amino acid sequence of
(SEQ ID NO: 4)
YGRKKRRQRRREEDTDSSEEMLALAEE.

35-50. (canceled)

51. The peptide of claim 31,
(a) wherein the peptide is isolated from a cell;
(b) wherein the peptide is a synthetic peptide;
(c) wherein the peptide inhibits interaction between NMDAR to TRPM2;
(d) wherein the peptide inhibits the non-excitotoxic calcium signaling pathway mediated by TRPM2;
(e) wherein the peptide inhibits the excitotoxic calcium signaling pathway mediated by NMDAR;
(f) wherein the peptide decreases the NMDAR surface expression in neurons;
(g) wherein the peptide decreases the NMDAR current in neurons;
(h) wherein the peptide reduces mitochondrial membrane depolarization induced by ischemic injury in neurons;
(i) wherein the peptide prevents neuronal death induced by ischemic injury; and/or
(j) wherein the peptide is a cell permeable peptide.

52-60. (canceled)

61. A nucleic acid molecule encoding the peptide of claim 31.

62. An expression vector comprising the nucleic acid molecule of claim 61 operably linked to a control sequence for the expression of the peptide.

63. (canceled)

64. A pharmaceutical composition comprising the peptide of claim 31, and at least one pharmaceutically acceptable excipient.

65. A method for treating or preventing neurological injury or neurological disorder in a subject comprising administering to the subject the peptide of claim 31.

66. The method of claim 65,
(a) wherein administering to the subject the peptide inhibits the interaction between TRPM2 and NMDAR;
(b) wherein administering to the subject the peptide inhibits the non-excitotoxic calcium signaling pathway mediated by TRPM2;
(c) wherein administering to the subject the peptide inhibits the excitotoxic calcium signaling pathway mediated by NMDAR;
(d) wherein administering to the subject the peptide decreases the NMDAR surface expression in neurons;
(e) wherein administering to the subject the peptide decreases the NMDAR current in neurons;
(f) wherein administering to the subject the peptide reduces mitochondrial membrane depolarization induced by ischemic injury in neurons;
(g) wherein administering to the subject the peptide prevents neuronal death induced by ischemic injury; and/or
(h) wherein administering to the subject the peptide reduces infarct volume and/or improves neurological behavior score.

67-73. (canceled)

74. The method of claim 65,
(a) wherein the neurological injury results from a brain injury;
(b) wherein the brain injury is selected from the group consisting of stroke, traumatic brain injury, cerebral palsy, acquired brain injury, anoxic brain injury, diffuse axonal brain injury, focal brain injury, subdural hematoma, brain aneurysm, and coma;
(c) wherein the brain injury is ischemic stroke, hemorrhagic stroke, or transient ischemic attack;
(d) wherein the neurological disorder is a neurodegenerative disease;
(e) wherein the neurodegenerative disease is selected from the group consisting of Alzheimer's Disease, multiple sclerosis, HIV-associated dementia, Huntington's Disease, Parkinson's Disease, and amyotrophic lateral sclerosis; and/or
(f) wherein the subject is a human subject.

75-79. (canceled)

80. The method of claim 1, further comprising administering to the subject an additional therapeutic agent.

81-82. (canceled)

83. A method for identifying a compound useful for treating or preventing neurological injury or neurological disorder in a subject in need thereof, comprising
a. providing a test compound,
b. determining the effect of the test compound on the interaction between TRPM2 and NMDAR, and
c. selecting a compound that inhibits the interaction between TRPM2 and NMDAR, thereby identifying a compound useful for treating or preventing neurological injury or neurological disorder in the subject.

84. The method of claim 83, (a) wherein the compound binds to a NMDAR-binding site on TRPM2; or (b) wherein the compound binds to a TRPM2-binding site on NMDAR.

85. The method of claim 84,
(a) wherein the NMDAR-binding site comprises residues 631 to 679 or residues 665-681 of TRPM2;
(b) wherein the NMDAR-binding site comprises an amino acid sequence of
(SEQ ID NO: 3)
EEEDTDSSEEMLALAEE;
(c) wherein the TRPM2-binding site comprises an amino acid sequence of QFQKNKLRINRQHSYD (SEQ ID NO:5), or an amino acid sequence of QKNKLRINRQHS (SEQ ID NO:6); and/or
(d) wherein the TRPM2-binding site comprises an amino acid sequence of AQKKNRNKLRRQHSY (SEO ID NO: 7), or an amino acid sequence of KKNRNKLRROH (SEQ ID NO:8).

86-89. (canceled)
